(19) United States
(12) Patent Application Publication
(10) Pub. No.: US 2024/0200059 A1
(43) Pub. Date: Jun. 20, 2024

(54) PHOTOCLEAVABLE GUIDE RNAS AND METHODS OF USE THEREOF

(71) Applicant: VOR BIOPHARMA INC., Cambridge, MA (US)

(72) Inventors: Dane Hazelbaker, Cambridge, MA (US); Elizabeth Paik, Cambridge, MA (US)

(73) Assignee: VOR BIOPHARMA INC., Cambridge, MA (US)

(21) Appl. No.: 18/285,765

(22) PCT Filed: Apr. 8, 2022
(86) PCT No.: PCT/US2022/024069

§ 371 Date: Oct. 5, 2023

Related U.S. Application Data
(60) Provisional application No. 63/191,509, filed on May 21, 2021.
(60) Provisional application No. 63/173,212, filed on Apr. 9, 2021.

(51) Int. Cl. C12N 15/11; C12N 9/22; C12N 15/90

(52) U.S. Cl. CPC C12N 15/11; C12N 9/22; C12N 15/907; C12N 2310/20

(57) ABSTRACT

Provided herein are photocleavable guide RNAs (gRNAs) comprising a targeting domain, a CRISPR RNA (crRNA) sequence for a CRISPR/Cas nuclease, and a at least one photocleavable moiety. Also provided herein are methods involving contacting a photocleavable gRNA and a CRISPR/Cas nuclease to form a CRISPR system. Also provided herein are methods involving providing a cell and contacting the photocleavable gRNA and a CRISPR/Cas nuclease, thus forming a ribonucleoprotein complex that binds to a target site sequence in the genome of the cell.

### RELATED APPLICATIONS

This application claims the benefit under 35 U.S.C. 119(e) of U.S. provisional application No. 63/173,212, filed Apr. 9, 2021, and U.S. provisional application No. 63/191,509, filed May 21, 2021, each of which is incorporated by reference herein in its entirety.

### BACKGROUND

Clustered regulatory Interspaced Short Palindromic Repeats (CRISPR)/Cas systems a provide a platform for targeted gene editing in cells. Despite the versatility of the systems and associated tools for use, there are a number of potentially risks associated with genetic modification using CRISPR/Cas systems, such as off-target effects, risk of translocation events, and potential malignancy. These challenges are safety concerns for use of CRISPR/Cas systems in therapeutic application.

### SUMMARY

Aspects of the present disclosure provide a guide RNA (gRNA) comprising a targeting domain; a CRISPR RNA (crRNA) sequence for a CRISPR/Cas nuclease; and at least one photocleavable moiety. In some embodiments, the gRNA comprises 1, 2, 3, 4, 5, or more photocleavable moieties. In some embodiments, the photocleavable moiety is a 2-nitrobenzyl bond. In some embodiments, the targeting domain is corresponding to a target site sequence adjacent to a protospacer-adjacent motif (PAM) in a gene. In some embodiments, the crRNA sequence comprises a first complementarity domain, a linking domain, and a second complementarity domain that is complementary to the first complementarity domain; and a proximal domain.

In some embodiments, the at least one photocleavable moiety is located within the targeting domain. In some embodiments, the at least one photocleavable moiety is located within the targeting domain, wherein the photocleavable moiety is located between two nucleotides of the targeting domain at a position corresponding to a nucleotide of the target site sequence. In some embodiments, the at least one photocleavable moiety is located following nucleotide position 1, 2, 3, 4, 5, 6, 7, 8, 9, 10, 11, 12, 13, 14, 15, 16, 17, 18, 19, 20, 21, or 22 from the 5′ end of the targeting domain. In some embodiments, the at least one photocleavable moiety is located following nucleotide position 1, 2, 3, 4, 5, 6, 7, 8, 9, 10, 11, 12, 13, 14, 15, 16, 17, 18, 19, 20, 21, or 22 from the 5′ end of the gRNA. In some embodiments, the at least one photocleavable moiety is located at any one or more of positions 9, 11, 13, 15, or 17 from the 5′ end of the targeting domain. In some embodiments, the at least one photocleavable moiety is located at any one or more of positions 9, 11, 13, 15, or 17 from the 5′ end of the gRNA. In some embodiments, the at least one photocleavable moiety is located between nucleotide positions 1 and 3, 2 and 4, 3 and 5, 4 and 6, 5 and 7, 6 and 8, 7 and 9, 8 and 10, 9 and 11, 10 and 12, 11 and 13, 12 and 14, 13 and 15, 14 and 16, 15 and 17, 16 and 18, 17 and 19, 18 and 20, 19 and 21, or 20 and 22 from the 5′ end of the targeting domain. In some embodiments, the at least one photocleavable moiety is located between nucleotide positions 1 and 3, 2 and 4, 3 and 5, 4 and 6, 5 and 7, 6 and 8, 7 and 9, 8 and 10, 9 and 11, 10 and 12, 11 and 13, 12 and 14, 13 and 15, 14 and 16, 15 and 17, 16 and 18, 17 and 19, 18 and 20, 19 and 21, or 20 and 22 from the 5′ end of the gRNA. In some embodiments, the at least one photocleavable moiety is located within 5, 10, or 15 nucleotides from the 3′ end of the targeting domain.

In some embodiments, the at least one photocleavable moiety is located within the targeting domain following a position corresponding to the nucleotide at a position 1, 2, 3, 4, 5, 6, 7, 8, 9, 10, 11, 12, 13, 14, 15, 16, 17, 18, 19, 20, 21, or 22 nucleotides adjacent to the PAM. In some embodiments, the at least one photocleavable moiety is located within the targeting domain between nucleotide positions 1 and 3, 2 and 4, 3 and 5, 4 and 6, 5 and 7, 6 and 8, 7 and 9, 8 and 10, 9 and 11, 10 and 12, 11 and 13, 12 and 14, 13 and 15, 14 and 16, 15 and 17, 16 and 18, 17 and 19, 18 and 20, 19 and 21, 20 and 22, or 21 and 23 adjacent to the PAM. In some embodiments, the at least one photocleavable moiety is located within the targeting domain following a position corresponding to the nucleotide at one or more of positions 9, 11, 13, 15, or 17 nucleotides adjacent to the PAM.

In some embodiments, the at least one photocleavable moiety is located within the crRNA sequence. In some embodiments, the at least one photocleavable moiety is located within the crRNA sequence, wherein the photocleavable moiety is located between two nucleotides of the crRNA replacing a nucleotide of a corresponding wildtype crRNA sequence.

In some embodiments, the at least one photocleavable moiety is located following nucleotide position 1, 2, 3, 4, 5, 6, 7, 8, 9, 10, 11, 12, 13, 14, 15, 16, 17, 18, 19, or 20 from the 5′ end of the crRNA sequence. In some embodiments, the at least one photocleavable moiety is located following nucleotide position 1, 2, 3, 4, 5, 6, 7, 8, 9, 10, 11, 12, 13, 14, 15, 16, 17, 18, 19, or 20 from the 5′ end of the gRNA. In some embodiments, the at least one photocleavable moiety is located within 5, 10, or 15 nucleotides from the 3′ end of the crRNA sequence. In some embodiments, the at least one photocleavable moiety is located within 5, 10, or 15 nucleotides from the 3′ end of the gRNA. In some embodiments, the at least one photocleavable moiety is located between nucleotide positions 1 and 3, 2 and 4, 3 and 5, 4 and 6, 5 and 7, 6 and 8, 7 and 9, 8 and 10, 9 and 11, 10 and 12, 11 and 13, 12 and 14, 13 and 15, 14 and 16, 15 and 17, 16 and 18, 17 and 19, 18 and 20, 19 and 21 from the 5′ end of the crRNA sequence. In some embodiments, the at least one photocleavable I moiety is located between nucleotide positions 1 and 3, 2 and 4, 3 and 5, 4 and 6, 5 and 7, 6 and 8, 7 and 9, 8 and 10, 9 and 11, 10 and 12, 11 and 13, 12 and 14, 13 and 15, 14 and 16, 15 and 17, 16 and 18, 17 and 19, 18 and 20, or 19 and 21 from the 5′ end of the gRNA.

In some embodiments, the CRISPR/Cas nuclease is a Cas12a (Cpf1) nuclease. In some embodiments, the Cas12a nuclease is AsCas12a, LbCas12a, FnCas12a, or PaCas12a nuclease. In some embodiments, the CRISPR/Cas nuclease is a Cas9 nuclease. In some embodiments, the gRNA is a single guide RNA (sgRNA).

In some embodiments, at least one photocleavable moiety is located within 10, 15, or 20 nucleotides of the 5′ end of the sgRNA. In some embodiments, the gRNA comprises one or more nucleotide residues that are chemically modified. In some embodiments, the gRNA comprises one or more nucleotide residues that comprise a 2′O-methyl moiety, the gRNA comprises one or more nucleotide residues that comprise a phosphorothioate. In some embodiments, the gRNA comprises one or more nucleotide residues that comprise a thioPACE moiety.

In some embodiments, the gRNA is capable of directing the CRISPR nuclease to a target site sequence. In some embodiments, the gRNA comprises a sequence of any one of SEQ ID NOs: 1-40. In some embodiments, the wherein the gRNA comprises a sequence of any one of SEQ ID NOs: 41-43. In some embodiments, the gRNA comprises a sequence of any one of SEQ ID NOs: 134-172, 174, 176, 178, and 180. In some embodiments, the wherein the gRNA comprises a sequence of any one of SEQ ID NOs: 181-190.

Aspects of the present disclosure provide a CRISPR system comprising a CRISPR/Cas nuclease and any of the gRNAs described herein. In some embodiments, the CRISPR/Cas nuclease is a Cas12a (Cpf1) nuclease. In some embodiments, the Cas 12a nuclease is AsCas12a, LbCas12a, FnCas12a, or PaCas 12a nuclease. In some embodiments, the CRISPR/Cas nuclease is a Cas9 nuclease.

Aspects of the present disclosure provide methods comprising contacting any of the gRNAs described herein and a CRISPR/Cas nuclease to form a CRISPR system. In some embodiments, the CRISPR/Cas nuclease is a Cas12a (Cpf1) nuclease. In some embodiments, the Cas12a nuclease is AsCas12a, LbCas12a, FnCas12a, or PaCas12a nuclease. In some embodiments, the CRISPR/Cas nuclease is a Cas9 nuclease.

Aspects of the present disclosure provide methods comprising providing a cell and contacting a cell with (i) any of the gRNAs described herein and (ii) a CRISPR/Cas nuclease, thus forming a ribonucleoprotein (RNP) complex under conditions suitable for the gRNA of (i) to form and/or maintain an RNP complex with the CRISPR/Cas nuclease of (ii), thus forming a CRISPR system, and for the RNP complex to bind a target site sequence in the genome of the cell.

In some embodiments, the contacting comprises introducing (i) and (ii) into the cell in the form of a pre-formed ribonucleoprotein (RNP) complex. In some embodiments, the contacting comprises introducing (i) and/or (ii) into the cell in the form of a nucleic acid encoding the gRNA of (i) and/or the CRISPR/Cas nuclease of (ii). In some embodiments, the nucleic acid encoding the gRNA of (i) and/or the CRISPR nuclease of (ii) is an RNA, preferably an mRNA or an mRNA analog. In some embodiments, the pre-formed RNP complex is introduced into the cell via electroporation.

In some embodiments, the method further comprises contacting the cell with light such that at least 50% of the photocleavable moiety is cleaved. In some embodiments, the method further comprises contacting the cell with light resulting in at least a 50% reduction of cleavage by the CRISPR/Cas nuclease at the target site sequence. In some embodiments, the light has a wavelength of about 350-375 nm. In some embodiments, the light has a wavelength of about 365 nm. In some embodiments, the cell is contacted with light for between 10 seconds and 60 seconds. In some embodiments, the cell is contacted with light for about 30 seconds.

In some embodiments, the cell is a hematopoietic cell. In some embodiments, the cell is a hematopoietic stem cell. In some embodiments, the cell is a hematopoietic progenitor cell. In some embodiments, the cell is an immune effector cell. In some embodiments, the cell is a lymphocyte. In some embodiments, the cell is a T-lymphocyte.

Aspects of the present disclosure provide genetically engineered cells obtained or obtainable by any of the methods described herein. Also provided herein are cell populations comprising any of the genetically engineered cells described herein.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Examples, and the Claims.

### DETAILED DESCRIPTION

Use of CRISPR/Cas systems to effect genetic modifications presents a versatile and adaptable platform, however, there are a number of potential risks associated with CRISPR/Cas use in therapeutic applications, such as off-target effects, risk of translocation events, and potential malignancy. To minimize or reduce potential adverse effects, mechanisms of regulating the activity of the CRISPR/Cas system in the cell, for example to quickly and efficiently induce or terminate its activity and thus DNA cleavage, are desired. Despite existing methods of controlling CRISPR/Cas activity in a cell including anti-CRISPR proteins adapted form bacteriophage, small molecule inhibitors, and oligonucleotide inhibitors, each of these control systems present challenges such as delivery of the inhibitor, incomplete inactivation of the CRISPR/Cas system, and slow kinetics. See, e.g., Harrington et al. 2017; Pawluk et al. 2016; Pawluk et al. 2018; Shin et al. 2017; Kundert et al.; 2019; Maji et al. 2019; Barkau et al. 2019; Li et al. 2018). Zou et al. describe the design of photocleavable gRNAs for use with the Cas9 nuclease, however mechanism for quick and efficient termination of activity of other RNA-guided nucleases, such as Cpf1, have not yet been developed. See, Zou et al. Mol Cell. (2021) 81: 1553-1565.

Aspects of the present disclosure provide photocleavable guide RNAs (gRNAs) and methods of use thereof for effecting a genetic modification (e.g., mutation) in the genome of a cell, in a controllable/regulatable manner. The photocleavable gRNAs described herein comprise a targeting domain, a CRISPR RNA (crRNA) sequence for a CRISPR/Cas nuclease, and at least one photocleavable moiety. Upon contact with light of a suitable wavelength, the photocleavable moiety is cleaved, resulting cleavage of the gRNA into two or more fragments. In some embodiments, the cleaved gRNA fragments have reduced or eliminated activity (e.g., reduced or eliminated binding to a CRISPR/Cas nuclease, and/or reduced or eliminated binding or recruitment to a target site sequence), and thus reduction or termination of CRISPR/Cas nuclease cleavage a the target site sequence. Also provided herein are CRISPR/Cas systems comprising a photocleavable gRNA and a CRISPR/Cas nuclease, and methods of producing CRISPR/Cas systems. Also provided herein are methods of genetic modulation of a cell to effect a genetic modification (e.g., mutation) in the genome of a cell.

The term “mutation,” as used herein, refers to a change (e.g., an insertion, deletion, inversion, or substitution) in a nucleic acid sequence as compared to a reference sequence, e.g., the corresponding sequence of a cell not having such a mutation, or the corresponding wild-type nucleic acid sequence. In some embodiments provided herein, a mutation in a gene (e.g., a target gene) results in a loss of expression of a protein encoded by the target gene in a cell harboring the mutation. In some embodiments, a mutation in a gene (e.g., a target gene) results in the expression of a variant form of a protein that is encoded by the target gene.

Some aspects of this disclosure provide compositions and methods for generating the genetically engineered cells described herein. e.g., genetically engineered cells comprising a modification in their genome, such as a modification that results in a loss of expression or regulation of a protein, or expression of a variant form of a protein. Such compositions and methods provided herein include, without limitation, suitable strategies and approaches for genetically engineering cells, e.g., by using RNA-guided nucleases, such as CRISPR/Cas nucleases, and suitable RNAs able to bind such RNA-guided nucleases and target them to a suitable target site within the genome of a cell to effect a genomic modification.

In some embodiments, a genetically engineered cell described herein is generated via genome editing technology, which includes any technology capable of introducing targeted changes, also referred to as “edits,” into the genome of a cell.

One exemplary suitable genome editing technology is “gene editing.” comprising the use of a RNA-guided nuclease, e.g., a CRISPR/Cas nuclease, to introduce targeted single- or double-stranded DNA breaks in the genome of a cell, which trigger cellular repair mechanisms, such as, for example, nonhomologous end joining (NHEJ), microhomology-mediated end joining (MMEJ, also sometimes referred to as “alternative NHEJ” or “alt-NHEJ”), or homology-directed repair (HDR) that typically result in an altered nucleic acid sequence (e.g., via nucleotide or nucleotide sequence insertion, deletion, inversion, or substitution) at or immediately proximal to the site of the nuclease cut. See, e.g., Yeh et al. Nat. Cell. Biol. (2019) 21: 1468-1478; e.g., Hsu et al. Cell (2014) 157: 1262-1278; Jasin et al. DNA Repair (2016) 44: 6-16; Sfeir et al. Trends Biochem. Sci. (2015) 40: 701-714.

Another exemplary suitable genome editing technology is “base editing,” which includes the use of a base editor, e.g., a nuclease-impaired or partially nuclease-impaired RNA-guided CRISPR/Cas protein fused to a deaminase that targets and deaminates a specific nucleobase, e.g., a cytosine or adenosine nucleobase of a C or A nucleotide, which, via cellular mismatch repair mechanisms, results in a change from a C to a T nucleotide, or a change from an A to a G nucleotide. See, e.g., Komor et al. Nature (2016) 533: 420-424; Rees et al. Nat. Rev. Genet. (2018) 19(12): 770-788; Anzalone et al. Nat. Biotechnol. (2020) 38: 824-844.

Yet another exemplary suitable genome editing technology includes “prime editing.” which includes the introduction of new genetic information, e.g., an altered nucleotide sequence, into a specifically targeted genomic site using a catalytically impaired or partially catalytically impaired RNA-guided nuclease, e.g., a CRISPR/Cas nuclease, fused to an engineered reverse transcriptase (RT) domain. The Cas/RT fusion is targeted to a target site within the genome by a guide RNA that also comprises a nucleic acid sequence encoding the desired edit, and that can serve as a primer for the RT. See, e.g., Anzalone et al. Nature (2019) 576 (7785): 149-157.

The use of genome editing technology typically features the use of a suitable RNA-guided nuclease, which, in some embodiments, e.g., for base editing or prime editing, may be catalytically impaired, or partially catalytically impaired. Examples of suitable RNA-guided nucleases include CRISPR/Cas nucleases. For example, in some embodiments, a suitable RNA-guided nuclease for use in the methods of genetically engineering cells provided herein is a Cas9 nuclease, e.g., an spCas9 or an saCas9 nuclease. For another example, in some embodiments, a suitable RNA-guided nuclease for use in the methods of genetically engineering cells provided herein is a Cas12 nuclease, e.g., a Cas12a nuclease (also referred to as “Cpf1”). Exemplary suitable Cas12 nucleases include, without limitation, AsCas 12a, FnCas12a, LbCas12a, PaCas12a, other Cas12a orthologs, and Cas12a derivatives, such as the MAD7 system (MAD7™, Inscripta, Inc.), or the Alt-R Cas12a (Cpf1) Ultra nuclease (Alt-R® Cas12a Ultra; Integrated DNA Technologies, Inc.). See, e.g., Gill et al. LIPSCOMB 2017. In United States: Inscripta Inc.; Price et al. Biotechnol. Bioeng. (2020) 117(60): 1805-1816; PCT Publication Nos. WO 2016/166340; WO 2017/155407; WO 2018/083128; WO 2016/205711; WO 2017/035388; WO 2017/184768; WO2019/118516; WO2017/184768; WO 2018/098383; WO 2020/146297; and WO 2020/172502.

In some embodiments, a genetically engineered cell described herein is generated by targeting an RNA-guided nuclease, e.g., a CRISPR/Cas nuclease, such as, for example, a Cas9 nuclease or a Cas 12a nuclease, to a suitable target site in the genome of the cell, under conditions suitable for the RNA-guided nuclease to bind the target site and cut the genomic DNA of the cell. A suitable RNA-guided nuclease can be targeted to a specific target site within the genome by a suitable guide RNA (gRNA). Suitable gRNAs for targeting CRISPR/Cas nucleases according to aspects of this disclosure are provided herein and exemplary suitable gRNAs (i.e., photocleavable gRNAs) are described in more detail elsewhere herein.

In some embodiments, any of the photocleavable gRNAs described herein may be complexed with a suitable CRISPR/Cas nuclease. Exemplary suitable nucleases include, for example, Cas 9 nuclease and Cas12a (Cpf1) nucleases.

Various Cas9 nucleases are suitable for use with the photocleavable gRNAs provided herein to effect genome editing according to aspects of this disclosure, e.g., to create a genomic modification in a target genetic loci. Typically, the CRISPR/Cas nuclease and the gRNA are provided in a form and under conditions suitable for the formation of a nuclease/gRNA complex (i.e., a CRISPR system), that targets a target site on the genome of the cell. In some embodiments, a CRISPR/Cas nuclease is used that exhibits a desired PAM specificity to target the Cas/gRNA complex to a desired target site sequence in a genetic loci. Example target domains and corresponding gRNA targeting domain sequences are provided herein.

In some embodiments, nuclease/gRNA complex is formed, e.g., in vitro, and a target cell is contacted with the nuclease/gRNA complex, e.g., via electroporation of the nuclease/gRNA complex into the cell. In some embodiments, the cell is contacted with CRISPR/Cas protein and gRNA separately, and the nuclease/gRNA complex is formed within the cell. In some embodiments, the cell is contacted with a nucleic acid, e.g., a DNA or RNA, encoding the CRISPR/Cas protein, and/or with a nucleic acid encoding the gRNA, or both.

In some embodiments, a Cas nuclease is used that belongs to class 2 type V of Cas nucleases. Class 2 type V Cas nucleases can be further categorized as type V-A, type V-B, type V-C, and type V-U. See, e.g., Stella et al. Nature Structural & Molecular Biology (2017). In some embodiments, the Cas nuclease is a type V-B Cas endonuclease, such as a C2c1. See, e.g., Shmakov et al. Mol Cell (2015) 60: 385-397. In some embodiments, the Cas nuclease used in the methods of genome editing provided herein is a type V-A Cas endonuclease, such as a Cpf1 (Cas12a) nuclease. See, e.g., Strohkendl et al. Mol. Cell (2018) 71: 1-9.

In some embodiments, genetically engineered cells as provided herein are generated using a suitable genome editing technology, wherein the genome editing technology is characterized by the use of a Cas12a (Cpf1) nuclease.

In some embodiments, a CRISPR/Cas nuclease used in the methods of genome editing provided herein is a Cas 12a (Cpf1) nuclease derived from Provetella spp. (e.g., Provetella ihumii (PiCas12a/PiCpf1) or Provetella disiens (PdCas12a/PdCpf1) or Francisella spp. (Francisella novicida (FnCas12a/FnCpf1)), Acidaminococcus sp. (AsCas 12a/AsCpf1), Lachnospiraceae bacterium (LpCas12a/LpCpf1), or Eubacterium rectale (ErCas12a/ErCpf1). In some embodiments, the CRISPR/Cas nuclease is MAD7.

In some embodiments, genetically engineered cells as provided herein are generated using a suitable genome editing technology, wherein the genome editing technology is characterized by the use of a Cas9 nuclease. In some embodiments, the Cas9 molecule is of, or derived from, Streptococcus pyogenes (SpCas9), Staphylococcus aureus (SaCas9), or Streptococcus thermophilus (StCas9). Additional suitable Cas9 molecules include those of, or derived from, Neisseria meningitidis (NmCas9), Acidovorax avenae, Actinobacillus pleuropneumoniae, Actinobacillus succinogenes, Actinobacillus suis, Actinomyces sp., cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni (CjCas9), Campylobacter lari, Candidatus Puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria meningitidis, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tistrella mobilis, Treponema sp., or Verminephrobacter eiseniae. In some embodiments, catalytically impaired, or partially impaired, variants of such Cas9 nucleases may be used. Additional suitable Cas9 nucleases, and nuclease variants, will be apparent to those of skill in the art based on the present disclosure. The disclosure is not limited in this respect.

In some embodiments, the Cas nuclease is a naturally occurring Cas molecule. In some embodiments, the Cas nuclease is an engineered, altered, or modified Cas molecule that differs, e.g., by at least one amino acid residue, from a reference sequence, e.g., the most similar naturally occurring Cas9 molecule or a sequence of Table 50 of PCT Publication No. WO2015/157070, which is herein incorporated by reference in its entirety.

Both naturally occurring and modified variants of CRISPR/Cas nucleases are suitable for use according to aspects of this disclosure. For example, dCas or nickase variants, Cas variants having altered PAM specificities, and Cas variants having improved nuclease activities are embraced by some embodiments of this disclosure.

Some features of some exemplary, non-limiting suitable Cas nucleases are described in more detail herein, without wishing to be bound to any particular theory.

A naturally occurring Cas9 nuclease typically comprises two lobes: a recognition (REC) lobe and a nuclease (NUC) lobe; each of which further comprises domains described, e.g., in PCT Publication No. WO2015/157070, e.g., in FIGS. 9A-9B therein (which application is incorporated herein by reference in its entirety).

The REC lobe comprises the arginine-rich bridge helix (BH), the REC1 domain, and the REC2 domain. The REC lobe appears to be a Cas9-specific functional domain. The BH domain is a long alpha helix and arginine rich region and comprises amino acids 60-93 of the sequence of S. pyogenes Cas9. The REC1 domain is involved in recognition of the repeat:anti-repeat duplex. e.g., of a gRNA or a tracrRNA. The REC1 domain comprises two REC1 motifs at amino acids 94 to 179 and 308 to 717 of the sequence of S. pyogenes Cas9. These two REC1 domains, though separated by the REC2 domain in the linear primary structure, assemble in the tertiary structure to form the REC1 domain. The REC2 domain, or parts thereof, may also play a role in the recognition of the repeat: anti-repeat duplex. The REC2 domain comprises amino acids 180-307 of the sequence of S. pyogenes Cas9.

The NUC lobe comprises the RuvC domain (also referred to herein as RuvC-like domain), the HNH domain (also referred to herein as HNH-like domain), and the PAM-interacting (PI) domain. The RuvC domain shares structural similarity to retroviral integrase superfamily members and cleaves a single strand, e.g., the non-complementary strand of the target nucleic acid molecule. The RuvC domain is assembled from the three split RuvC motifs (RuvC I, RuvCII, and RuvCIII, which are often commonly referred to in the art as RuvCI domain, or N-terminal RuvC domain, RuvCII domain, and RuvCIII domain) at amino acids 1-59, 718-769, and 909-1098, respectively, of the sequence of S. pyogenes Cas9. Similar to the REC1 domain, the three RuvC motifs are linearly separated by other domains in the primary structure, however in the tertiary structure, the three RuvC motifs assemble and form the RuvC domain. The HNH domain shares structural similarity with HNH endonucleases, and cleaves a single strand, e.g., the complementary strand of the target nucleic acid molecule. The HNH domain lies between the RuvC II-III motifs and comprises amino acids 775-908 of the sequence of S. pyogenes Cas9. The PI domain interacts with the PAM of the target nucleic acid molecule and comprises amino acids 1099-1368 of the sequence of S. pyogenes Cas9.

Crystal structures have been determined for naturally occurring bacterial Cas9 nucleases (see, e.g., Jinek et al., Science, 343(6176): 1247997, 2014) and for S. pyogenes Cas9 with a guide RNA (e.g., a synthetic fusion of crRNA and tracrRNA) (Nishimasu et al., Cell, 156:935-949, 2014; and Anders et al., Nature, 2014, doi: 10.1038/nature13579).

In some embodiments, a Cas9 molecule described herein exhibits nuclease activity that results in the introduction of a double strand DNA break in or directly proximal to a target site. In some embodiments, the Cas9 molecule has been modified to inactivate one of the catalytic residues of the endonuclease. In some embodiments, the Cas9 molecule is a nickase and produces a single stranded break. See, e.g., Dabrowska et al. Frontiers in Neuroscience (2018) 12(75). It has been shown that one or more mutations in the RuvC and HNH catalytic domains of the enzyme may improve Cas9 efficiency. See. e.g., Sarai et al. Currently Pharma. Biotechnol. (2017) 18(13). In some embodiments, the Cas9 molecule is fused to a second domain, e.g., a domain that modifies DNA or chromatin, e.g., a deaminase or demethylase domain. In some such embodiments, the Cas9 molecule is modified to eliminate its endonuclease activity.

In some embodiments, a Cas nuclease or a Cas/gRNA complex described herein is administered together with a template for homology directed repair (HDR). In some embodiments, a Cas nuclease or a Cas/gRNA complex described herein is administered without a HDR template.

In some embodiments, a Cas9 nuclease is used that is modified to enhance specificity of the enzyme (e.g., reduce off-target effects, maintain robust on-target cleavage). In some embodiments, the Cas9 molecule is an enhanced specificity Cas9 variant (e.g., eSPCas9). See, e.g., Slaymaker et al. Science (2016) 351 (6268): 84-88. In some embodiments, the Cas9 molecule is a high fidelity Cas9 variant (e.g., SpCas9-HF1). See, e.g., Kleinstiver et al. Nature (2016) 529: 490-495.

Various CRISPR/Cas nucleases, which may also be referred to as Cas nucleases, are known in the art and may be obtained from various sources and/or engineered/modified to modulate one or more activities or specificities of the enzymes. PAM sequence preferences and specificities of suitable Cas nucleases, e.g., suitable Cas9 nucleases, such as, for example, spCas9 and saCas9 are known in the art. In some embodiments, the Cas nuclease has been engineered/modified to recognize one or more PAM sequence. In some embodiments, the Cas nuclease has been engineered/modified to recognize one or more PAM sequence that is different than the PAM sequence the Cas nuclease recognizes without engineering/modification. In some embodiments, the Cas nuclease has been engineered/modified to reduce off-target activity of the enzyme.

In some embodiments, a Cas nuclease is used that is modified further to alter the specificity of the endonuclease activity (e.g., reduce off-target cleavage, decrease the endonuclease activity or lifetime in cells, increase homology-directed recombination and reduce non-homologous end joining). See, e.g., Komor et al. Cell (2017) 168: 20-36. In some embodiments, a Cas nuclease is used that is modified to alter the PAM recognition or preference of the endonuclease. For example, SpCas9 recognizes the PAM sequence NGG, whereas some variants of SpCas9 comprising one or more modifications (e.g., VQR SpCas9, EQR SpCas9, VRER SpCas9) may recognize variant PAM sequences, e.g., NGA, NGAG, and/or NGCG. For another example, SaCas9 recognizes the PAM sequence NNGRRT, whereas some variants of SaCas9 comprising one or more modifications (e.g., KKH SaCas9) may recognize the PAM sequence NNNRRT. In another example, FnCas9 recognizes the PAM sequence NNG, whereas a variant of the FnCas9 comprises one or more modifications (e.g., RHA FnCas9) may recognize the PAM sequence YG. In another example, the Cas12a nuclease comprising substitution mutations S542R and K607R recognizes the PAM sequence TYCV. In another example, a Cpf1 endonuclease comprising substitution mutations S542R, K607R, and N552R recognizes the PAM sequence TATV. See, e.g., Gao et al. Nat. Biotechnol. (2017) 35(8): 789-792.

In some embodiments, a base editor is used to create a genomic modification in a cell. Base editors typically comprise a catalytically inactive or partially inactive Cas nuclease fused to a functional domain, e.g., a deaminase domain. See, e.g., Eid et al. Biochem. J. (2018) 475(11): 1955-1964; Rees et al. Nature Reviews Genetics (2018) 19:770-788. In some embodiments, a catalytically inactive Cas nuclease is referred to as “dead Cas” or “dCas.” In some embodiments, the endonuclease comprises a dCas fused to an adenine base editor (ABE), for example an ABE evolved from the RNA adenine deaminase TadA. In some embodiments, the endonuclease comprises a dCas fused to cytidine deaminase enzyme (e.g., APOBEC deaminase, pmCDA1, activation-induced cytidine deaminase (AID)). In some embodiments, the catalytically inactive Cas molecule has reduced activity and is, e.g., a nickase.

Examples of suitable base editors include, without limitation, BE1, BE2, BE3, HF-BE3, BE4, BE4max, BE4-Gam. YE1-BE3, EE-BE3, YE2-BE3, YEE-CE3, VQR-BE3, VRER-BE3, SaBE3, SaBE4, SaBE4-Gam, Sa(KKH)-BE3, Target-AID, Target-AID-NG, xBE3, eA3A-BE3, BE-PLUS, TAM, CRISPR-X, ABE7.9. ABE7.10, ABE7.10*, xABE, ABESa, VQR-ABE, VRER-ABE, Sa(KKH)-ABE, and CRISPR-SKIP. Additional examples of base editors can be found, for example, in US Publication No. 2018/0312825A1. US Publication No.

2018/0312828A1, and PCT Publication No. WO 2018/165629A1, which are incorporated by reference herein in their entireties.

Some aspects of this disclosure provide photocleavable guide RNAs that are suitable to target an RNA-guided nuclease, e.g. as provided herein, to a suitable target site in the genome of a cell. In some embodiments, the photocleavable gRNA effects a modification in the genome of the cell (e.g., insertion, mutation, deletion). Such modifications may result in a loss of expression and/or regulation of a protein encoded by a gene, or expression of a variant form of a protein encoded by a genet that is targeted by the gRNA.

The terms “guide RNA” and “gRNA” are used interchangeably herein and refer to a nucleic acid, typically an RNA, that is bound by an RNA-guided nuclease and promotes the specific targeting or homing of the RNA-guided nuclease to a target nucleic acid, e.g., a target site within the genome of a cell. A gRNA typically comprises at least two domains: a “binding domain,” also sometimes referred to as “gRNA scaffold” or “gRNA backbone” that mediates binding to an RNA-guided nuclease (also referred to as the “binding domain”), and a “targeting domain” that mediates the targeting of the gRNA-bound RNA-guided nuclease to a target site. Some gRNAs comprise additional domains, e.g., complementarity domains, or stem-loop domains. The structures and sequences of naturally occurring gRNA binding domains and engineered variants thereof are well known to those of skill in the art. Some suitable gRNAs are unimolecular, comprising a single nucleic acid sequence, while other suitable gRNAs comprise two sequences (e.g., a crRNA and tracrRNA sequence).

Aspects of the present disclosure relate to “photocleavable gRNAs,” which refers to guide RNAs that contain a photocleavable moiety and are thereby cleaved upon contact with light of a suitable wavelength. Upon photocleavage, the gRNA is cut into at least two fragments.

In some embodiments, the photocleavable gRNA comprises a targeting domain, a CRISPR RNA (crRNA) sequence (i.e., a gRNA scaffold) that binds and recruits a CRISPR/Cas nuclease, and at least one photocleavable moiety.

Suitable gRNAs for use with CRISPR/Cas nucleases, such as Cas12a nucleases, typically comprise a single RNA molecule, as the naturally occurring Cas12a guide RNA comprises a single RNA molecule. A suitable gRNA may thus be unimolecular (having a single RNA molecule), sometimes referred to herein as single guide RNAs (sgRNAs), or modular (comprising more than one, and typically two, separate RNA molecules). Some exemplary suitable Cas12a gRNA scaffold sequences are provided herein, and additional suitable gRNA scaffold sequences will be apparent to the skilled artisan based on the present disclosure.

In some embodiments, e.g., in some embodiments where a Cas12a nuclease is used, a gRNA, may comprise, from 5′ to 3′:
a CRISPR RNA (crRNA) sequence for a CRISPR/Cas nuclease, containing:
   a proximal domain;
   a first complementarity domain;
   a linking domain; and
   a second complementarity domain (which is complementary to the first complementarity domain); and
a targeting domain corresponding to a target site sequence.

Some exemplary suitable Cas9 gRNA scaffold sequences are provided herein, and additional suitable gRNA scaffold sequences will be apparent to the skilled artisan based on the present disclosure. Such additional suitable scaffold sequences include, without limitation, those recited in Jinek, et al. Science (2012) 337(6096):816-821, Ran, et al. Nature Protocols (2013) 8:2281-2308, PCT Publication No. WO2014/093694, and PCT Publication No. WO2013/176772.

For example, the binding domains of naturally occurring spCas9 gRNA typically comprise two RNA molecules, the crRNA (partially) and the tracrRNA. Variants of spCas9 gRNAs that comprise only a single RNA molecule including both crRNA and tracrRNA sequences, covalently bound to each other, e.g., via a tetraloop or via click-chemistry type covalent linkage, have been engineered and are commonly referred to as “single guide RNA” or “sgRNA.” A gRNA suitable for targeting a target site may comprise a number of domains. In some embodiments, e.g., in some embodiments where a Cas9 nuclease is used, a unimolecular sgRNA, may comprise, from 5′ to 3′:
a targeting domain corresponding to a target site sequence in a target locus;
a first complementarity domain;
a linking domain;
a second complementarity domain (which is complementary to the first complementarity domain);
a proximal domain; and
optionally, a tail domain; and
at least one photocleavable moiety.

Each of these domains is now described in more detail.

A gRNA as provided herein typically comprises a targeting domain that binds to a target site in the genome of a cell. The target site is typically a double-stranded DNA sequence comprising the PAM sequence and, on the same strand as, and directly adjacent to, the PAM sequence, the target domain. The targeting domain of the gRNA typically comprises an RNA sequence that corresponds to the target domain sequence in that it resembles the sequence of the target domain, sometimes with one or more mismatches, but typically comprises an RNA instead of a DNA sequence. The targeting domain of the gRNA thus base-pairs (in full or partial complementarity) with the sequence of the double-stranded target site that is complementary to the sequence of the target domain, and thus with the strand complementary to the strand that comprises the PAM sequence. It will be understood that the targeting domain of the gRNA typically does not include the PAM sequence. It will further be understood that the location of the PAM may be 5′ or 3′ of the target domain sequence, depending on the nuclease employed. For example, the PAM is typically 3′ of the target domain sequences for Cas9 nucleases, and 5′ of the target domain sequence for Cas12a nucleases. For an illustration of the location of the PAM and the mechanism of gRNA binding a target site, see, e.g., FIG. 1 of Vanegas et al., Fungal Biol Biotechnol. 2019; 6: 6, which is incorporated by reference herein. For additional illustration and description of the mechanism of gRNA targeting an RNA-guided nuclease to a target site, see Fu Y et al, Nat Biotechnol 2014 (doi: 10.1038/nbt.2808) and Sternberg S H et al., Nature 2014 (doi: 10.1038/naturel3011), both incorporated herein by reference.

The targeting domain may comprise a nucleotide sequence that corresponds to the sequence of the target domain, i.e., the DNA sequence directly adjacent to the PAM sequence (e.g., 5′ of the PAM sequence for Cas9 nucleases, or 3′ of the PAM sequence for Cas12a nucleases). The targeting domain sequence typically comprises between 17 and 30 nucleotides and corresponds fully with the target domain sequence (i.e., without any mismatch nucleotides), or may comprise one or more, but typically not more than 4, mismatches. As the targeting domain is part of an RNA molecule, the gRNA, it will typically comprise ribonucleotides, while the DNA targeting domain will comprise deoxyribonucleotides.

The structure of a typical Cas12a gRNA can be found, for example in FIG. 1 of Zetsche et al. Cell (2015) 163(3): 759-771, which is incorporated by reference herein in its entirety. An exemplary illustration of a Cas12a target site, comprising a 22 nucleotide target domain, and a TTN PAM sequence, as well as of a gRNA comprising a targeting domain that fully corresponds to the target domain (and thus base-pairs with full complementarity with the DNA strand complementary to the strand comprising the target domain and PAM) is provided below:

```
                          [PAM] [ target domain (DNA) ]
                    5′-T-T-N-N-N-N-N-N-N-N-N-N-N-N-N-N-N-N-N-N-N-N-N-N-N-3′ (DNA)
                    3′-A-A-N-N-N-N-N-N-N-N-N-N-N-N-N-N-N-N-N-N-N-N-N-N-N-5′ (DNA)
                                      | | | | | | | | | | | | | | |
5′-[gRNA scaffold]-N-N-N-N-N-N-N-N-N-N-N-N-N-N-N-N-N-N-N-N-N-N-3′ (RNA)
    [binding domain] [ targeting domain (RNA) ]
```

In some embodiments, the Cas12a PAM sequence is 5′-T-T-T-V-3′. In some embodiments, the Cas12a PAM sequence is 5′-T-T-V-3′.

An exemplary illustration of a Cas9 target site, comprising a 22 nucleotide target domain, and an NGG PAM sequence, as well as of a gRNA comprising a targeting domain that fully corresponds to the target domain (and thus base-pairs with full complementarity with the DNA strand complementary to the strand comprising the target domain and PAM) is provided below:

```
      [ target domain (DNA) ] [PAM]
5′-N-N-N-N-N-N-N-N-N-N-N-N-N-N-N-N-N-N-N-N-N-N-N-G-G-3′ (DNA)
3′-N-N-N-N-N-N-N-N-N-N-N-N-N-N-N-N-N-N-N-N-N-N-N-C-C-5′ (DNA)
      | | | | | | | | | | | | | | |
5′-N-N-N-N-N-N-N-N-N-N-N-N-N-N-N-N-N-N-N-N-N-N-[gRNA scaffold]-3′ (RNA)
      [ targeting domain (RNA) ] [binding domain]
```

While not wishing to be bound by theory, at least in some embodiments, it is believed that the length and complementarity of the targeting domain with the target sequence contributes to specificity of the interaction of the gRNA/Cas molecule complex with a target nucleic acid. In some embodiments, the targeting domain of a gRNA provided herein is 5 to 50 nucleotides in length. In some embodiments, the targeting domain is 15 to 25 nucleotides in length. In some embodiments, the targeting domain is 18 to 22 nucleotides in length. In some embodiments, the targeting domain is 19-21 nucleotides in length. In some embodiments, the targeting domain is 15 nucleotides in length. In some embodiments, the targeting domain is 16 nucleotides in length. In some embodiments, the targeting domain is 17 nucleotides in length. In some embodiments, the targeting domain is 18 nucleotides in length. In some embodiments, the targeting domain is 19 nucleotides in length. In some embodiments, the targeting domain is 20 nucleotides in length. In some embodiments, the targeting domain is 21 nucleotides in length. In some embodiments, the targeting domain is 22 nucleotides in length. In some embodiments, the targeting domain is 23 nucleotides in length. In some embodiments, the targeting domain is 24 nucleotides in length. In some embodiments, the targeting domain is 25 nucleotides in length. In some embodiments, the targeting domain fully corresponds, without mismatch, to a target domain sequence provided herein, or a part thereof. In some embodiments, the targeting domain of a gRNA provided herein comprises 1 mismatch relative to a target domain sequence provided herein. In some embodiments, the targeting domain comprises 2 mismatches relative to the target domain sequence. In some embodiments, the target domain comprises 3 mismatches relative to the target domain sequence.

In some embodiments, a targeting domain comprises a core domain and a secondary targeting domain, e.g., as described in PCT Publication No. WO2015/157070, which is incorporated by reference in its entirety. In some embodiments, the core domain comprises about 8 to about 13 nucleotides from the 3′ end of the targeting domain (e.g., the most 3′ 8 to 13 nucleotides of the targeting domain). In some embodiments, the secondary domain is positioned 5′ to the core domain. In some embodiments, the core domain corresponds fully with the target domain sequence, or a part thereof. In other embodiments, the core domain may comprise one or more nucleotides that are mismatched with the corresponding nucleotide of the target domain sequence.

In some embodiments, e.g., in some embodiments where a Cas9 gRNA is provided, the gRNA comprises a first complementarity domain and a second complementarity domain, wherein the first complementarity domain is complementary with the second complementarity domain, and, at least in some embodiments, has sufficient complementarity to the second complementarity domain to form a duplexed region under at least some physiological conditions. In some embodiments, the first complementarity domain is 5 to 30 nucleotides in length. In some embodiments, the first complementarity domain comprises 3 subdomains, which, in the 5′ to 3′ direction are: a 5′ subdomain, a central subdomain, and a 3′ subdomain. In some embodiments, the 5′ subdomain is 4 to 9, e.g., 4, 5, 6, 7, 8 or 9 nucleotides in length. In some embodiments, the central subdomain is 1, 2, or 3, e.g., 1, nucleotide in length. In some embodiments, the 3′ subdomain is 3 to 25, e.g., 4 to 22, 4 to 18, or 4 to 10, or 3, 4, 5, 6, 7, 8, 9, 10, 11, 12, 13, 14, 15, 16, 17, 18, 19, 20, 21, 22, 23, 24, or 25 nucleotides in length. The first complementarity domain can share homology with, or be derived from, a naturally occurring first complementarity domain. In an embodiment, it has at least 50% homology with a S. pyogenes, S. aureus or S. thermophilus, first complementarity domain.

The sequence and placement of the above-mentioned domains are described in more detail in PCT Publication No. WO 2015/157070, which is herein incorporated by reference in its entirety, including p. 88-112 therein.

A linking domain may serve to link the first complementarity domain with the second complementarity domain of a unimolecular gRNA. The linking domain can link the first and second complementarity domains covalently or non-covalently. In some embodiments, the linkage is covalent. In some embodiments, the linking domain is, or comprises, a covalent bond interposed between the first complementarity domain and the second complementarity domain. In some embodiments, the linking domain comprises one or more, e.g., 2, 3, 4, 5, 6, 7, 8, 9, or 10 nucleotides. In some embodiments, the linking domain comprises at least one non-nucleotide bond, e.g., as disclosed in PCT Publication No. WO 2018/126176, the entire contents of which are incorporated herein by reference.

In some embodiments, the second complementarity domain of the targeting domain is complementary, at least in part, with the first complementarity domain, and in an embodiment, has sufficient complementarity to the second complementarity domain to form a duplexed region under at least some physiological conditions. In some embodiments, the second complementarity domain can include a sequence that lacks complementarity with the first complementarity domain, e.g., a sequence that loops out from the duplexed region. In some embodiments, the second complementarity domain is 5 to 27 nucleotides in length. In some embodiments, the second complementarity domain is longer than the first complementarity region. In an embodiment, the complementary domain is 5, 6, 7, 8, 9, 10, 11, 12, 13, 14, 15, 16, 17, 18, 19, 20, 21, 22, 23, 24 or 25 nucleotides in length. In some embodiments, the second complementarity domain comprises 3 subdomains, which, in the 5′ to 3′ direction are: a 5′ subdomain, a central subdomain, and a 3′ subdomain. In some embodiments, the 5′ subdomain is 3 to 25, e.g., 4 to 22, 4 to 18, or 4 to 10, or 3, 4, 5, 6, 7, 8, 9, 10, 11, 12, 13, 14, 15, 16, 17, 18, 19, 20, 21, 22, 23, 24, or 25 nucleotides in length. In some embodiments, the central subdomain is 1, 2, 3, 4 or 5. e.g., 3, nucleotides in length. In some embodiments, the 3′ subdomain is 4 to 9, e.g., 4, 5, 6, 7, 8 or 9 nucleotides in length. In some embodiments, the 5′ subdomain and the 3′ subdomain of the first complementarity domain, are respectively, complementary, e.g., fully complementary, with the 3′ subdomain and the 5′ subdomain of the second complementarity domain.

In some embodiments, the proximal domain is 5 to 20 nucleotides in length. In some embodiments, the proximal domain can share homology with or be derived from a naturally occurring proximal domain. In an embodiment, it has at least 50% homology with a proximal domain from S. pyogenes, S. aureus, or S. thermophilus.

A broad spectrum of tail domains are suitable for use in gRNAs. In some embodiments, the tail domain is 0 (absent). 1, 2, 3, 4, 5, 6, 7, 8, 9, or 10 nucleotides in length. In some embodiments, the tail domain nucleotides are from or share homology with a sequence from the 5′ end of a naturally occurring tail domain. In some embodiments, the tail domain includes sequences that are complementary to each other and which, under at least some physiological conditions, form a duplexed region. In some embodiments, the tail domain is absent or is 1 to 50 nucleotides in length. In some embodiments, the tail domain can share homology with or be derived from a naturally occurring proximal tail domain. In some embodiments, the tail domain has at least 50% homology/identity with a tail domain from S. pyogenes, S. aureus or S. thermophilus. In some embodiments, the tail domain includes nucleotides at the 3′ end that are related to the method of in vitro or in vivo transcription.

In some embodiments, a gRNA provided herein comprises:
a first strand comprising, e.g., from 5′ to 3′:
   a targeting domain (which corresponds to a target domain in a target locus); and
   a first complementarity domain;
a second strand, comprising, e.g., from 5′ to 3′:
   optionally, a 5′ extension domain;
   a second complementarity domain;
   a proximal domain; and
   optionally, a tail domain;
and at least one photocleavable moiety.

As used herein, the term “photocleavable moiety” refers to a moiety that is present between two nucleotides (e.g., a bond) that is capable to being cleaved upon contact with light of a suitable wavelength. In some embodiments, the photocleavable moiety is a photocleavable bond. In some embodiments, the photocleavable moiety links two nucleotides and is capable of being cleaved upon contact with light of a suitable wavelength. Upon cleavage of the photocleavable moiety, the gRNA is cut into at least two fragments.

In some embodiments, a photocleavable moiety comprises a photocleavable group. In some embodiments, a photocleavable moiety comprises a nucleotide (e.g., A, G. C. T, or U) comprising a photocleavable group. As used herein, a photocleavable group is a compound that absorbs light at a specific wavelength and undergoes photochemistry, such that, when the photocleavable group is part of an oligonucleotide (e.g., a gRNA), the photochemistry may result in cleavage of the oligonucleotide into at least two fragments. In some embodiments, the photocleavable group undergoes photochemistry where at least 50% (e.g., at least 60, 70, 80, 90, 95, 99, or 100%) of photochemical reactions proceeding from excitation events yield photocleavage products (i.e., products wherein the gRNA has broken into a plurality of nucleic acids). Many photocleavable groups are known in the art.

Generally, a suitable photocleavable group has one or more of the following characteristics: one or more strong absorption peaks (e.g., at wavelengths above 300 nm to avoid damaging the biological system (e.g., a cell) and/or below 400 nm or 380 nm to minimize reactions from visible light); a reasonable (e.g., high) quantum yield of cleavage photo-product; low background photo-activity in the absence of radiation of peak absorbance; soluble; chemically stable under physiological and/or laboratory conditions; biocompatible photochemical byproducts that do not react or minimally/inconsequentially react with components of the biological system (e.g., the cell); and/or photochemical byproducts that do not absorb or absorb at a reasonably low level at the peak absorption wavelength of the photocleavable group.

As described herein, the photocleavable gRNAs contain at least one photocleavable moieties. In some embodiments, the photocleavable gRNAs comprises 1, 2, 3, 4, 5, 6, 7, 8, 9, 10 or more photocleavable moieties.

The location of a photocleavable moiety within a gRNA may be identified based on the particular domain or region of the gRNA in which the linker occurs, relative to the 5′ or 3′ terminus of the gRNA, relative to the 5′ or 3′ terminus of a particular domain or region of the gRNA, relative to a position in the target site sequence, relative to the PAM sequence associate with the target site sequence, and/or relative the genomic loci. The location of the one or more photocleavable moiety within a gRNA may be based on factors such as being non-disruptive of the CRISPR/Cas nuclease activity in the absence of light (e.g., allows the CRISPR system to maintain on-target cleavage and not increase off-target effects), the ability to be cleaved in the presence of light (efficiency of cleavage in the location) including accessibility of the position to light, and inhibition efficiency (e.g. photocleavage at the position effectively eliminates or reduces cleavage at the target site sequence).

In some embodiments, the photocleavable moiety replaces a nucleotide of the gRNA, such as a nucleotide of the targeting domain corresponding to the target sequence, or a nucleotide of the crRNA domain. In some embodiments, the photocleavable moiety replaces a nucleotide of the gRNA, such as a nucleotide of the targeting domain corresponding to the target sequence, or a nucleotide of the crRNA domain as compared to a gRNA that targets the target site sequence and does not include a photocleavable moiety. Alternatively, in some embodiments, the photocleavable moiety is inserted between two nucleotides of the gRNA and therefore does not alter the nucleotide sequence of the gRNA.

In general, a photocleavable moiety is said to be “in” or “within” a particular domain or region of the gRNA if the moiety is between two nucleotides considered to be within the domain or region. In some embodiments, the photocleavable moiety is present in the targeting domain of the gRNA. In some embodiments, the gRNA contains more than one photocleavable moiety, which may be present in the same domain or region of the gRNA, or in different domains or regions of the gRNA.

The position of the photocleavable moiety may described as following a nucleotide at a recited position. As described herein, a photocleavable moiety that is following a nucleotide at a recited position is location between the nucleotide and an adjacent second nucleotide (e.g., a second nucleotide located 3′ relative to the (first) nucleotide). Alternatively or in addition, the location of a photocleavable moiety within a gRNA may be identified based on the nucleotide position that is replaced by the photocleavable moiety. For example, a photocleavable moiety that is present at the third position (position 3) from the 5′ end of the gRNA is located between the second and fourth nucleotides, in place of the third nucleotide.

In some embodiments, a photocleavable moiety is located following the nucleotide at position 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 from the 5′ end of the targeting domain. In some embodiments, a photocleavable moiety is located following the nucleotide at position 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 from the 5′ end of the gRNA. In some embodiments, a photocleavable moiety is located following the nucleotide at any one or more of positions 9, 11, 13, 15, or 17 from the 5′ end of the targeting domain. In some embodiments, a photocleavable moiety is located following the nucleotide at any one or more of positions 9, 11, 13, 15, or 17 from the 5′ end of the gRNA.

In some embodiments, a photocleavable moiety is located between nucleotide positions 1 and 3, 2 and 4, 3 and 5, 4 and 6, 5 and 7, 6 and 8, 7 and 9, 8 and 10, 9 and 11, 10 and 12, 11 and 13, 12 and 14, 13 and 15, 14 and 16, 15 and 17, 16 and 18, 17 and 19, 18 and 20, 19 and 21, or 20 and 22 from the 5′ end of the targeting domain. In some embodiments, a photocleavable moiety is located between nucleotide positions 1 and 3, 2 and 4, 3 and 5, 4 and 6, 5 and 7, 6 and 8, 7 and 9, 8 and 10, 9 and 11, 10 and 12, 11 and 13, 12 and 14, 13 and 15, 14 and 16, 15 and 17, 16 and 18, 17 and 19, 18 and 20, 19 and 21, or 20 and 22 from the 5′ end of the gRNA.

In some embodiments, a photocleavable moiety is located within 1, 2, 3, 4, 5, 6, 7, 8, 9, 10, 11, 12, 13, 14, 15, 16, 17, 18, 19, or 20 nucleotides from the 3′ end of the targeting domain. In some embodiments, a photocleavable moiety is located within the targeting domain following a position corresponding to the nucleotide at a position 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides adjacent to the PAM associated with the target site sequence. In some embodiments, a photocleavable moiety is located within the targeting domain following a position corresponding to the nucleotide at one or more of positions 9, 11, 13, 15, or 17 nucleotides adjacent to the PAM. In some embodiments, a photocleavable moiety is located within the targeting domain between nucleotide positions 1 and 3, 2 and 4, 3 and 5, 4 and 6, 5 and 7, 6 and 8, 7 and 9, 8 and 10, 9 and 11, 10 and 12, 11 and 13, 12 and 14, 13 and 15, 14 and 16, 15 and 17, 16 and 18, 17 and 19, 18 and 20, 19 and 21, 20 and 22, 21 and 23, 22 and 24, 23 and 25, 24 and 26, 25 and 27, 26 and 28, 27 and 29, or 28 and 30 adjacent to the PAM.

In some embodiments, a photocleavable moiety is present in the CRISPR RNA (crRNA) sequence of the gRNA. As described herein, the crRNA sequence may comprise a first complementarity domain, a linking domain, and a second complementarity domain that is complementary to the first complementarity domain, and a proximal domain. The first and second complementarity domains may form a stem loop structure (a duplexed region) and play a role in binding and recruiting the CRISPR/Cas nuclease to the target site. In some embodiments, a photocleavable moiety is present in the first complementarity domain of the crRNA. In some embodiments, a photocleavable moiety is present in the linking domain. In some embodiments, a photocleavable moiety is present in the second complementarity domain of the crRNA. In some embodiments, a photocleavable moiety is present in the proximal domain of the crRNA.

In some embodiments, a photocleavable moiety is located following the nucleotide at position 1, 2, 3, 4, 5, 6, 7, 8, 9, 10, 11, 12, 13, 14, 15, 16, 17, 18, 19, or 20 from the 5′ end of the crRNA sequence. In some embodiments, a photocleavable moiety is located following the nucleotide at position 1, 2, 3, 4, 5, 6, 7, 8, 9, 10, 11, 12, 13, 14, 15, 16, 17, 18, 19, or 20 from the 5′ end of the gRNA. In some embodiments, a photocleavable moiety is located between nucleotide positions 1 and 3, 2 and 4, 3 and 5, 4 and 6, 5 and 7, 6 and 8, 7 and 9, 8 and 10, 9 and 11, 10 and 12, 11 and 13, 12 and 14, 13 and 15, 14 and 16, 15 and 17, 16 and 18, 17 and 19, 18 and 20 from the 5′ end of the crRNA sequence.

In some embodiments, a photocleavable moiety is located within 1, 2, 3, 4, 5, 6, 7, 8, 9, 10, 11, 12, 13, 14, 15, 16, 17, 18, 19, or 20 nucleotides from the 3′ end of the crRNA sequence. In some embodiments, a photocleavable moiety is located between nucleotide positions 1 and 3, 2 and 4, 3 and 5, 4 and 6, 5 and 7, 6 and 8, 7 and 9, 8 and 10, 9 and 11, 10 and 12, 11 and 13, 12 and 14, 13 and 15, 14 and 16, 15 and 17, 16 and 18, 17 and 19, 18 and 20 from the 3′ end of the 3′ end of the crRNA sequence. In some embodiments, a photocleavable moiety is located within 1, 2, 3, 4, 5, 6, 7, 8, 9, 10, 11, 12, 13, 14, 15, 16, 17, 18, 19, or 20 nucleotides from the 3′ end of the gRNA. In some embodiments, a photocleavable moiety is located between nucleotide positions 1 and 3, 2 and 4, 3 and 5, 4 and 6, 5 and 7, 6 and 8, 7 and 9, 8 and 10, 9 and 11, 10 and 12, 11 and 13, 12 and 14, 13 and 15, 14 and 16, 15 and 17, 16 and 18, 17 and 19, 18 and 20 from the 3′ end of the gRNA.

In some embodiments, the photocleavable moiety is present between the targeting domain and the CRISPR RNA (crRNA) sequence of the gRNA. In some embodiments, the photocleavable moiety is between two domains or regions of the gRNA, referring to a linker that is between a nucleotide considered to be within a first domain or region (e.g., a crRNA domain) and a nucleotide considered to be within a second domain or region (e.g., a targeting domain). For example, the photocleavable moiety may be present between a nucleotide of the crRNA domain and a nucleotide of the targeting domain of the gRNA.

In some embodiments, a photocleavable moiety may be located between any two nucleotides of the gRNA.

Using the exemplary Cas12a gRNA structure shown below, a photocleavable moiety may be present at any one or more nucleotide positions of the targeting sequence. An exemplary suitable Cas12a crRNA sequence is provided by 5′—UAAUUUCUACUCUUGUAGAU—3′ (SEQ ID NO: 44). Additional suitable Cas12a crRNA sequences, including, but not limited to, orthologs and variants, of the Cas 12a crRNA provided by SEQ ID NO: 44, will be apparent to those of skill in the art based on the present disclosure.

Exemplary Cas12 gRNAs comprising a crRNA domain and a targeting domain of the general structure 5′-[cRNA]-[targeting domain]-3′ may comprise the crRNA sequence provided by SEQ ID NO: 44 and a targeting domain between 17 to 25 nucleotides as shown below.

(SEQ ID NO: 55)
5′-UAAUUUCUACUCUUGUAGAU-[N]₁₇₋₂₅-3′
(SEQ ID NO: 45)
5′-UAAUUUCUACUCUUGUAGAU-N1-N2-N3-N4-N5-N6-N7-N8-N9-N10-N11-
N12-N13-N14-N15-N16-N17-3′
(SEQ ID NO: 46)
5′-UAAUUUCUACUCUUGUAGAU-N1-N2-N3-N4-N5-N6-N7-N8-N9-N10-N11-
N12-N13-N14-N15-N16-N17-N18-3′
(SEQ ID NO: 47)
5′-UAAUUUCUACUCUUGUAGAU-N1-N2-N3-N4-N5-N6-N7-N8-N9-N10-N11-
N12-N13-N14-N15-N16-N17-N18-N19-3′
(SEQ ID NO: 48)
5′-UAAUUUCUACUCUUGUAGAU-N1-N2-N3-N4-N5-N6-N7-N8-N9-N10-N11-
N12-N13-N14-N15-N16-N17-N18-N19-N20-3′
(SEQ ID NO: 49)
5′-UAAUUUCUACUCUUGUAGAU-N1-N2-N3-N4-N5-N6-N7-N8-N9-N10-N11-
N12-N13-N14-N15-N16-N17-N18-N19-N20-N21-3′
(SEQ ID NO: 50)
5′-UAAUUUCUACUCUUGUAGAU-N1-N2-N3-N4-N5-N6-N7-N8-N9-N10-N11-
N12-N13-N14-N15-N16-N17-N18-N19-N20-N21-N22-3′
(SEQ ID NO: 51)
5′-UAAUUUCUACUCUUGUAGAU-N1-N2-N3-N4-N5-N6-N7-N8-N9-N10-N11-
N12-N13-N14-N15-N16-N17-N18-N19-N20-N21-N22-N23-3′
(SEQ ID NO: 52)
5′-UAAUUUCUACUCUUGUAGAU-N1-N2-N3-N4-N5-N6-N7-N8-N9-N10-N11-
N12-N13-N14-N15-N16-N17-N18-N19-N20-N21-N22-N23-N24-3′
(SEQ ID NO: 53)
5′-UAAUUUCUACUCUUGUAGAU-N1-N2-N3-N4-N5-N6-N7-N8-N9-N10-N11-
N12-N13-N14-N15-N16-N17-N18-N19-N20-N21-N22-N23-N24-N25-3′

In the exemplary Cas 12a gRNAs shown above, the position number of the nucleotides of the targeting domain are labeled relative to the 5′ end of the targeting domain.

As described herein, in some embodiments, the photocleavable moiety replaces a nucleotide at a position in the gRNA. The position numbering of the photocleavable moiety may be referred to as the nucleotide position number after which the moiety is located or the nucleotide position at which the photocleavable moiety is located. For example, a photocleavable moiety following position 1 (pos1) is present in the gRNA following the nucleotide at position 1, meaning between the first and third nucleotides of the gRNA, counting from the 5′ end (in place of the second nucleotide). In some embodiments, a photocleavable moiety is present at position N1, in place of a nucleotide at N1 between the last nucleotide of the crRNA sequence and N2 of the targeting domain. In some embodiments, a photocleavable moiety is present at position N2, in place of a nucleotide at N2 between N1 and N3. In some embodiments, a photocleavable moiety is present at position N3, in place of the nucleotide between N2 and N4. In some embodiments, a photocleavable moiety is present at position N4, in place of the nucleotide between N3 and N5. In some embodiments, a photocleavable moiety is present at position N5, in place of the nucleotide between N4 and N6. In some embodiments, a photocleavable moiety is present at position N6, in place of the nucleotide between N5 and N7. In some embodiments, a photocleavable moiety is present at position N7, in place of the nucleotide between N6 and N8. In some embodiments, a photocleavable moiety is present at position N8, in place of the nucleotide between N7 and N9. In some embodiments, a photocleavable moiety is present at position N9, in place of the nucleotide between N8 and N10. In some embodiments, a photocleavable moiety is present at position N10, in place of the nucleotide between N9 and N11. In some embodiments, a photocleavable moiety is present at position N11, in place of the nucleotide between N10 and N12. In some embodiments, a photocleavable moiety is present at position N12, in place of the nucleotide between N11 and N13. In some embodiments, a photocleavable moiety is present at position N13, in place of the nucleotide between N12 and N14. In some embodiments, a photocleavable moiety is present at position N14, in place of the nucleotide between N13 and N15. In some embodiments, a photocleavable moiety is present at position N15, in place of the nucleotide between N14 and N16. In some embodiments, a photocleavable moiety is present at position N16, in place of the nucleotide between N15 and N17. In some embodiments, a photocleavable moiety is present at position N17, in place of the nucleotide between N16 and N18. In some embodiments, a photocleavable moiety is present at position N18, in place of the nucleotide between N17 and N19. In some embodiments, a photocleavable moiety is present at position N19, in place of the nucleotide between N18 and N20. In some embodiments, a photocleavable I moiety is present at position N20, in place of the nucleotide between N19 and N21. In some embodiments, a photocleavable moiety is present at position N21, in place of the nucleotide between N20 and N22. In some embodiments, a photocleavable moiety is present at position N22, in place of the nucleotide between N21 and N23. In some embodiments, a photocleavable moiety is present at position N23, in place of the nucleotide between N22 and N24. In some embodiments, a photocleavable moiety is present at position N24. In some embodiments, a photocleavable moiety is present at position N25.

As described herein, in some embodiments, the photocleavable moiety is inserted between two nucleotides of the gRNA and therefore does not alter the nucleotide sequence of the gRNA. In some embodiments, the position numbering of the photocleavable moiety is the nucleotide position number after which the moiety is located. For example, a photocleavable moiety at position 1 (pos1) is present in the gRNA following the nucleotide at position 1, meaning between the first and second nucleotides of the gRNA, counting from the 5′ end. In some embodiments, a photocleavable moiety is present at position N1. In some embodiments, a photocleavable moiety is present at position N2. In some embodiments, a photocleavable moiety is present at position N3. In some embodiments, a photocleavable moiety is present at position N4. In some embodiments, a photocleavable moiety is present at position N5. In some embodiments, a photocleavable moiety is present at position N6. In some embodiments, a photocleavable moiety is present at position N7. In some embodiments, a photocleavable moiety is present at position N8. In some embodiments, a photocleavable moiety is present at position N9. In some embodiments, a photocleavable moiety is present at position N10. In some embodiments, a photocleavable moiety is present at position N11. In some embodiments, a photocleavable moiety is present at position N12. In some embodiments, a photocleavable moiety is present at position N13. In some embodiments, a photocleavable moiety is present at position N14. In some embodiments, a photocleavable moiety is present at position N15. In some embodiments, a photocleavable moiety is present at position N16. In some embodiments, a photocleavable moiety is present at position N17. In some embodiments, a photocleavable moiety is present at position N18. In some embodiments, a photocleavable moiety is present at position N19. In some embodiments, a photocleavable I moiety is present at position N20. In some embodiments, a photocleavable moiety is present at position N21. In some embodiments, a photocleavable moiety is present at position N22. In some embodiments, a photocleavable moiety is present at position N23. In some embodiments, a photocleavable moiety is present at position N24. In some embodiments, a photocleavable moiety is present at position N25.

In some embodiments, the photocleavable moiety comprises a nitroaryl group, e.g., an o-nitrobenzyl moiety or a derivative thereof. In some embodiments, an o-nitrobenzyl moiety comprises a benzene ring with a nitro group positioned ortho to a leaving group which may be in the benzylic position or bonded to the benzylic position. In some embodiments, the photocleavable group comprises a derivative of an o-nitrobenzyl moiety comprising one or more modifications to the o-nitrobenzyl moiety. For example, a modification to the aromatic group may adjust the absorbance of the o-nitrobenzyl moiety, or addition of a group at the benzylic position may adjust quantum yield of cleavage photo-products. In some embodiments, the o-nitrobenzyl moiety comprises an alkyl group (e.g., a methyl, ethyl, propyl, or butyl group) in the benzylic position, e.g., connected to an oligonucleotide (e.g., one or two nucleotides of the oligonucleotide). In some embodiments, the leaving group comprises an oligonucleotide, e.g., a gRNA. In some embodiments, the oligonucleotide, e.g., a gRNA or portion thereof, is attached to the o-nitrobenzyl moiety at the benzylic position. In some embodiments, radiation from 300-380, 300-360, 300-350, 300-340, 300-320, 320-380, 320-360, 320-350, 320-340, 340-380, 340-360, 340-350, 350-380, 350-360, or 360-380 nm induces photocleavage of an oligonucleotide comprising a photocleavable moiety comprising an o-nitrobenzyl moiety. In some embodiments, radiation of about 365 nm induces photocleavage of an oligonucleotide comprising a photocleavable moiety comprising an o-nitrobenzyl moiety.

In some embodiments, the photocleavable moiety comprises a suitable photocleavable group (or derivatives thereof) known in the art. Suitable photocleavable groups include, but are not limited to: nitroaryl groups, e.g., o-nitrobenzyl moieties or derivatives thereof, arylcarbonylmethyl groups, coumarin-4-ylmethyl groups, arylmethyl groups, metal-containing groups, pivaloyl groups, carboxylic acid esters, arylsulfonyl groups, sisyl/silicon comprising groups, 2-hydroxycinnamyl groups, α-Keto Amides, α,β-Unsaturated Anilides, and Methyl(phenyl)thiocarbamic Acid groups, thiochromone S,S-dioxide groups, 2-pyrrolidino-1,4-Benzoquinone groups, triazine/arylmethyleneimino groups, and xanthene/pyronin groups. See, e.g., Klán et al. Chem. Rev. (2013) 113, 119-191, which is hereby incorporated by reference in its entirety.

In some embodiments, a gRNA provided herein comprises a photocleavable moiety as described herein. In some embodiments, a gRNA provided herein comprises two or more photocleavable moieties. In some such embodiments, the two or more photocleavable moieties are of the same type, e.g., the two or more photocleavable moieties are nitroaryl groups. In other such embodiments, at least two of the two or more photocleavable moieties are of a different type.

As described herein, in some embodiments, the photocleavable moiety is inserted between two nucleotides of the gRNA and therefore does not alter the nucleotide sequence of the gRNA.

Table 1 provides an illustration of example photocleavable Cas12a gRNA designs (e.g., for use with an AsCpf1 nuclease). A photocleavable moiety as described herein is introduced into the gRNA at various positions, for example in the targeting domain. The crRNA sequence is shown in boldface with italics, the remaining sequence corresponds to the targeting domain. The photocleavable moiety is indicated in brackets as “pm.” The position numbering of the photocleavable moiety is the nucleotide position number after which the moiety is located. For example, a photocleavable moiety at position 1 (pos1) is present in the gRNA following the nucleotide at position 1, meaning between the first and second nucleotides of the gRNA.

TABLE 1

| Guide Name | gRNA Sequence (5′ to 3′) |
|---|---|
| Cpf1_pcgRNA_pos1 | 5′ U[pm]AAUUUCUACUCUUGUAGAUNNNNNNNNNNNNNNNNNNNNN 3′ |
| | (SEQ ID NO: 56) |
| Cpf1_pcgRNA_pos2 | 5′ UA[pm]AUUUCUACUCUUGUAGAUNNNNNNNNNNNNNNNNNNNNN 3′ |
| | (SEQ ID NO: 57) |
| Cpf1_pcgRNA_pos3 | 5′ UAA[pm]UUUCUACUCUUGUAGAUNNNNNNNNNNNNNNNNNNNNN3′ |
| | (SEQ ID NO: 57) |
| Cpf1_pcgRNA_pos4 | 5′ UAAU[pm]UUCUACUCUUGUAGAUNNNNNNNNNNNNNNNNNNNNN 3′ |
| | (SEQ ID NO: 58) |
| Cpf1_pcgRNA_pos5 | 5′ UAAUU[pm]UCUACUCUUGUAGAUNNNNNNNNNNNNNNNNNNNNN 3′ |
| | (SEQ ID NO: 59) |
| Cpf1_pcgRNA_pos6 | 5′ UAAUUU[pm]CUACUCUUGUAGAUNNNNNNNNNNNNNNNNNNNNN 3′ |
| | (SEQ ID NO: 60) |
| Cpf1_pcgRNA_pos7 | 5′ UAAUUUC[pm]UACUCUUGUAGAUNNNNNNNNNNNNNNNNNNNNN 3′ |
| | (SEQ ID NO: 61) |
| Cpf1_pcgRNA_pos8 | 5′ UAAUUUCU[pm]ACUCUUGUAGAUNNNNNNNNNNNNNNNNNNNNN 3′ |
| | (SEQ ID NO: 62) |
| Cpf1_pcgRNA_pos9 | 5′ UAAUUUCUA[pm]CUCUUGUAGAUNNNNNNNNNNNNNNNNNNNNN 3′ |
| | (SEQ ID NO: 63) |
| Cpf1_pcgRNA_pos10 | 5′ UAAUUUCUAC[pm]UCUUGUAGAUNNNNNNNNNNNNNNNNNNNNN 3′ |
| | (SEQ ID NO: 64) |
| Cpf1_pcgRNA_pos11 | 5′ UAAUUUCUACU[pm]CUUGUAGAUNNNNNNNNNNNNNNNNNNNNN 3′ |
| | (SEQ ID NO: 65) |
| Cpf1_pcgRNA_pos12 | 5′ UAAUUUCUACUC[pm]UUGUAGAUNNNNNNNNNNNNNNNNNNNNN 3′ |
| | (SEQ ID NO: 66) |
| Cpf1_pcgRNA_pos13 | 5′ UAAUUUCUACUCU[pm]UGUAGAUNNNNNNNNNNNNNNNNNNNNN 3′ |
| | (SEQ ID NO: 67) |
| Cpf1_pcgRNA_pos14 | 5′ UAAUUUCUACUCUU[pm]GUAGAUNNNNNNNNNNNNNNNNNNNNN 3′ |
| | (SEQ ID NO: 68) |
| Cpf1_pcgRNA_pos15 | 5′ UAAUUUCUACUCUUG[pm]UAGAUNNNNNNNNNNNNNNNNNNNNN 3′ |
| | (SEQ ID NO: 69) |
| Cpf1_pcgRNA_pos16 | 5′ UAAUUUCUACUCUUGU[pm]AGAUNNNNNNNNNNNNNNNNNNNNN 3′ |
| | (SEQ ID NO: 70) |
| Cpf1_pcgRNA_pos17 | 5′ UAAUUUCUACUCUUGUA[pm]GAUNNNNNNNNNNNNNNNNNNNNN 3′ |
| | (SEQ ID NO: 71) |
| Cpf1_pcgRNA_pos18 | 5′ UAAUUUCUACUCUUGUAG[pm]AUNNNNNNNNNNNNNNNNNNNNN 3′ |
| | (SEQ ID NO: 72) |
| Cpf1_pcgRNA_pos19 | 5′ UAAUUUCUACUCUUGUAGA[pm]UNNNNNNNNNNNNNNNNNNNNN 3′ |
| | (SEQ ID NO: 73) |
| Cpf1_pcgRNA_pos20 | 5′ UAAUUUCUACUCUUGUAGAU[pm]NNNNNNNNNNNNNNNNNNNNN 3′ |
| | (SEQ ID NO: 74) |
| Cpf1_pcgRNA_pos21 | 5′ UAAUUUCUACUCUUGUAGAUN[pm]NNNNNNNNNNNNNNNNNNNN 3′ |
| | (SEQ ID NO: 75) |
| Cpf1_pcgRNA_pos22 | 5′ UAAUUUCUACUCUUGUAGAUNN[pm]NNNNNNNNNNNNNNNNNNN 3′ |
| | (SEQ ID NO: 76) |
| Cpf1_pcgRNA_pos23 | 5′ UAAUUUCUACUCUUGUAGAUNNN[pm]NNNNNNNNNNNNNNNNNN 3′ |
| | (SEQ ID NO: 77) |
| Cpf1_pcgRNA_pos24 | 5′ UAAUUUCUACUCUUGUAGAUNNNN[pm]NNNNNNNNNNNNNNNNN 3′ |
| | (SEQ ID NO: 78) |
| Cpf1_pcgRNA_pos25 | 5′ UAAUUUCUACUCUUGUAGAUNNNNN[pm]NNNNNNNNNNNNNNNN 3′ |
| | (SEQ ID NO: 79) |
| Cpf1_pcgRNA_pos26 | 5′ UAAUUUCUACUCUUGUAGAUNNNNNN[pm]NNNNNNNNNNNNNNN 3′ |
| | (SEQ ID NO: 80) |
| Cpf1_pcgRNA_pos27 | 5′ UAAUUUCUACUCUUGUAGAUNNNNNNN[pm]NNNNNNNNNNNNNN 3′ |
| | (SEQ ID NO: 81) |
| Cpf1_pcgRNA_pos28 | 5′ UAAUUUCUACUCUUGUAGAUNNNNNNNN[pm]NNNNNNNNNNNNN 3′ |
| | (SEQ ID NO: 82) |
| Cpf1_pcgRNA_pos29 | 5′ UAAUUUCUACUCUUGUAGAUNNNNNNNNN[pm]NNNNNNNNNNNN 3′ |
| | (SEQ ID NO: 83) |
| Cpf1_pcgRNA_pos30 | 5′ UAAUUUCUACUCUUGUAGAUNNNNNNNNNN[pm]NNNNNNNNNNN 3′ |
| | (SEQ ID NO: 84) |
| Cpf1_pcgRNA_pos31 | 5′ UAAUUUCUACUCUUGUAGAUNNNNNNNNNNN[pm]NNNNNNNNNN 3′ |
| | (SEQ ID NO: 85) |
| Cpf1_pcgRNA_pos32 | 5′ UAAUUUCUACUCUUGUAGAUNNNNNNNNNNNN[pm]NNNNNNNNN 3′ |
| | (SEQ ID NO: 86) |
| Cpf1_pcgRNA_pos33 | 5′ UAAUUUCUACUCUUGUAGAUNNNNNNNNNNNNN[pm]NNNNNNNN 3′ |
| | (SEQ ID NO: 87) |
| Cpf1_pcgRNA_pos34 | 5′ UAAUUUCUACUCUUGUAGAUNNNNNNNNNNNNNN[pm]NNNNNNN 3′ |
| | (SEQ ID NO: 88) |
| Cpf1_pcgRNA_pos35 | 5′ UAAUUUCUACUCUUGUAGAUNNNNNNNNNNNNNNN[pm]NNNNNN 3′ |
| | (SEQ ID NO: 89) |
| Cpf1_pcgRNA_pos36 | 5′ UAAUUUCUACUCUUGUAGAUNNNNNNNNNNNNNNNN[pm]NNNNN 3′ |
| | (SEQ ID NO: 90) |
| Cpf1_pcgRNA_pos37 | 5′ UAAUUUCUACUCUUGUAGAUNNNNNNNNNNNNNNNNN[pm]NNNN 3′ |
| | (SEQ ID NO: 91) |
| Cpf1_pcgRNA_pos38 | 5′ UAAUUUCUACUCUUGUAGAUNNNNNNNNNNNNNNNNNN[pm]NNN 3′ |
| | (SEQ ID NO: 92) |
| Cpf1_pcgRNA_pos39 | 5′ UAAUUUCUACUCUUGUAGAUNNNNNNNNNNNNNNNNNNN[pm]NN 3′ |
| | (SEQ ID NO: 93) |
| Cpf1_pcgRNA_pos40 | 5′ UAAUUUCUACUCUUGUAGAUNNNNNNNNNNNNNNNNNNNN[pm]N 3′ |
| | (SEQ ID NO: 94) |

Table 2 provides an illustration of example photocleavable gRNA designs targeting CD19 using a Cas12a nuclease (e.g., AsCpf1). As demonstrated, a photocleavable moiety. (e.g., 2-nitrobenzyl bond) is introduced into the gRNA at various positions, for example in the targeting domain. The crRNA sequence is shown in boldface with italics, the remaining sequence corresponds to the targeting region. The exemplary photocleavable moiety illustrated here (a 2-nitrobenzyl bond) is indicated in brackets as “nb.” Embodiments in which the “nb” moiety is replaced by any other suitable photocleavable moiety are also embraced by this disclosure. The position numbering of the photocleavable moiety is the nucleotide position number after which the moiety is located. For example, a photocleavable moiety at position 1 (pos1) is present in the gRNA following the nucleotide at position 1, meaning between the first and second nucleotides of the gRNA.

TABLE 2

| Guide Name | gRNA Sequence (5′ to 3′) |
|---|---|
| CD19_pos1 | 5′ U[nb]AAUUUCUACUCUUGUAGAUAGCGGGGACUCCCGAGACCAG 3′ |
| | (SEQ ID NO: 1) |
| CD19_pos2 | 5′ UA[nb]AUUUCUACUCUUGUAGAUAGCGGGGACUCCCGAGACCAG3′ |
| | (SEQ ID NO: 2) |
| CD19_pos3 | 5′ UAA[nb]UUUCUACUCUUGUAGAUAGCGGGGACUCCCGAGACCAG3′ |
| | (SEQ ID NO: 3) |
| CD19_pos4 | 5′ UAAU[nb]UUCUACUCUUGUAGAUAGCGGGGACUCCCGAGACCAG 3′ |
| | (SEQ ID NO: 4) |
| CD19_pos5 | 5′ UAAUU[nb]UCUACUCUUGUAGAUAGCGGGGACUCCCGAGACCAG 3′ |
| | (SEQ ID NO: 5) |
| CD19_pos6 | 5′ UAAUUU[nb]CUACUCUUGUAGAUAGCGGGGACUCCCGAGACCAG 3′ |
| | (SEQ ID NO: 6) |
| CD19_pos7 | 5′ UAAUUUC[nb]UACUCUUGUAGAUAGCGGGGACUCCCGAGACCAG 3′ |
| | (SEQ ID NO: 7) |
| CD19_pos8 | 5′ UAAUUUCU[nb]ACUCUUGUAGAUAGCGGGGACUCCCGAGACCAG 3′ |
| | (SEQ ID NO: 8) |
| CD19_pos9 | 5′ UAAUUUCUA[nb]CUCUUGUAGAUAGCGGGGACUCCCGAGACCAG 3′ |
| | (SEQ ID NO: 9) |
| CD19_pos10 | 5′ UAAUUUCUAC[nb]UCUUGUAGAUAGCGGGGACUCCCGAGACCAG 3′ |
| | (SEQ ID NO: 10) |
| CD19_pos11 | 5′ UAAUUUCUACU[nb]CUUGUAGAUAGCGGGGACUCCCGAGACCAG 3′ |
| | (SEQ ID NO: 11) |
| CD19_pos12 | 5′ UAAUUUCUACUC[nb]UUGUAGAUAGCGGGGACUCCCGAGACCAG 3′ |
| | (SEQ ID NO: 12) |
| CD19_pos13 | 5′ UAAUUUCUACUCU[nb]UGUAGAUAGCGGGGACUCCCGAGACCAG 3′ |
| | (SEQ ID NO: 13) |
| CD19_pos14 | 5′ UAAUUUCUACUCUU[nb]GUAGAUAGCGGGGACUCCCGAGACCAG 3′ |
| | (SEQ ID NO: 14) |
| CD19_pos15 | 5′ UAAUUUCUACUCUUG[nb]UAGAUAGCGGGGACUCCCGAGACCAG 3′ |
| | (SEQ ID NO: 15) |
| CD19_pos16 | 5′ UAAUUUCUACUCUUGU[nb]AGAUAGCGGGGACUCCCGAGACCAG 3′ |
| | (SEQ ID NO: 16) |
| CD19_pos17 | 5′ UAAUUUCUACUCUUGUA[nb]GAUAGCGGGGACUCCCGAGACCAG 3′ |
| | (SEQ ID NO: 17) |
| CD19_pos18 | 5′ UAAUUUCUACUCUUGUAG[nb]AUAGCGGGGACUCCCGAGACCAG 3′ |
| | (SEQ ID NO: 18) |
| CD19_pos19 | 5′ UAAUUUCUACUCUUGUAGA[nb]UAGCGGGGACUCCCGAGACCAG 3′ |
| | (SEQ ID NO: 19) |
| CD19_pos20 | 5′ UAAUUUCUACUCUUGUAGAU[nb]AGCGGGGACUCCCGAGACCAG 3′ |
| | (SEQ ID NO: 20) |
| CD19_pos21 | 5′ UAAUUUCUACUCUUGUAGAUA[nb]GCGGGGACUCCCGAGACCAG 3′ |
| | (SEQ ID NO: 21) |
| CD19_pos22 | 5′ UAAUUUCUACUCUUGUAGAUAG[nb]CGGGGACUCCCGAGACCAG 3′ |
| | (SEQ ID NO: 22) |
| CD19_pos23 | 5′ UAAUUUCUACUCUUGUAGAUAGC[nb]GGGGACUCCCGAGACCAG 3′ |
| | (SEQ ID NO: 23) |
| CD19_pos24 | 5′ UAAUUUCUACUCUUGUAGAUAGCG[nb]GGGACUCCCGAGACCAG 3′ |
| | (SEQ ID NO: 24) |
| CD19_pos25 | 5′ UAAUUUCUACUCUUGUAGAUAGCGG[nb]GGACUCCCGAGACCAG 3′ |
| | (SEQ ID NO: 25) |
| CD19_pos26 | 5′ UAAUUUCUACUCUUGUAGAUAGCGGG[nb]GACUCCCGAGACCAG 3′ |
| | (SEQ ID NO: 26) |
| CD19_pos27 | 5′ UAAUUUCUACUCUUGUAGAUAGCGGGG[nb]ACUCCCGAGACCAG 3′ |
| | (SEQ ID NO: 27) |
| CD19_pos28 | 5′ UAAUUUCUACUCUUGUAGAUAGCGGGGA[b]CUCCCGAGACCAG 3′ |
| | (SEQ ID NO: 28) |
| CD19_pos29 | 5′ UAAUUUCUACUCUUGUAGAUAGCGGGGAC[nb]UCCCGAGACCAG 3′ |
| | (SEQ ID NO: 29) |
| CD19_pos30 | 5′ UAAUUUCUACUCUUGUAGAUAGCGGGGACU[nb]CCCGAGACCAG 3′ |
| | (SEQ ID NO: 30) |
| CD19_pos31 | 5′ UAAUUUCUACUCUUGUAGAUAGCGGGGACUC[nb]CCGAGACCAG 3′ |
| | (SEQ ID NO: 31) |
| CD19_pos32 | 5′ UAAUUUCUACUCUUGUAGAUAGCGGGGACUCC[nb]CGAGACCAG 3′ |
| | (SEQ ID NO: 32) |
| CD19_pos33 | 5′ UAAUUUCUACUCUUGUAGAUAGCGGGGACUCCC[nb]GAGACCAG 3′ |
| | (SEQ ID NO: 33) |
| CD19_pos34 | 5′ UAAUUUCUACUCUUGUAGAUAGCGGGGACUCCCG[nb]AGACCAG 3′ |
| | (SEQ ID NO: 34) |
| CD19_pos35 | 5′ UAAUUUCUACUCUUGUAGAUAGCGGGGACUCCCGA[nb]GACCAG 3′ |
| | (SEQ ID NO: 35) |
| CD19_pos36 | 5′ UAAUUUCUACUCUUGUAGAUAGCGGGGACUCCCGAG[nb]ACCAG 3′ |
| | (SEQ ID NO: 36) |
| CD19_pos37 | 5′ UAAUUUCUACUCUUGUAGAUAGCGGGGACUCCCGAGA[nb]CCAG 3′ |
| | (SEQ ID NO: 37) |
| CD19_pos38 | 5′ UAAUUUCUACUCUUGUAGAUAGCGGGGACUCCCGAGAC[nb]CAG 3′ |
| | (SEQ ID NO: 38) |
| CD19_pos39 | 5′ UAAUUUCUACUCUUGUAGAUAGCGGGGACUCCCGAGACC[nb]AG 3′ |
| | (SEQ ID NO: 39) |
| CD19_pos40 | 5′ UAAUUUCUACUCUUGUAGAUAGCGGGGACUCCCGAGACCA[nb]G 3′ |
| | (SEQ ID NO: 40) |

Table 3 provides an illustration of example photocleavable gRNA designs targeting CD5, VEGF, or CD33 using a Cas9 nuclease (e.g., SpCas9). As demonstrated, a photocleavable moiety (e.g., 2-nitrobenzyl bond) is introduced into the gRNA at various positions, for example within the first 15 nucleotides from the 5′ end of the gRNA. The exemplary photocleavable moiety illustrated here (a 2-nitrobenzyl bond) is indicated by “nb” in boldface with underline. Embodiments in which the “nb” moiety is replaced by any other suitable photocleavable moiety are also embraced by this disclosure.

TABLE 3

| Guide Name | gRNA Sequence (5′ to 3′) |
|---|---|
| CD5_guide | 5′csasusAGnbCUGAUGGUACCCCCCGUUUUAGAGCUAGAAAUAG |
| | CAAGUUAAAAUAAGGCUAGUCCGUUAUCAACUUGAAAAAGUGGCACC |
| | GAGUCGGUGCusususU3′ |
| | (SEQ ID NO: 41) |
| VEGFA_guide | 5′gsascsCCnbCCUCCACCCCGCCUCGUUUUAGAGCUAGAAAUAGC |
| | AAGUUAAAAUAAGGCUAGUCCGUUAUCAACUUGAAAAAGUGGCACCG |
| | AGUCGGUGCusususU3′ |
| | (SEQ ID NO: 42) |
| CD33_guide | 5′gsgsusGGnbGGGCAGCUGACAACCGUUUUAGAGCUAGAAAUAGCA |
| | AGUUAAAAUAAGGCUAGUCCGUUAUCAACUUGAAAAAGUGGCACCGAG |
| | UCGGUGCusususU3′ |
| | (SEQ ID NO: 43) |

In the tables above, lowercase nucleotides (c, a, u, g) correspond to 2′-O-methyl nucleotides and capitalized nucleotides (C, A, U, G) correspond to RNA nucleotides. The lowercase “s” refers to a phosphorothioate.

As described herein, in some embodiments, the photocleavable moiety replaces a nucleotide of the gRNA, such as a nucleotide of the targeting domain corresponding to the target sequence, or a nucleotide of the crRNA domain. In some embodiments, the photocleavable moiety replaces a nucleotide of the gRNA, such as a nucleotide of the targeting domain corresponding to the target sequence, or a nucleotide of the crRNA domain as compared to a gRNA that targets the target site sequence and does not include a photocleavable moiety.

Table 4 provides an illustration of example photocleavable Cas12a gRNA designs (e.g., for use with an AsCpf1 nuclease). A photocleavable moiety as described herein is introduced into the gRNA at various positions, for example in the targeting domain, replacing a nucleotide corresponding to the target sequence. The crRNA sequence is shown in boldface with italics, the remaining sequence corresponds to the targeting domain. The photocleavable moiety is indicated in brackets as “pm.” In Table 4, the “Guide Name” refers to the position number of the photocleavable moiety is the nucleotide position number after which the moiety is located. For example, a photocleavable moiety at position 1 (pos1) is present in the gRNA following the nucleotide at position 1, meaning between the first and third nucleotides of the gRNA, in place of the second nucleotide.

TABLE 4

| Guide Name | gRNA Sequence (5′ to 3′) |
|---|---|
| Cpf1_pcgRNA_pos1 | 5′ U[pm]AUUUCUACUCUUGUAGAUNNNNNNNNNNNNNNNNNNNNN 3′ |
| | (SEQ ID NO: 95) |
| Cpf1_pcgRNA_pos2 | 5′ UA[pm]UUUCUACUCUUGUAGAUNNNNNNNNNNNNNNNNNNNNN 3′ |
| | (SEQ ID NO: 96) |
| Cpf1_pcgRNA_pos3 | 5′ UAA[pm]UUCUACUCUUGUAGAUNNNNNNNNNNNNNNNNNNNNN 3′ |
| | (SEQ ID NO: 97) |
| Cpf1_pcgRNA_pos4 | 5′ UAAU[pm]UCUACUCUUGUAGAUNNNNNNNNNNNNNNNNNNNNN 3′ |
| | (SEQ ID NO: 98) |
| Cpf1_pcgRNA_pos5 | 5′ UAAUU[pm]CUACUCUUGUAGAUNNNNNNNNNNNNNNNNNNNNN 3′ |
| | (SEQ ID NO: 99) |
| Cpf1_pcgRNA_pos6 | 5′ UAAUUU[pm]UACUCUUGUAGAUNNNNNNNNNNNNNNNNNNNNN 3′ |
| | (SEQ ID NO: 100) |
| Cpf1_pcgRNA_pos7 | 5′ UAAUUUC[pm]ACUCUUGUAGAUNNNNNNNNNNNNNNNNNNNNN 3′ |
| | (SEQ ID NO: 101) |
| Cpf1_pcgRNA_pos8 | 5′ UAAUUUCU[pm]CUCUUGUAGAUNNNNNNNNNNNNNNNNNNNNN 3′ |
| | (SEQ ID NO: 102) |
| Cpf1_pcgRNA_pos9 | 5′ UAAUUUCUA[pm]UCUUGUAGAUNNNNNNNNNNNNNNNNNNNNN 3′ |
| | (SEQ ID NO: 103) |
| Cpf1_pcgRNA_pos10 | 5′ UAAUUUCUAC[pm]CUUGUAGAUNNNNNNNNNNNNNNNNNNNNN 3′ |
| | (SEQ ID NO: 104) |
| Cpf1_pcgRNA_pos11 | 5′ UAAUUUCUACU[pm]UUGUAGAUNNNNNNNNNNNNNNNNNNNNN 3′ |
| | (SEQ ID NO: 105) |
| Cpf1_pcgRNA_pos12 | 5′ UAAUUUCUACUC[pm]UGUAGAUNNNNNNNNNNNNNNNNNNNNN 3′ |
| | (SEQ ID NO: 106) |
| Cpf1_pcgRNA_pos13 | 5′ UAAUUUCUACUCU[pm]GUAGAUNNNNNNNNNNNNNNNNNNNNN 3′ |
| | (SEQ ID NO: 107) |
| Cpf1_pcgRNA_pos14 | 5′ UAAUUUCUACUCUU[pm]UAGAUNNNNNNNNNNNNNNNNNNNNN 3′ |
| | (SEQ ID NO: 108) |
| Cpf1_pcgRNA_pos15 | 5′ UAAUUUCUACUCUUG[pm]AGAUNNNNNNNNNNNNNNNNNNNNN 3′ |
| | (SEQ ID NO: 109) |
| Cpf1_pcgRNA_pos16 | 5′ UAAUUUCUACUCUUGU[pm]GAUNNNNNNNNNNNNNNNNNNNNN 3′ |
| | (SEQ ID NO: 110) |
| Cpf1_pcgRNA_pos17 | 5′ UAAUUUCUACUCUUGUA[pm]AUNNNNNNNNNNNNNNNNNNNNN 3′ |
| | (SEQ ID NO: 111) |
| Cpf1_pcgRNA_pos18 | 5′ UAAUUUCUACUCUUGUAG[pm]UNNNNNNNNNNNNNNNNNNNNN 3′ |
| | (SEQ ID NO: 112) |
| Cpf1_pcgRNA_pos19 | 5′ UAAUUUCUACUCUUGUAGA[pm]NNNNNNNNNNNNNNNNNNNNN 3′ |
| | (SEQ ID NO: 113) |
| Cpf1_pcgRNA_pos20 | 5′ UAAUUUCUACUCUUGUAGAU[pm]NNNNNNNNNNNNNNNNNNNN 3′ |
| | (SEQ ID NO: 114) |
| Cpf1_pcgRNA_pos21 | 5′ UAAUUUCUACUCUUGUAGAUN[pm]NNNNNNNNNNNNNNNNNNN 3′ |
| | (SEQ ID NO: 115) |
| Cpf1_pcgRNA_pos22 | 5′ UAAUUUCUACUCUUGUAGAUNN[pm]NNNNNNNNNNNNNNNNNN 3′ |
| | (SEQ ID NO: 116) |
| Cpf1_pcgRNA_pos23 | 5′ UAAUUUCUACUCUUGUAGAUNNN[pm]NNNNNNNNNNNNNNNNN 3′ |
| | (SEQ ID NO: 117) |
| Cpf1_pcgRNA_pos24 | 5′ UAAUUUCUACUCUUGUAGAUNNNN[pm]NNNNNNNNNNNNNNNN 3′ |
| | (SEQ ID NO: 118) |
| Cpf1_pcgRNA_pos25 | 5′ UAAUUUCUACUCUUGUAGAUNNNNN[pm]NNNNNNNNNNNNNNN 3′ |
| | (SEQ ID NO: 119) |
| Cpf1_pcgRNA_pos26 | 5′ UAAUUUCUACUCUUGUAGAUNNNNNN[pm]NNNNNNNNNNNNNN 3′ |
| | (SEQ ID NO: 120) |
| Cpf1_pcgRNA_pos27 | 5′ UAAUUUCUACUCUUGUAGAUNNNNNNN[pm]NNNNNNNNNNNNN 3′ |
| | (SEQ ID NO: 121) |
| Cpf1_pcgRNA_pos28 | 5′ UAAUUUCUACUCUUGUAGAUNNNNNNNN[pm]NNNNNNNNNNNN 3′ |
| | (SEQ ID NO: 122) |
| Cpf1_pcgRNA_pos29 | 5′ UAAUUUCUACUCUUGUAGAUNNNNNNNNN[pm]NNNNNNNNNNN 3′ |
| | (SEQ ID NO: 123) |
| Cpf1_pcgRNA_pos30 | 5′ UAAUUUCUACUCUUGUAGAUNNNNNNNNNN[pm]NNNNNNNNNN 3′ |
| | (SEQ ID NO: 124) |
| Cpf1_pcgRNA_pos31 | 5′ UAAUUUCUACUCUUGUAGAUNNNNNNNNNNN[pm]NNNNNNNNN 3′ |
| | (SEQ ID NO: 125) |
| Cpf1_pcgRNA_pos32 | 5′ UAAUUUCUACUCUUGUAGAUNNNNNNNNNNNN[pm]NNNNNNNN 3′ |
| | (SEQ ID NO: 126) |
| Cpf1_pcgRNA_pos33 | 5′ UAAUUUCUACUCUUGUAGAUNNNNNNNNNNNNN[pm]NNNNNNN 3′ |
| | (SEQ ID NO: 127) |
| Cpf1_pcgRNA_pos34 | 5′ UAAUUUCUACUCUUGUAGAUNNNNNNNNNNNNNN[pm]NNNNNN 3′ |
| | (SEQ ID NO: 128) |
| Cpf1_pcgRNA_pos35 | 5′ UAAUUUCUACUCUUGUAGAUNNNNNNNNNNNNNNN[pm]NNNNN 3′ |
| | (SEQ ID NO: 129) |
| Cpf1_pcgRNA_pos36 | 5′ UAAUUUCUACUCUUGUAGAUNNNNNNNNNNNNNNNN[pm]NNNN 3′ |
| | (SEQ ID NO: 130) |
| Cpf1_pcgRNA_pos37 | 5′ UAAUUUCUACUCUUGUAGAUNNNNNNNNNNNNNNNNN[pm]NNN 3′ |
| | (SEQ ID NO: 131) |
| Cpf1_pcgRNA_pos38 | 5′ UAAUUUCUACUCUUGUAGAUNNNNNNNNNNNNNNNNNN[pm]NN 3′ |
| | (SEQ ID NO: 132) |
| Cpf1_pcgRNA_pos39 | 5′ UAAUUUCUACUCUUGUAGAUNNNNNNNNNNNNNNNNNNN[pm]N 3′ |
| | (SEQ ID NO: 133) |

Table 5 provides an illustration of example photocleavable gRNA designs targeting CD19 using a Cas12a nuclease (e.g., AsCpf1). As demonstrated, a photocleavable moiety (e.g., 2-nitrobenzyl bond) is introduced into the gRNA at various positions, for example in the targeting domain, replacing a nucleotide corresponding to the target sequence. The crRNA sequence is shown in boldface with italics, the remaining sequence corresponds to the targeting region. The exemplary photocleavable moiety illustrated here (a 2-nitrobenzyl bond) is indicated in brackets as “nb.” Embodiments in which the “nb” moiety is replaced by any other suitable photocleavable moiety are also embraced by this disclosure. In Table 5, the “Guide Name” refers to the position number of the photocleavable moiety as the nucleotide position number after which the moiety is located. For example, a photocleavable moiety after position 1 (pos1) is present in the gRNA following the nucleotide at position 1, meaning between the first and third nucleotide of the gRNA, in place of the second nucleotide.

TABLE 5

| Guide Name | gRNA Sequence (5′ to 3′) |
|---|---|
| CD19_pos1 | 5′ U[nb]AUUUCUACUCUUGUAGAUAGCGGGGACUCCCGAGACCAG3′ |
| | (SEQ ID NO: 134) |
| CD19_pos2 | 5′ UA[nb]UUUCUACUCUUGUAGAUAGCGGGGACUCCCGAGACCAG3′ |
| | (SEQ ID NO: 135) |
| CD19_pos3 | 5′ UAA[nb]UUCUACUCUUGUAGAUAGCGGGGACUCCCGAGACCAG 3′ |
| | (SEQ ID NO: 136) |
| CD19_pos4 | 5′ UAAU[nb]UCUACUCUUGUAGAUAGCGGGGACUCCCGAGACCAG 3′ |
| | (SEQ ID NO: 137) |
| CD19_pos5 | 5′ UAAUU[nb]CUACUCUUGUAGAUAGCGGGGACUCCCGAGACCAG 3′ |
| | (SEQ ID NO: 138) |
| CD19_pos6 | 5′ UAAUUU[nb]UACUCUUGUAGAUAGCGGGGACUCCCGAGACCAG 3′ |
| | (SEQ ID NO: 139) |
| CD19_pos7 | 5′ UAAUUUC[nb]ACUCUUGUAGAUAGCGGGGACUCCCGAGACCAG 3′ |
| | (SEQ ID NO: 140) |
| CD19_pos8 | 5′ UAAUUUCU[nb]CUCUUGUAGAUAGCGGGGACUCCCGAGACCAG 3′ |
| | (SEQ ID NO: 141) |
| CD19_pos9 | 5′ UAAUUUCUA[nb]UCUUGUAGAUAGCGGGGACUCCCGAGACCAG 3′ |
| | (SEQ ID NO: 142) |
| CD19_pos10 | 5′ UAAUUUCUAC[nb]CUUGUAGAUAGCGGGGACUCCCGAGACCAG 3′ |
| | (SEQ ID NO: 143) |
| CD19_pos11 | 5′ UAAUUUCUACU[nb]UUGUAGAUAGCGGGGACUCCCGAGACCAG 3′ |
| | (SEQ ID NO: 144) |
| CD19_pos12 | 5′ UAAUUUCUACUC[nb]UGUAGAUAGCGGGGACUCCCGAGACCAG 3′ |
| | (SEQ ID NO: 145) |
| CD19_pos13 | 5′ UAAUUUCUACUCU[nb]GUAGAUAGCGGGGACUCCCGAGACCAG 3′ |
| | (SEQ ID NO: 146) |
| CD19_pos14 | 5′ UAAUUUCUACUCUU[nb]UAGAUAGCGGGGACUCCCGAGACCAG 3′ |
| | (SEQ ID NO: 147) |
| CD19_pos15 | 5′ UAAUUUCUACUCUUG[nb]AGAUAGCGGGGACUCCCGAGACCAG 3′ |
| | (SEQ ID NO: 148) |
| CD19_pos16 | 5′ UAAUUUCUACUCUUGU[nb]GAUAGCGGGGACUCCCGAGACCAG 3′ |
| | (SEQ ID NO: 149) |
| CD19_pos17 | 5′ UAAUUUCUACUCUUGUA[nb]AUAGCGGGGACUCCCGAGACCAG 3′ |
| | (SEQ ID NO: 150) |
| CD19_pos18 | 5′ UAAUUUCUACUCUUGUAG[nb]UAGCGGGGACUCCCGAGACCAG 3′ |
| | (SEQ ID NO: 151) |
| CD19_pos19 | 5′ |
| | UAAUUUCUACUCUUGUAGA[nb]AGCGGGGACUCCCGAGACCAG 3′ |
| | (SEQ ID NO: 152) |
| CD19_pos20 | 5′ |
| | UAAUUUCUACUCUUGUAGAU[nb]GCGGGGACUCCCGAGACCAG 3′ |
| | (SEQ ID NO: 153) |
| CD19_pos21 | 5′ UAAUUUCUACUCUUGUAGAUA[nb]CGGGGACUCCCGAGACCAG 3′ |
| | (SEQ ID NO: 154) |
| CD19_pos22 | 5′ UAAUUUCUACUCUUGUAGAUAG[nb]GGGGACUCCCGAGACCAG 3′ |
| | (SEQ ID NO: 155) |
| CD19_pos23 | 5′ |
| | UAAUUUCUACUCUUGUAGAUAGC[nb]GGGACUCCCGAGACCAG 3′ |
| | (SEQ ID NO: 156) |
| CD19_pos24 | 5′ UAAUUUCUACUCUUGUAGAUAGCG[nb]GGACUCCCGAGACCAG 3′ |
| | (SEQ ID NO: 157) |
| CD19_pos25 | 5′ |
| | UAAUUUCUACUCUUGUAGAUAGCGG[nb]GACUCCCGAGACCAG 3′ |
| | (SEQ ID NO: 158) |
| CD19_pos26 | 5′ UAAUUUCUACUCUUGUAGAUAGCGGG[nb]ACUCCCGAGACCAG 3′ |
| | (SEQ ID NO: 159) |
| CD19_pos27 | 5′ UAAUUUCUACUCUUGUAGAUAGCGGGG[nb]CUCCCGAGACCAG 3′ |
| | (SEQ ID NO: 160) |
| CD19_pos28 | 5′ UAAUUUCUACUCUUGUAGAUAGCGGGGA[nb]UCCCGAGACCAG 3′ |
| | (SEQ ID NO: 161) |
| CD19_pos29 | 5′ UAAUUUCUACUCUUGUAGAUAGCGGGGAC[nb]CCCGAGACCAG 3′ |
| | (SEQ ID NO: 162) |
| CD19_pos30 | 5′ UAAUUUCUACUCUUGUAGAUAGCGGGGACU[nb]CCGAGACCAG 3′ |
| | (SEQ ID NO: 163) |
| CD19_pos31 | 5′ UAAUUUCUACUCUUGUAGAUAGCGGGGACUC[nb]CGAGACCAG 3′ |
| | (SEQ ID NO: 164) |
| CD19_pos32 | 5′ UAAUUUCUACUCUUGUAGAUAGCGGGGACUCC[nb]GAGACCAG 3′ |
| | (SEQ ID NO: 165) |
| CD19_pos33 | 5′ UAAUUUCUACUCUUGUAGAUAGCGGGGACUCCC[nb]AGACCAG 3′ |
| | (SEQ ID NO: 166) |
| CD19_pos34 | 5′ UAAUUUCUACUCUUGUAGAUAGCGGGGACUCCCG[nb]GACCAG 3′ |
| | (SEQ ID NO: 167) |
| CD19_pos35 | 5′ |
| | UAAUUUCUACUCUUGUAGAUAGCGGGGACUCCCGA[nb]ACCAG 3′ |
| | (SEQ ID NO: 168) |
| CD19_pos36 | 5′ UAAUUUCUACUCUUGUAGAUAGCGGGGACUCCCGAG[nb]CCAG 3′ |
| | (SEQ ID NO: 169) |
| CD19_pos37 | 5′ |
| | UAAUUUCUACUCUUGUAGAUAGCGGGGACUCCCGAGA[nb]CAG 3′ |
| | (SEQ ID NO: 170) |
| CD19_pos38 | 5′ UAAUUUCUACUCUUGUAGAUAGCGGGGACUCCCGAGAC[nb]AG 3′ |
| | (SEQ ID NO: 171) |
| CD19_pos39 | 5′ UAAUUUCUACUCUUGUAGAUAGCGGGGACUCCCGAGACC[nb]G 3′ |
| | (SEQ ID NO: 172) |

Table 6 provides an illustration of example photocleavable targeting domain designs for gRNAs targeting CD5, VEGF, CD33, or BCL11A using a Cas9 nuclease (e.g., SpCas9). As demonstrated, a photocleavable moiety (e.g., 2-nitrobenzyl bond) is introduced into the targeting domain sequence of the original guide at position 6, meaning between the fifth and seventh nucleotides, in place of the sixth nucleotide of the original sequence.

TABLE 6

| Guide Name | Targeting Domain Sequence (5′ to 3′) |
|---|---|
| CD5_guide (original) | 5′-CAUAGCUGAUGGUACCCCCC-3′ |
| | (SEQ ID NO: 173) |
| CD5_guide (pm at | 5′-CAUAG[pm]UGAUGGUACCCCCC-3′ |
| position 6) | (SEQ ID NO: 174) |
| VEGFA_guide | 5′-GACCCCCUCCACCCCGCCUC-3′ |
| (original) | (SEQ ID NO: 175) |
| VEGFA_guide (pm | 5′-GACCC[pm]CUCCACCCCGCCUC-3′ |
| at position 6) | (SEQ ID NO: 176) |
| CD33_guide | 5′-GGUGGGGGCAGCUGACAACC-3′ |
| (original) | (SEQ ID NO: 177) |
| CD33_guide (pm at | 5′-GGUGG[pm]GGCAGCUGACAACC-3′ |
| position 6) | (SEQ ID NO: 178) |
| CBE_BCL11A_guide | 5′-UUUAUCACAGGCUCCAGGAA-3′ |
| (original) | (SEQ ID NO: 179) |
| CBE_BCL11A_guide | 5′-UUUAU[pm]ACAGGCUCCAGGAA-3′ |
| (pm at position 6) | (SEQ ID NO: 180) |

Table 7 provides an illustration of example photocleavable gRNA designs targeting CD5, VEGF, CD33, or BCL11A using a Cas9 nuclease (e.g., SpCas9). As demonstrated, a photocleavable moiety (e.g., 2-nitrobenzyl bond) is introduced into the targeting domain sequence at position 6, meaning between the fifth and seventh nucleotides, in place of the sixth nucleotide of the original sequence. In Table 7, the “Guide Name” for each gRNA includes “PC5,” which refers insertion of the photocleavable moiety following the nucleotide at position 5.

TABLE 7

| Guide Name | gRNA Sequence (5′ to 3′) |
|---|---|
| CD5_PC5 | 5′- |
| | mC*mA*mU*rArG/iSpPC/rUrGrArUrGrGrUrArCrCrCrCrCrCr |
| | GrUrUrUrUrArGrArGrCrUrArGrArArArUrArGrCrArArGrUrU |
| | rArArArArUrArArGrGrCrUrArGrUrCrCrGrUrUrArUrCrArAr |
| | CrUrUrGrArArArArArGrUrGrGrCrArCrCrGrArGrUrCrGrGrU |
| | rGrCmU*mU*mU*rU-3′ |
| | (SEQ ID NO: 181) |
| VEGFA_site2_PC5 | 5′- |
| | mG*mA*mC*rCrC/iSpPC/rCrUrCrCrArCrCrCrCrGrCrCrUrCr |
| | GrUrUrUrUrArGrArGrCrUrArGrArArArUrArGrCrArArGrUrU |
| | rArArArArUrArArGrGrCrUrArGrUrCrCrGrUrUrArUrCrArAr |
| | CrUrUrGrArArArArArGrUrGrGrCrArCrCrGrArGrUrCrGrGrU |
| | rGrCmU*mU*mU*rU-3′ |
| | (SEQ ID NO: 182) |
| CD33_guide_PC5 | 5′- |
| | mG*mG*mU*rGrG/iSpPC/rGrGrCrArGrCrUrGrArCrArArCrCr |
| | GrUrUrUrUrArGrArGrCrUrArGrArArArUrArGrCrArArGrUrU |
| | rArArArArUrArArGrGrCrUrArGrUrCrCrGrUrUrArUrCrArAr |
| | CrUrUrGrArArArArArGrUrGrGrCrArCrCrGrArGrUrCrGrGrU |
| | rGrCmU*mU*mU*rU-3′ |
| | (SEQ ID NO: 183) |
| CBE_BCL11A_PC5 | 5′- |
| | mU*mU*mU*rArU/iSpPC/rArCrArGrGrCrUrCrCrArGrGrArAr |
| | GrUrUrUrUrArGrArGrCrUrArGrArArArUrArGrCrArArGrUrU |
| | rArArArArUrArArGrGrCrUrArGrUrCrCrGrUrUrArUrCrArAr |
| | CrUrUrGrArArArArArGrUrGrGrCrArCrCrGrArGrUrCrGrGrU |
| | rGrCmU*mU*mU*rU-3′ |
| | (SEQ ID NO: 184) |

In Table 7 above, RNA is expressed as “r_”, 2′O-methyl RNA is expressed as “m_”, phosphorothioated RNA bases are expressed as “r_*”, phosphorothioated 2“O-methyl RNA bases are expressed as “m_*”, and internal photocleavable spacer is expressed as/iSpPC/.

As described herein, the location of the one or more photocleavable moiety within a gRNA may be based on factors such as being non-disruptive of the CRISPR/Cas nuclease activity in the absence of light (e.g., allows the CRISPR system to maintain on-target cleavage and not increase off-target effects), the ability to be cleaved in the presence of light (efficiency of cleavage in the location) including accessibility of the position to light, and inhibition efficiency (e.g. photocleavage at the position effectively eliminates or reduces cleavage activity). Also within the scope of the present disclosure is selection of one or more positions for a photocleavage moiety based on structural modeling or prediction of positions in the gRNA that are likely to be exposed/accessible to light adsorption.

For example, based on the predicted interaction between a Cpf1 nuclease and a gRNA, a number of surface exposed scaffold bases of the crRNA may be identified that are not expected to make significant contact with the target site sequence or Cpf1. In some embodiments, one or more nucleotides that may be surface exposed and accessible for light adsorption may be present in the stem-loop structure (handle) of the crRNA. For example, nucleotides at positions 7, 8, and 9 of the crRNA (corresponding to uracil at position 7, uracil at position 8, and cytosine at position 9 of the exemplary crRNA sequence provided by SEQ ID NO: 44), are predicted to be exposed for light adsorption. In some embodiments, a photocleavable moiety is located at one or more of positions 7, 8, and 9 of the crRNA.

Alternatively or in addition, one or more nucleotides of the targeting domain of the gRNA may be surface exposed and accessible for light adsorption. Disruption of one or more nucleotides of the targeting domain of the gRNA may also be predicted to eliminate or reduce interaction of the gRNA, the CRISPR/Cas nuclease, and/or the target site sequence. For example, nucleotides at positions 34, 36, and 37 of the gRNA (within the targeting domain) are predicted to be exposed for light adsorption. In some embodiments, a photocleavable moiety is located at one or more of positions of the targeting domain that are predicted to be surface exposed and accessible for light adsorption. In some embodiments, a photocleavable moiety is located at one or more of positions 34, 36, or 37 of the gRNA (within the targeting domain).

Table 8 provides an illustration of example photocleavable gRNA designs targeting C19 using a Cpf1 nuclease (e.g., AsCpf1). In Table 8, the “Guide Name” refers to the position number of the photocleavable moiety as the nucleotide position at which the moiety is located (i.e., the nucleotide position number that is replaced with the photocleavable moiety). For example, a photocleavable moiety at position 13 (pos13) is present in the gRNA following the nucleotide at position 12, meaning between the nucleotides at positions 11 and 13 of the gRNA, in place of the nucleotide at position 13.

TABLE 8

| Guide Name | gRNA Sequence (5′ to 3′) |
|---|---|
| Cpf1_CD19_PC_ | 5′- |
| pos13_handle_C-9 | UAAUUUCUACU/iSpPC/UUGUAGAUAGCGGGGACUCCCGAGACCAG- |
| | 3′ |
| | (SEQ ID NO: 185) |
| Cpf1_CD19_PC_ | 5′- |
| pos13_handle_U-8 | UAAUUUCUACUC/iSpPC/UGUAGAUAGCGGGGACUCCCGAGACCAG- |
| | 3′ |
| | (SEQ ID NO: 186) |
| Cpf1_CD19_PC_ | 5′- |
| pos14_handle_U-7 | UAAUUUCUACUCU/iSpPC/GUAGAUAGCGGGGACUCCCGAGACCAG- |
| | 3′ |
| | (SEQ ID NO: 187) |
| Cpf1_CD19_PC_ | 5′- |
| pos34 | UAAUUUCUACUCUUGUAGAUAGCGGGGACUCCC/iSpPC/AGACCAG- |
| | 3′ |
| | (SEQ ID NO: 188) |
| Cpf1_CD19_PC_ | 5′- |
| pos36 | UAAUUUCUACUCUUGUAGAUAGCGGGGACUCCCGA/iSpPC/ACCAG- |
| | 3′ |
| | (SEQ ID NO: 189) |
| Cpf1_CD19_PC_ | 5′- |
| pos37 | UAAUUUCUACUCUUGUAGAUAGCGGGGACUCCCGAG/iSpPC/CCAG- |
| | 3′ |
| | (SEQ ID NO: 190) |

In Table 8 above, the internal photocleavable spacer is expressed as/iSpPC/.

In some embodiments, any of the gRNAs provided herein comprise one or more nucleotides that are chemically modified. Chemical modifications of gRNAs have previously been described, and suitable chemical modifications include any modifications that are beneficial for gRNA function and do not measurably increase any undesired characteristics, e.g., off-target effects, of a given gRNA. Suitable chemical modifications include, for example, those that make a gRNA less susceptible to endo- or exonuclease catalytic activity, and include, without limitation, phosphorothioate backbone modifications, 2′-O-Me-modifications (e.g., at one or both of the 3′ and 5′ termini), 2′F-modifications, replacement of the ribose sugar with the bicyclic nucleotide-cEt, 3′thioPACE (MSP) modifications, or any combination thereof. Additional suitable gRNA modifications will be apparent to the skilled artisan based on this disclosure, and such suitable gRNA modifications include, without limitation, those described, e.g., in Rahdar et al. PNAS (2015) 112 (51) E7110-E7117 and Hendel et al., Nat Biotechnol. (2015); 33(9): 985-989, each of which is incorporated herein by reference in its entirety.

For example, a gRNA provided herein may comprise one or more 2′-O modified nucleotide, e.g., a 2′-O-methyl nucleotide. In some embodiments, the gRNA comprises a 2′-O modified nucleotide, e.g., 2′-O-methyl nucleotide at the 5′ end of the gRNA. In some embodiments, the gRNA comprises a 2′-O modified nucleotide, e.g., 2′-O-methyl nucleotide at the 3′ end of the gRNA. In some embodiments, the gRNA comprises a 2′-O-modified nucleotide, e.g., a 2′-O-methyl nucleotide at both the 5′ and 3′ ends of the gRNA. In some embodiments, the gRNA is 2′-O-modified, e.g. 2′-O-methyl-modified at the nucleotide at the 5′ end of the gRNA, the second nucleotide from the 5′ end of the gRNA, and the third nucleotide from the 5′ end of the gRNA. In some embodiments, the gRNA is 2′-O-modified, e.g., 2′-O-methyl-modified at the nucleotide at the 3′ end of the gRNA, the second nucleotide from the 3′ end of the gRNA, and the third nucleotide from the 3′ end of the gRNA. In some embodiments, the gRNA is 2′-O-modified, e.g., 2′-O-methyl-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified, e.g., 2′-O-methyl-modified at the second nucleotide from the 3′ end of the gRNA, the third nucleotide from the 3′ end of the gRNA, and at the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified, e.g., 2′-O-methyl-modified, at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA. In some embodiments, the 2′-O-methyl nucleotide comprises a phosphate linkage to an adjacent nucleotide. In some embodiments, the 2′-O-methyl nucleotide comprises a phosphorothioate linkage to an adjacent nucleotide. In some embodiments, the 2′-O-methyl nucleotide comprises a thioPACE linkage to an adjacent nucleotide.

In some embodiments, a gRNA provided herein may comprise one or more 2′-O-modified and 3′phosphorous-modified nucleotide, e.g., a 2′-O-methyl 3′ phosphorothioate nucleotide. In some embodiments, the gRNA comprises a 2′-O-modified and 3′ phosphorous-modified, e.g., 2′-O-methyl 3′ phosphorothioate nucleotide at the 5′ end of the gRNA. In some embodiments, the gRNA comprises a 2′-O-modified and 3′ phosphorous-modified, e.g., 2′-O-methyl 3′ phosphorothioate nucleotide at the 3′ end of the gRNA. In some embodiments, the gRNA comprises a 2′-O-modified and 3′ phosphorous-modified. e.g., 2′-O-methyl 3′ phosphorothioate nucleotide at the 5′ and 3′ ends of the gRNA. In some embodiments, the gRNA comprises a backbone in which one or more non-bridging oxygen atoms has been replaced with a sulfur atom. In some embodiments, the gRNA is 2′-O-modified and 3′ phosphorous-modified, e.g. 2′-O-methyl 3′ phosphorothioate-modified at the nucleotide at the 5′ end of the gRNA, the second nucleotide from the 5′ end of the gRNA, and the third nucleotide from the 5′ end of the gRNA. In some embodiments, the gRNA is 2′-O-modified and 3′ phosphorous-modified, e.g. 2′-O-methyl 3′ phosphorothioate-modified at the nucleotide at the 3′ end of the gRNA, the second nucleotide from the 3′ end of the gRNA, and the third nucleotide from the 3′ end of the gRNA. In some embodiments, the gRNA is 2′-O-modified and 3′ phosphorous-modified, e.g. 2′-O-methyl 3′ phosphorothioate-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified and 3′ phosphorous-modified. e.g. 2′-O-methyl 3′ phosphorothioate-modified at the second nucleotide from the 3′ end of the gRNA, the third nucleotide from the 3′ end of the gRNA, and the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified and 3′ phosphorous-modified. e.g. 2′-O-methyl 3′ phosphorothioate-modified at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA.

In some embodiments, a gRNA provided herein may comprise one or more 2′-O-modified and 3′-phosphorous-modified, e.g., 2′-O-methyl 3′thioPACE nucleotide. In some embodiments, the gRNA comprises a 2′-O-modified and 3′ phosphorous-modified, e.g., 2′-O-methyl 3′thioPACE nucleotide at the 5′ end of the gRNA. In some embodiments, the gRNA comprises a 2′-O-modified and 3′ phosphorous-modified, e.g., 2′-O-methyl 3′thioPACE nucleotide at the 3′ end of the gRNA. In some embodiments, the gRNA comprises a 2′-O-modified and 3′ phosphorous-modified, e.g., 2′-O-methyl 3′thioPACE nucleotide at the 5′ and 3′ ends of the gRNA. In some embodiments, the gRNA comprises a backbone in which one or more non-bridging oxygen atoms have been replaced with a sulfur atom and one or more non-bridging oxygen atoms have been replaced with an acetate group. In some embodiments, the gRNA is 2′-O-modified and 3′ phosphorous-modified, e.g. 2′-O-methyl 3′ thioPACE-modified at the nucleotide at the 5′ end of the gRNA, the second nucleotide from the 5′ end of the gRNA, and the third nucleotide from the 5′ end of the gRNA. In some embodiments, the gRNA is 2′-O-modified and 3′ phosphorous-modified, e.g. 2′-O-methyl 3′thioPACE-modified at the nucleotide at the 3′ end of the gRNA, the second nucleotide from the 3′ end of the gRNA, and the third nucleotide from the 3′ end of the gRNA. In some embodiments, the gRNA is 2′-O-modified and 3′ phosphorous-modified, e.g. 2′-O-methyl 3′thioPACE-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified and 3′ phosphorous-modified, e.g. 2′-O-methyl 3′thioPACE-modified at the second nucleotide from the 3′ end of the gRNA, the third nucleotide from the 3′ end of the gRNA, and the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified and 3′ phosphorous-modified, e.g. 2′-O-methyl 3′thioPACE-modified at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA.

In some embodiments, a gRNA provided herein comprises a chemically modified backbone. In some embodiments, the gRNA comprises a phosphorothioate linkage. In some embodiments, one or more non-bridging oxygen atoms have been replaced with a sulfur atom. In some embodiments, the nucleotide at the 5′ end of the gRNA, the second nucleotide from the 5′ end of the gRNA, and the third nucleotide from the 5′ end of the gRNA cach comprise a phosphorothioate linkage. In some embodiments, the nucleotide at the 3′ end of the gRNA, the second nucleotide from the 3′ end of the gRNA, and the third nucleotide from the 3′ end of the gRNA each comprise a phosphorothioate linkage. In some embodiments, the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA each comprise a phosphorothioate linkage. In some embodiments, the second nucleotide from the 3′ end of the gRNA, the third nucleotide from the 3′ end of the gRNA, and at the fourth nucleotide from the 3′ end of the gRNA each comprise a phosphorothioate linkage. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a phosphorothioate linkage.

In some embodiments, a gRNA provided herein comprises a thioPACE linkage. In some embodiments, the gRNA comprises a backbone in which one or more non-bridging oxygen atoms have been replaced with a sulfur atom and one or more non-bridging oxygen atoms have been replaced with an acetate group. In some embodiments, the nucleotide at the 5′ end of the gRNA, the second nucleotide from the 5′ end of the gRNA, and the third nucleotide from the 5′ end of the gRNA each comprise a thioPACE linkage. In some embodiments, the nucleotide at the 3′ end of the gRNA, the second nucleotide from the 3′ end of the gRNA, and the third nucleotide from the 3′ end of the gRNA each comprise a thioPACE linkage. In some embodiments, the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA each comprise a thioPACE linkage. In some embodiments, the second nucleotide from the 3′ end of the gRNA, the third nucleotide from the 3′ end of the gRNA, and at the fourth nucleotide from the 3′ end of the gRNA each comprise a thioPACE linkage. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a thioPACE linkage.

In some embodiments, a gRNA described herein comprises one or more 2′-O-methyl-3′-phosphorothioate nucleotides, e.g., at least 1, 2, 3, 4, 5, or 6 2′-O-methyl-3′-phosphorothioate nucleotides. In some embodiments, a gRNA described herein comprises modified nucleotides (e.g., 2′-O-methyl-3′-phosphorothioate nucleotides) at one or more of the three terminal positions and the 5′ end and/or at one or more of the three terminal positions and the 3′ end. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a 2′-O-methyl-3′-phosphorothioate nucleotides. In some embodiments, the gRNA may comprise one or more modified nucleotides, e.g., as described in PCT Publication Nos. WO2017/214460, WO2016/089433, and WO2016/164356, which are incorporated by reference their entirety.

The photocleavable gRNAs provided herein can be delivered to a cell in any manner suitable. Various suitable methods for the delivery of CRISPR/Cas systems, e.g., comprising an RNP including a gRNA bound to an RNA-guided nuclease, have been described, and exemplary suitable methods include, without limitation, electroporation of RNP into a cell, electroporation of mRNA encoding a Cas nuclease and a gRNA into a cell, various protein or nucleic acid transfection methods, and delivery of encoding RNA or DNA via viral vectors, such as, for example, retroviral (e.g., lentiviral) vectors. Any suitable delivery method is embraced by this disclosure, and the disclosure is not limited in this respect.

Prior to photocleavage, the photocleavable gRNAs described herein function as guide RNAs, e.g., bind and recruit a CRISPR/Cas nuclease to a target site sequence. In some embodiments, the photocleavable gRNAs described herein are capable of directing a CRISPR/Cas nuclease to a target site sequence and directing cleavage of one or both strands of DNA at the target site sequence. Following contact with light of a suitable wavelength, the photocleavable gRNAs are cleaved into two or more fragments, and preferably are not capable, or have reduce capacity, of directing CRISPR/Cas nuclease to the target site sequence or directing cleavage of one or both strains of DNA at the target site sequence.

Aspects of the present disclosure relate to methods of producing a CRISPR system, for example by contacting any of the photocleavable gRNAs described herein with a CRISPR/Cas nuclease.

In addition, aspects of the present disclosure provide methods of genetically engineering a cell, involving providing a cell and contacting the cell with any of the photocleavable gRNAs described herein and a CRISPR/Cas nuclease thus forming a ribonucleoprotein complex under conditions suitable for the gRNA to form and/or maintain an RNP complex with the CRISPR/Ca nuclease, thus forming a CRISPR system, and the RNP complex to bind a target site sequence in the genome of the cell. In some embodiments, the cell may be contacted with more than one photocleavable gRNA (e.g., photocleavable gRNAs having different sequence). For example, a cell may be contacted with a first photocleavable gRNA having a first sequence and second photocleavable gRNA having a second sequence. In some embodiments, the more than one photocleavable gRNAs may target distinct genetic loci (e.g., different genes or regions of genes).

In some embodiments, nuclease/gRNA complex (RNP complex) is formed, e.g., in vitro, and the cell is contacted with the RNP complex, e.g., via electroporation of the RNP complex into the cell. In some embodiments, the cell is contacted with CRISPR/Cas protein and gRNA separately, and the RNP complex is formed within the cell. In some embodiments, the cell is contacted with a nucleic acid, e.g., a DNA or RNA, encoding the CRISPR/Cas protein, and/or with a nucleic acid encoding the gRNA, or both. In some embodiments, the nucleic acid encoding the CRISPR/Cas protein and/or the nucleic acid encoding the gRNA is an mRNA or an mRNA analog.

Following contact of the cell with the gRNA and CRISPR/Cas nuclease, or RNP complex thereof, the cell may be incubated allowing for CRISPR/Cas nuclease activity to occur, e.g., binding and cleavage of at least one strand of DNA at the target site sequence in the genome of the cell. Cleavage by the CRISPR/Cas nuclease may be reduced or terminated at a desired time point by contacting the cell with light (e.g., provided by a light source) at a wavelength suitable to cleave the photocleavable moiety of the gRNA, resulting the generation of a plurality (e.g., 2 or more) of fragments of the gRNA. In some embodiments, cleavage of the photocleavable moiety of the gRNA reduces or eliminates the gRNA function of the photocleavable gRNA such that the CRISPR/Cas nuclease has reduced or absent recruitment to the target site sequence and/or the cleavage by the CRISPR/Cas nuclease at the target site sequence is reduced or eliminated.

In some embodiments, contacting the cell with light results in cleavage of at least 10%, 15%, 20%, 25%, 30%. 35%, 40%, 45%, 50%, 55%, 60%, 65%, 70%, 75%, 80%, 85%, 90%, 95%, or up to 100% of the photocleavable moiety. In some embodiments, contacting the cell with light results in cleavage of at least 10%, 15%, 20%, 25%. 30%, 35%, 40%, 45%, 50%, 55%. 60%, 65%, 70%, 75%, 80%, 85%, 90%, 95%, or up to 100% of gRNAs present in a cell are cleaved. In some embodiments, contacting the cell with light results at least 10%, 15%, 20%, 25%, 30%, 35%, 40%, 45%, 50%, 55%, 60%, 65%, 70%, 75%, 80%, 85%, 90%, 95%, or up to 100% of gRNAs present in a cell having reduced function or being non-functional (e.g., do not recruit the CRISPR/Cas nuclease and/or do not promote cleavage at the target site sequence).

In some embodiments, contacting the cell with light results in at least a 10%. 15%, 20%, 25%, 30%, 35%, 40%, 45%, 50%, 55%, 60%, 65%, 70%, 75%, 80%, 85%, 90%, 95%, or up to 100% reduction of cleavage by the CRISPR/Cas nuclease at the target site sequence. Methods of assessing functionality of a gRNA and cleavage by a CRISPR/Cas nuclease at a target site sequence may be performed for example using methods known in the art.

In some embodiments, contacting the cell with light results in at least a 10%, 15%, 20%, 25%, 30%, 35%, 40%, 45%, 50%, 55%, 60%, 65%, 70%, 75%, 80%, 85%, 90%, 95%, or up to 100% reduction of at least one gRNA activity. Any activity or function of a gRNA may be assessed to evaluate efficacy of the photocleavage, e.g., binding to a CRISPR/Cas, recruiting a CRISPR/Cas nuclease to the target site sequence, directing cleavage of the target site sequence.

In some embodiments, light has a wavelength from 300-380, 300-360, 300-350, 300-340, 300-320, 320-380, 320-360, 320-350, 320-340, 340-380, 340-360, 340-350, 350-380, 350-360, 350-375, or 360-380 nm. In some embodiments, light has a wavelength of about 365 nm. As described herein, selection of the wavelength of the light will depend on factors, such as the photocleavable moiety present in the gRNA and potential effects on the target cell.

In some embodiments, the cell is contacted with light for a duration of time sufficient to achieve a desired level of photocleavage while minimizing any potential adverse effects on the target cell. In some embodiments, the cell is contacted with light for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 90, or 120 seconds. In some embodiments, the cell is contacted with light for about 30 seconds.

Also within the scope of the present disclosure are cells containing more than one photocleavable gRNAs, with each photocleavable gRNAs targeting different genetic loci. In some embodiments, each of the photocleavable gRNAs may contain different photocleavable moieties, such that contacting the cell with light having a first wavelength results in cleavage of one of the photocleavable gRNAs, while the second photocleavable gRNA may remain intact and capable of cleavage at the corresponding target site. In such instances, contacting the cell with light having a second wavelength may result in cleavable of the second photocleavable gRNA. In some embodiments, each of the photocleavable gRNAs may contain the photocleavable moieties or different photocleavable moiety capable of being cleaved at the same (or similar) wavelength, such that contacting the cell with light having said wavelength results in cleavage of both photocleavable gRNAs.

The compositions and methods provided herein may be applied to any cell or cell type capable of being genetically engineered using the photocleavable gRNAs and methods described herein. In some embodiments, the cell is a eukaryotic cell. In some embodiments, the cell is a mammalian cell, yeast cell, fungal cell, or plant cell. In some embodiments, the cell is a human cell or a mouse cell. In some embodiments, the cells may be obtained from a subject, such as a human subject. Methods of obtaining cells, such as hematopoietic stem cells are described, e.g., in PCT Application No. PCT/US2016/057339, which is herein incorporated by reference in its entirety. In some embodiments, the mammalian subject is a non-human primate, a rodent (e.g., mouse or rat), a bovine, a porcine, an equine, or a domestic animal.

In some embodiments, the cells provided herein are hematopoietic cells, e.g., hematopoietic stem cells (HSC) or hematopoietic progenitor cells (HPC). In some embodiments, the cells provided herein hematopoietic stem or progenitor cells. Hematopoietic stem cells (HSCs) are typically capable of giving rise to both myeloid and lymphoid progenitor cells that further give rise to myeloid cells (e.g., monocytes, macrophages, neutrophils, basophils, dendritic cells, erythrocytes, platelets, etc.) and lymphoid cells (e.g., T cells, B cells, NK cells), respectively. HSCs are characterized by the expression of the cell surface marker CD34 (e.g., CD34+), which can be used for the identification and/or isolation of HSCs, and absence of cell surface markers associated with commitment to a cell lineage. In some embodiments, the HSCs are peripheral blood HSCs.

In some embodiments, the cells provided herein are immune effector cells. In some embodiments, the immune effector cell is a lymphocyte. In some embodiments, the immune effector cell is a T-lymphocyte. In some embodiments, the T-lymphocyte is an alpha/beta T-lymphocyte. In some embodiments, the T-lymphocyte is a gamma/delta T-lymphocyte. In some embodiments, the immune effector cell is a natural killer T (NKT cell). In some embodiments, the immune effector cell is a natural killer (NK) cell.

The skilled artisan will understand, however, that the provision of such examples is for the purpose of illustrating some specific embodiments, and additional suitable cells and cell types will be apparent to the skilled artisan based on the present disclosure, which is not limited in this respect.

In some embodiments, a genetically engineered cell provided herein comprises only one genomic modification, e.g., a genomic modification that results in a loss of expression of a protein, for example a protein encoded by or regulated by the target site sequence, or expression of a variant form of the protein. It will be understood that the gene editing methods provided herein may result in genomic modifications in one or both alleles of a target genetic loci. In some embodiments, genetically engineered cells comprising a genomic modification in both alleles of a given genetic locus are preferred.

In some embodiments, a genetically engineered cell provided herein comprises two or more genomic modifications. For example, a population of genetically engineered cells can comprise a plurality of different mutations.

As will be evident to one of ordinary skill in the art, the compositions and methods described herein may be used to modify any genetic locus in a cell, including for example protein-coding, non-protein coding, chromosomal, and extra-chromosomal sequences. Accordingly, targeting domains of the photocleavable gRNAs may be designed to target any genetic locus (i.e., a target site sequence), such as a target site sequence adjacent to a PAM sequence for a corresponding CRISPR/Cas nuclease.

In some embodiments, the targeting domain targets a cell surface protein, such as a Type 0, Type 1, or Type 2 cell surface protein. In some embodiments, the targeting domain targets BCMA. CD19, CD20, CD30, ROR1, B7H6, B7H3, CD23, CD33, CD38. C-type lectin like molecule-1, CS1. IL-5, L1-CAM, PSCA, PSMA, CD138, CD133, CD70, CD5, CD6, CD7, CD13, NKG2D, NKG2D ligand, CLEC12A, CD11, CD123, CD56, CD34, CD14, CD66b, CD41, CD61, CD62, CD235a, CD146, CD326. LMP2, CD22, CD52, CD10, CD3/TCR. CD79/BCR, and/or CD26.

In some embodiments, the targeting domain targets a cell surface protein associated with a neoplastic or malignant disease or disorder, e.g., with a specific type of cancer, such as, without limitation, CD20, CD22 (Non-Hodgkin's lymphoma, B-cell lymphoma, chronic lymphocytic leukemia (CLL)), CD52 (B-cell CLL), CD33 (Acute myelogenous leukemia (AML)), CD10 (gp100) (Common (pre-B) acute lymphocytic leukemia and malignant melanoma), CD3/T-cell receptor (TCR) (T-cell lymphoma and leukemia), CD79/B-cell receptor (BCR) (B-cell lymphoma and leukemia), CD26 (epithelial and lymphoid malignancies), human leukocyte antigen (HLA)-DR. HLA-DP, and HLA-DQ (lymphoid malignancies), RCAS1 (gynecological carcinomas, biliary adenocarcinomas and ductal adenocarcinomas of the pancreas) as well as prostate specific membrane antigen.

Additional non-limiting examples of cell surface proteins include CD1a, CD1b, CD1c, CD1d, CD1e, CD2, CD3, CD3d, CD3e, CD3g, CD4, CD5, CD6, CD7, CD8a, CD8b, CD9, CD10, CD11a, CD11b, CD11c, CD11d, CDw12, CD13, CD14, CD15, CD16, CD16b, CD17, CD18, CD19, CD20, CD21, CD22, CD23, CD24, CD25, CD26, CD27, CD28, CD29, CD30, CD31, CD32a, CD32b, CD32c, CD34, CD35, CD36, CD37, CD38, CD39, CD40, CD41, CD42a, CD42b, CD42c. CD42d, CD43, CD44, CD45, CD45RA. CD45RB, CD45RC, CD45RO, CD46, CD47, CD48, CD49a, CD49b, CD49c, CD49d, CD49c, CD49f, CD50, CD51, CD52, CD53, CD54, CD55, CD56, CD57, CD58, CD59, CD60a, CD61, CD62E, CD62L, CD62P, CD63, CD64a, CD65, CD65s, CD66a, CD66b, CD66c. CD66F, CD68, CD69, CD70, CD71, CD72, CD73, CD74, CD75, CD75S, CD77, CD79a. CD79b, CD80, CD81, CD82, CD83, CD84, CD85A, CD85C, CD85D, CD85E, CD85F, CD85G, CD85H, CD85I, CD85J, CD85K, CD86, CD87, CD88, CD89, CD90, CD91, CD92, CD93, CD94, CD95, CD96, CD97, CD98, CD99, CD99R, CD100, CD101, CD102, CD103, CD104, CD105, CD106, CD107a, CD107b, CD108. CD109, CD110, CD111, CD112, CD113, CD114, CD115, CD116, CD117, CD118, CD119, CD120a, CD120b, CD121a, CD121b, CD121a, CD121b, CD122, CD123, CD124, CD125, CD126, CD127, CD129, CD130, CD131, CD132, CD133, CD134, CD135, CD136, CD137, CD138, CD139, CD140a. CD140b. CD141, CD142, CD143, CD14. CDw145, CD146, CD147. CD148, CD150, CD152, CD152, CD153, CD154, CD155, CD156a, CD156b, CD156c, CD157, CD158b1, CD158b2, CD158d, CD158el/e2, CD158f, CD158g, CD158h, CD158i, CD158j, CD158k. CD159a. CD159c. CD160, CD161, CD163, CD164, CD165, CD166, CD167a. CD168. CD169, CD170, CD171, CD172a, CD172b, CD172g, CD173, CD174, CD175, CD175s, CD176, CD177, CD178, CD179a, CD179b, CD180, CD181, CD182, CD183, CD184, CD185, CD186, CD191, CD192, CD193, CD194, CD195, CD196, CD197. CDw198. CDw199, CD200, CD201. CD202b, CD203c, CD204, CD205, CD206, CD207, CD208, CD209, CD210a, CDw210b, CD212, CD213a1, CD213a2, CD215, CD217, CD218a, CD218b, CD220, CD221, CD222, CD223, CD224, CD225, CD226, CD227, CD228, CD229, CD230, CD231, CD232, CD233. CD234, CD235a. CD235b, CD236, CD236R, CD238, CD239, CD240, CD241, CD242, CD243, CD244, CD245, CD246, CD247, CD248, CD249, CD252, CD253, CD254, CD256, CD257, CD258, CD261, CD262, CD263, CD264, CD265, CD266, CD267, CD268, CD269, CD270. CD272, CD272, CD273, CD274, CD275, CD276, CD277, CD278, CD279, CD280, CD281, CD282, CD283, CD284, CD286, CD288, CD289, CD290, CD292, CDw293, CD294, CD295, CD296, CD297, CD298, CD299, CD300a, CD300c, CD300e, CD301, CD302, CD303, CD304, CD305, CD306, CD307a, CD307b, CD307c, CD307d, CD307e, CD309, CD312, CD314, CD315, CD316, CD317, CD318, CD319, CD320, CD321, CD322, CD324, CD325, CD326, CD327, CD328, CD329, CD331, CD332, CD333, CD334, CD335, CD336, CD337, CD338, CD339, CD340, CD344, CD349, CD350, CD351, CD352, CD353, CD354, CD355, CD357, CD358, CD359, CD360, CD361, CD362, and CD363.

Some of the embodiments, advantages, features, and uses of the technology disclosed herein will be more fully understood from the Examples below. The Examples are intended to illustrate some of the benefits of the present disclosure and to describe particular embodiments but are not intended to exemplify the full scope of the disclosure and, accordingly, do not limit the scope of the disclosure.

### Examples

### Example 1. Generation of Genetically Engineered Hematopoietic Cells Using Cas12a, Cas9, and Photocleavable gRNAs

This example demonstrates generation of photocleavable gRNAs and their use in generating genetically engineered hematopoietic cells.

Exemplary Cas12a gRNAs and Cas9 sgRNAs are indicated in Tables 1-8, respectively, and were designed based on the Cas12a PAM (5′-NTTN-3′) and Cas9 PAM (5′-NGG-3′) with close proximity to exemplary target regions and evaluated for predicted specificity by minimizing potential off-target sites in the human genome with an online search algorithm (e.g., the Benchling algorithm, Doench et al 2016, Hsu et al 2013). Exemplary target regions, the genes encoding CD19, CD5, CD33, VEGFA, and BCL11A are targeted by the exemplary gRNAs and sgRNAs below.

Cas12a gRNAs are synthesis using gRNA target domains directed to human CD19 and the Cas12a gRNA scaffold sequence (crRNA) 5′-UAAUUUCUACUCUUGUAGAU-3′ (SEQ ID NO: 44).

Cas9 sgRNAs are synthesized using the gRNA targeting domains provided below and the Cas9 sgRNA scaffold sequence 5′-GUUUUAGAGCUAGAAAUAGCAAGUUAAAAUAAGGCUAGUCCGUUAUCAACUUGA AAAAGUGGCA CCGAGUCGGUGCUUUU-3′ (SEQ ID NO: 191).

gRNAs are synthesized with an exemplary photocleavable moiety comprising an o-nitrobenzyl moiety at various indicated positions, as shown in Tables 1-8. Exposure to an appropriate wavelength of radiation (i.e., 365 nm for 30 seconds) causes the photocleavable moiety to undergo photochemistry that cleaves the gRNA at least two nucleic acid fragments, thereby reducing or eliminating the ability of the gRNA to recruit and direct Cas nuclease cleavage at the target site sequence.

Peripheral blood mononuclear cells are collected from healthy donor subject by apheresis following hematopoietic stem cell mobilization. Alternatively, frozen CD34+HSCs derived from mobilized peripheral blood (mPB) are purchased, for example, from Hemacare or Fred Hutchinson Cancer Center and thawed according to manufacturer's instructions. ˜1×10⁶ HSCs are thawed and cultured in StemSpan SFEM medium supplemented with StemSpan CC110 cocktail (StemCell Technologies) for 24-48 h before electroporation with RNP.

The donor or purchased CD34+ cells are electroporated with Cas12a protein and any of the indicated Cas12a gRNAs disclosed herein (e.g., Tables 2, 5, 6, and 8) or Cas9 protein and any of the indicated Cas9 sgRNAs disclosed herein (e.g., Tables 3 and 7). To electroporate HSCs, 1.5×10⁵cells are pelleted and resuspended in 20 μL Lonza P3 solution and mixed with 10 μL RNP. CD34+HSCs are electroporated using the Lonza Nucleofector 2 and the Human P3 Cell Nucleofection Kit (VPA-1002, Lonza). Cas9 and Cas12a protein may be purchased from Synthego.

The edited cells are cultured for less than 48 hours. At varying times during culture (e.g., at 0, 0.5, 1, 2, 3, 4, 5, 6, 7, 8, 9, 10, 11, 12, 15, 18, 21, 24, 27, 30, 33, 36, 39, 42, 45, and/or 48 hours post-electroporation), cell aliquots are harvested and exposed to radiation at an absorbance peak appropriate for the photocleavable moiety (e.g., 360 nm, e.g., for an o-nitrobenzyl moiety). Upon harvest, the cells are washed, resuspended in the final formulation, and cryopreserved.

A representative sample of the edited HSCs (e.g., a portion of or all cells of the time point aliquots) is evaluated for viability and expression of exemplary target region genes (e.g., CD19, CD5, CD33, and/or VEGFA), or absence thereof, by staining using target-specific antibody and analyzed by flow cytometry. Edited HSC populations exhibiting at least 70% cell viability and at least 45% editing efficiency (i.e., absence of target gene expression in at least 45% of the cells in the cell population) at 48 hours after electroporation may be used in downstream applications, e.g., for HCT.

### Genomic DNA Analysis

For all genomic analysis, DNA is harvested from cells, amplified with primers flanking the target region, purified and the allele modification frequencies are analyzed using TIDE (Tracking of Indels by Decomposition). Analyses are performed using a reference sequence from a mock-transfected sample. Parameters are set to the default maximum indel size of 10 nucleotides and the decomposition window to cover the largest possible window with high quality traces. All TIDE analyses below the detection sensitivity of 3.5% are set to 0%. The percentage editing is determined by % INDEL as assessed by TIDE analysis. Editing efficiency is determined by flow cytometric analysis.

### Flow Cytometry Analysis

The gRNA-edited cells may also be evaluated for surface expression of target gene encoded protein, for example by flow cytometry analysis (FACS). Live CD34+ HSCs are stained for target gene protein using a target-specific antibody and analyzed by flow cytometry on the Attune N×T flow cytometer (Life Technologies). Cells in which the target gene have been genetically modified show a reduction in target gene protein expression as detected by FACS.

### Viability of Edited Cells

At 0, 0.5, 1, 2, 3, 4, 5, 6, 7, 8, 9, 10, 11, 12, 15, 18, 21, 24, 27, 30, 33, 36, 39, 42, 45, and/or 48 hours post-ex vivo editing (e.g., 4, 24, and 48 hours post-ex vivo editing), the percentages of viable, edited cells and control cells are quantified using flow cytometry and the 7AAD viability dye. Cells edited using the exemplary gRNAs or sgRNAs described herein may be viable and remain viable over time following electroporation, gene editing, radiation exposure, and photocleavage. This is similar to what is observed in the control mock edited cells.

### REFERENCES

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

### Equivalents and Scope

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an.” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods described herein, can be excluded from any one or more claims. For purposes of brevity, all of the embodiments in which one or more elements, features, purposes, or aspects is excluded are not set forth explicitly herein.

What is claimed is:

1. A guide RNA (gRNA) comprising:
a targeting domain;
a CRISPR RNA (crRNA) sequence for a CRISPR/Cas nuclease; and
at least one photocleavable moiety.

2. The gRNA of claim 1, wherein the gRNA comprises 1, 2, 3, 4, 5, or more photocleavable moieties.

3. The gRNA of claim 1 or 2, wherein the photocleavable moiety is a 2-nitrobenzyl bond.

4. The gRNA of any one of claims 1-3, wherein the targeting domain is corresponding to a target site sequence adjacent to a protospacer-adjacent motif (PAM) in a gene.

5. The gRNA of any one of claims 1-4, wherein the crRNA sequence comprises a first complementarity domain, a linking domain, and a second complementarity domain that is complementary to the first complementarity domain; and a proximal domain.

6. The gRNA of any one of claims 1-5, wherein the at least one photocleavable moiety is located within the targeting domain, wherein the photocleavable moiety is located between two nucleotides of the targeting domain at a position corresponding to a nucleotide of the target site sequence.

7. The gRNA of claim 6, wherein the at least one photocleavable moiety is located following nucleotide position 1, 2, 3, 4, 5, 6, 7, 8, 9, 10, 11, 12, 13, 14, 15, 16, 17, 18, 19, 20, 21, or 22 from the 5′ end of the targeting domain.

8. The gRNA of claim 6 or 7, wherein the at least one photocleavable moiety is located following nucleotide position 1, 2, 3, 4, 5, 6, 7, 8, 9, 10, 11, 12, 13, 14, 15, 16, 17, 18, 19, 20, 21, or 22 from the 5′ end of the gRNA.

9. The gRNA of any one of claims 6-8, wherein the at least one photocleavable moiety is located at any one or more of positions 9, 11, 13, 15, or 17 from the 5′ end of the targeting domain.

10. The gRNA of any one of claims 6-9, wherein the at least one photocleavable moiety is located at any one or more of positions 9, 11, 13, 15, or 17 from the 5′ end of the gRNA.

11. The gRNA of any one of claims 6-10, wherein the at least one photocleavable moiety is located between nucleotide positions 1 and 3, 2 and 4, 3 and 5, 4 and 6, 5 and 7, 6 and 8, 7 and 9, 8 and 10, 9 and 11, 10 and 12, 11 and 13, 12 and 14, 13 and 15, 14 and 16, 15 and 17, 16 and 18, 17 and 19, 18 and 20, 19 and 21, or 20 and 22 from the 5′ end of the targeting domain.

12. The gRNA of any one of claims 6-11, wherein the at least one photocleavable moiety is located between nucleotide positions 1 and 3, 2 and 4, 3 and 5, 4 and 6, 5 and 7, 6 and 8, 7 and 9, 8 and 10, 9 and 11, 10 and 12, 11 and 13, 12 and 14, 13 and 15, 14 and 16, 15 and 17, 16 and 18, 17 and 19, 18 and 20, 19 and 21, or 20 and 22 from the 5′ end of the gRNA.

13. The gRNA of any one of claims 6-12, wherein the at least one photocleavable moiety is located within 5, 10, or 15 nucleotides from the 3′ end of the targeting domain.

14. The gRNA of any one of claims 6-13, wherein the at least one photocleavable moiety is located within the targeting domain following a position corresponding to the nucleotide at a position 1, 2, 3, 4, 5, 6, 7, 8, 9, 10, 11, 12, 13, 14, 15, 16, 17, 18, 19, 20, 21, or 22 nucleotides adjacent to the PAM.

15. The gRNA of any one of claims 6-14, wherein the at least one photocleavable moiety is located within the targeting domain between nucleotide positions 1 and 3, 2 and 4, 3 and 5, 4 and 6, 5 and 7, 6 and 8, 7 and 9, 8 and 10, 9 and 11, 10 and 12, 11 and 13, 12 and 14, 13 and 15, 14 and 16, 15 and 17, 16 and 18, 17 and 19, 18 and 20, 19 and 21, 20 and 22, or 21 and 23 adjacent to the PAM.

16. The gRNA of claim 14, wherein the at least one photocleavable moiety is located within the targeting domain following a position corresponding to the nucleotide at one or more of positions 9, 11, 13, 15, or 17 nucleotides adjacent to the PAM.

17. The gRNA of any one of claims 1-16, wherein the at least one photocleavable moiety is located within the crRNA sequence, wherein the photocleavable moiety is located between two nucleotides of the cRNA sequence replacing a nucleotide of a corresponding wildtype crRNA sequence.

18. The gRNA of claim 17, wherein the at least one photocleavable moiety is located following nucleotide position 1, 2, 3, 4, 5, 6, 7, 8, 9, 10, 11, 12, 13, 14, 15, 16, 17, 18, 19, or 20 from the 5′ end of the crRNA sequence.

19. The gRNA of claim 17, wherein the at least one photocleavable moiety is located following nucleotide position 1, 2, 3, 4, 5, 6, 7, 8, 9, 10, 11, 12, 13, 14, 15, 16, 17, 18, 19, or 20 from the 5′ end of the gRNA.

20. The gRNA of any one of claims 17-19, wherein the at least one photocleavable moiety is located within 5, 10, or 15 nucleotides from the 3′ end of the crRNA sequence.

21. The gRNA of any one of claims 17-20, wherein the at least one photocleavable moiety is located within 5, 10, or 15 nucleotides from the 3′ end of the gRNA.

22. The gRNA of any one of claims 17-21, wherein the at least one photocleavable moiety is located between nucleotide positions 1 and 3, 2 and 4, 3 and 5, 4 and 6, 5 and 7, 6 and 8, 7 and 9, 8 and 10, 9 and 11, 10 and 12, 11 and 13, 12 and 14, 13 and 15, 14 and 16, 15 and 17, 16 and 18, 17 and 19, 18 and 20, 19 and 21 from the 5′ end of the crRNA sequence.

23. The gRNA of any one of claims 17-22, wherein the at least one photocleavable moiety is located between nucleotide positions 1 and 3, 2 and 4, 3 and 5, 4 and 6, 5 and 7, 6 and 8, 7 and 9, 8 and 10, 9 and 11, 10 and 12, 11 and 13, 12 and 14, 13 and 15, 14 and 16, 15 and 17, 16 and 18, 17 and 19, 18 and 20, or 19 and 21 from the 5′ end of the gRNA.

24. The gRNA of any one of claims 1-23, wherein the CRISPR/Cas nuclease is a Cas12a (Cpf1) nuclease.

25. The gRNA of claim 24, wherein the Cas12a nuclease is AsCas12a, LbCas12a, FnCas12a, or PaCas 12a nuclease.

26. The gRNA of any one of claims 1-23, wherein the CRISPR/Cas nuclease is a Cas9 nuclease.

27. The gRNA of claim 26, wherein the gRNA is a single guide RNA (sgRNA).

28. The gRNA of claim 27, wherein at least one photocleavable moiety is located within 10, 15, or 20 nucleotides of the 5′ end of the sgRNA.

29. The gRNA of any of claims 1-28, wherein the gRNA comprises one or more nucleotide residues that are chemically modified.

30. The gRNA of any of claims 1-29, wherein the gRNA comprises one or more nucleotide residues that comprise a 2′O-methyl moiety.

31. The gRNA of any of claims 1-30, wherein the gRNA comprises one or more nucleotide residues that comprise a phosphorothioate.

32. The gRNA of any of claims 1-31, wherein the gRNA comprises one or more nucleotide residues that comprise a thioPACE moiety.

33. The gRNA of any one of claims 1-32, wherein the gRNA is capable of directing the CRISPR nuclease to a target site sequence.

34. The gRNA of any one of the preceding claims, wherein the gRNA comprises a sequence of any one of SEQ ID NOs: 1-40.

35. The gRNA of any one of the preceding claims, wherein the wherein the gRNA comprises a sequence of any one of SEQ ID NO: 41-43.

36. The gRNA of any one of the preceding claims, wherein the gRNA comprises a sequence of any one of SEQ ID NOs: 134-172, 174, 176, 178, and 180.

37. The gRNA of any one of the preceding claims, wherein the wherein the gRNA comprises a sequence of any one of SEQ ID NOs: 181-190.

38. A CRISPR system comprising a CRISPR/Cas nuclease and the gRNA of any one of claims 1-37.

39. The CRISPR system of claim 38, wherein the CRISPR/Cas nuclease is a Cas12a (Cpf1) nuclease.

40. The CRISPR system of claim 39, wherein the Cas 12a nuclease is AsCas12a, LbCas12a, FnCas12a, or PaCas12a nuclease.

41. The CRISPR system of claim 38, wherein the CRISPR/Cas nuclease is a Cas9 nuclease.

42. A method, comprising contacting the gRNA of any one of claims 1-37 and a CRISPR/Cas nuclease to form a CRISPR system.

43. The method of claim 42, wherein the CRISPR/Cas nuclease is a Cas12a (Cpf1) nuclease.

44. The method of claim 43, wherein the Cas12a nuclease is AsCas12a, LbCas 12a, FnCas12a, or PaCas12a nuclease.

45. The method of claim 42, wherein the CRISPR/Cas nuclease is a Cas9 nuclease.

46. A method, comprising
a. providing a cell, and
b. contacting a cell with (i) the gRNA of any one of claims 1-37 and (ii) a CRISPR/Cas nuclease, thus forming a ribonucleoprotein (RNP) complex under conditions suitable for the gRNA of (i) to form and/or maintain an RNP complex with the CRISPR/Cas nuclease of (ii), thus forming a CRISPR system, and for the RNP complex to bind a target site sequence in the genome of the cell.

47. The method of claim 46, wherein the contacting comprises introducing (i) and (ii) into the cell in the form of a pre-formed ribonucleoprotein (RNP) complex.

48. The method of claim 46, wherein the contacting comprises introducing (i) and/or (ii) into the cell in the form of a nucleic acid encoding the gRNA of (i) and/or the CRISPR/Cas nuclease of (ii).

49. The method of claim 48, wherein the nucleic acid encoding the gRNA of (i) and/or the CRISPR nuclease of (ii) is an RNA, preferably an mRNA or an mRNA analog.

50. The method of claim 47, wherein the pre-formed RNP complex is introduced into the cell via electroporation.

51. The method of any one of claims 46-50, wherein the method further comprises contacting the cell with light such that at least 50% of the photocleavable moiety is cleaved.

52. The method of any one of claims 46-51, wherein the method further comprises contacting the cell with light resulting in at least a 50% reduction of cleavage by the CRISPR/Cas nuclease at the target site sequence.

53. The method of claim 51 or 52, wherein the light has a wavelength of about 350-375 nm.

54. The method of claim 53, wherein the light has a wavelength of about 365 nm.

55. The method of any one of claims 46-54, wherein the cell is contacted with light for between 10 seconds and 60 seconds.

56. The method of claim 55, wherein the cell is contacted with light for about 30 seconds.

57. The method of any one of claims 46-56, wherein the cell is a hematopoietic cell.

58. The method of any one of claims 46-57, wherein the cell is a hematopoietic stem cell.

59. The method of any one of claims 46-58, wherein the cell is a hematopoietic progenitor cell.

60. The method of any one of claims 46-56, wherein the cell is an immune effector cell.

61. The method of any one of claim 46-56 or 60, wherein the cell is a lymphocyte.

62. The method of any one of claim 46-56, 60, or 61, wherein the cell is a T-lymphocyte.

63. A genetically engineered cell, wherein the cell is obtained by the method of any of claims 46-62.

64. A cell population, comprising the genetically engineered cell of claim 63.


SEQUENCE LISTING (193 sequences)

SEQ ID NO: 1 - RNA, 41 nt, Artificial Sequence
  Synthetic
  misc_feature (1)..(2): May be separated by a photocleavable moiety. In some instances, the photocleavable moitey is a 2-nitrobenzyl bond.
uaauuucuacucuuguagauagcggggacucccgagaccag

SEQ ID NO: 2 - RNA, 41 nt, Artificial Sequence; identical to SEQ ID NO: 1
  Synthetic
  misc_feature (2)..(3): May be separated by a photocleavable moiety. In some instances, the photocleavable moitey is a 2-nitrobenzyl bond.

SEQ ID NO: 3 - RNA, 41 nt, Artificial Sequence; identical to SEQ ID NO: 1
  Synthetic
  misc_feature (3)..(4): May be separated by a photocleavable moiety. In some instances, the photocleavable moitey is a 2-nitrobenzyl bond.

SEQ ID NO: 4 - RNA, 41 nt, Artificial Sequence; identical to SEQ ID NO: 1
  Synthetic
  misc_feature (4)..(5): May be separated by a photocleavable moiety. In some instances, the photocleavable moitey is a 2-nitrobenzyl bond.

SEQ ID NO: 5 - RNA, 41 nt, Artificial Sequence; identical to SEQ ID NO: 1
  Synthetic
  misc_feature (5)..(6): May be separated by a photocleavable moiety. In some instances, the photocleavable moitey is a 2-nitrobenzyl bond.

SEQ ID NO: 6 - RNA, 41 nt, Artificial Sequence; identical to SEQ ID NO: 1
  Synthetic
  misc_feature (6)..(7): May be separated by a photocleavable moiety. In some instances, the photocleavable moitey is a 2-nitrobenzyl bond.

SEQ ID NO: 7 - RNA, 41 nt, Artificial Sequence; identical to SEQ ID NO: 1
  Synthetic
  misc_feature (7)..(8): May be separated by a photocleavable moiety. In some instances, the photocleavable moitey is a 2-nitrobenzyl bond.

SEQ ID NO: 8 - RNA, 41 nt, Artificial Sequence; identical to SEQ ID NO: 1
  Synthetic
  misc_feature (8)..(9): May be separated by a photocleavable moiety. In some instances, the photocleavable moitey is a 2-nitrobenzyl bond.

SEQ ID NO: 9 - RNA, 41 nt, Artificial Sequence; identical to SEQ ID NO: 1
  Synthetic
  misc_feature (9)..(10): May be separated by a photocleavable moiety. In some instances, the photocleavable moitey is a 2-nitrobenzyl bond.

SEQ ID NO: 10 - RNA, 41 nt, Artificial Sequence; identical to SEQ ID NO: 1
  Synthetic
  misc_feature (10)..(11): May be separated by a photocleavable moiety. In some instances, the photocleavable moitey is a 2-nitrobenzyl bond.

SEQ ID NO: 11 - RNA, 41 nt, Artificial Sequence; identical to SEQ ID NO: 1
  Synthetic
  misc_feature (11)..(12): May be separated by a photocleavable moiety. In some instances, the photocleavable moitey is a 2-nitrobenzyl bond.

SEQ ID NO: 12 - RNA, 41 nt, Artificial Sequence; identical to SEQ ID NO: 1
  Synthetic
  misc_feature (12)..(13): May be separated by a photocleavable moiety. In some instances, the photocleavable moitey is a 2-nitrobenzyl bond.

SEQ ID NO: 13 - RNA, 41 nt, Artificial Sequence; identical to SEQ ID NO: 1
  Synthetic
  misc_feature (13)..(14): May be separated by a photocleavable moiety. In some instances, the photocleavable moitey is a 2-nitrobenzyl bond.

SEQ ID NO: 14 - RNA, 41 nt, Artificial Sequence; identical to SEQ ID NO: 1
  Synthetic
  misc_feature (14)..(15): May be separated by a photocleavable moiety. In some instances, the photocleavable moitey is a 2-nitrobenzyl bond.

SEQ ID NO: 15 - RNA, 41 nt, Artificial Sequence; identical to SEQ ID NO: 1
  Synthetic
  misc_feature (15)..(16): May be separated by a photocleavable moiety. In some instances, the photocleavable moitey is a 2-nitrobenzyl bond.

SEQ ID NO: 16 - RNA, 41 nt, Artificial Sequence; identical to SEQ ID NO: 1
  Synthetic
  misc_feature (16)..(17): May be separated by a photocleavable moiety. In some instances, the photocleavable moitey is a 2-nitrobenzyl bond.

SEQ ID NO: 17 - RNA, 41 nt, Artificial Sequence; identical to SEQ ID NO: 1
  Synthetic
  misc_feature (17)..(18): May be separated by a photocleavable moiety. In some instances, the photocleavable moitey is a 2-nitrobenzyl bond.

SEQ ID NO: 18 - RNA, 41 nt, Artificial Sequence; identical to SEQ ID NO: 1
  Synthetic
  misc_feature (18)..(19): May be separated by a photocleavable moiety. In some instances, the photocleavable moitey is a 2-nitrobenzyl bond.

SEQ ID NO: 19 - RNA, 41 nt, Artificial Sequence; identical to SEQ ID NO: 1
  Synthetic
  misc_feature (19)..(20): May be separated by a photocleavable moiety. In some instances, the photocleavable moitey is a 2-nitrobenzyl bond.

SEQ ID NO: 20 - RNA, 41 nt, Artificial Sequence; identical to SEQ ID NO: 1
  Synthetic
  misc_feature (20)..(21): May be separated by a photocleavable moiety. In some instances, the photocleavable moitey is a 2-nitrobenzyl bond.

SEQ ID NO: 21 - RNA, 41 nt, Artificial Sequence; identical to SEQ ID NO: 1
  Synthetic
  misc_feature (21)..(22): May be separated by a photocleavable moiety. In some instances, the photocleavable moitey is a 2-nitrobenzyl bond.

SEQ ID NO: 22 - RNA, 41 nt, Artificial Sequence; identical to SEQ ID NO: 1
  Synthetic
  misc_feature (22)..(23): May be separated by a photocleavable moiety. In some instances, the photocleavable moitey is a 2-nitrobenzyl bond.

SEQ ID NO: 23 - RNA, 41 nt, Artificial Sequence; identical to SEQ ID NO: 1
  Synthetic
  misc_feature (23)..(24): May be separated by a photocleavable moiety. In some instances, the photocleavable moitey is a 2-nitrobenzyl bond.

SEQ ID NO: 24 - RNA, 41 nt, Artificial Sequence; identical to SEQ ID NO: 1
  Synthetic
  misc_feature (24)..(25): May be separated by a photocleavable moiety. In some instances, the photocleavable moitey is a 2-nitrobenzyl bond.

SEQ ID NO: 25 - RNA, 41 nt, Artificial Sequence; identical to SEQ ID NO: 1
  Synthetic
  misc_feature (25)..(26): May be separated by a photocleavable moiety. In some instances, the photocleavable moitey is a 2-nitrobenzyl bond.

SEQ ID NO: 26 - RNA, 41 nt, Artificial Sequence; identical to SEQ ID NO: 1
  Synthetic
  misc_feature (26)..(27): May be separated by a photocleavable moiety. In some instances, the photocleavable moitey is a 2-nitrobenzyl bond.

SEQ ID NO: 27 - RNA, 41 nt, Artificial Sequence; identical to SEQ ID NO: 1
  Synthetic
  misc_feature (27)..(28): May be separated by a photocleavable moiety. In some instances, the photocleavable moitey is a 2-nitrobenzyl bond.

SEQ ID NO: 28 - RNA, 41 nt, Artificial Sequence; identical to SEQ ID NO: 1
  Synthetic
  misc_feature (28)..(29): May be separated by a photocleavable moiety. In some instances, the photocleavable moitey is a 2-nitrobenzyl bond.

SEQ ID NO: 29 - RNA, 41 nt, Artificial Sequence; identical to SEQ ID NO: 1
  Synthetic
  misc_feature (29)..(30): May be separated by a photocleavable moiety. In some instances, the photocleavable moitey is a 2-nitrobenzyl bond.

SEQ ID NO: 30 - RNA, 41 nt, Artificial Sequence; identical to SEQ ID NO: 1
  Synthetic
  misc_feature (30)..(31): May be separated by a photocleavable moiety. In some instances, the photocleavable moitey is a 2-nitrobenzyl bond.

SEQ ID NO: 31 - RNA, 41 nt, Artificial Sequence; identical to SEQ ID NO: 1
  Synthetic
  misc_feature (31)..(32): May be separated by a photocleavable moiety. In some instances, the photocleavable moitey is a 2-nitrobenzyl bond.

SEQ ID NO: 32 - RNA, 41 nt, Artificial Sequence; identical to SEQ ID NO: 1
  Synthetic
  misc_feature (32)..(33): May be separated by a photocleavable moiety. In some instances, the photocleavable moitey is a 2-nitrobenzyl bond.

SEQ ID NO: 33 - RNA, 41 nt, Artificial Sequence; identical to SEQ ID NO: 1
  Synthetic
  misc_feature (33)..(34): May be separated by a photocleavable moiety. In some instances, the photocleavable moitey is a 2-nitrobenzyl bond.

SEQ ID NO: 34 - RNA, 41 nt, Artificial Sequence; identical to SEQ ID NO: 1
  Synthetic
  misc_feature (34)..(35): May be separated by a photocleavable moiety. In some instances, the photocleavable moitey is a 2-nitrobenzyl bond.

SEQ ID NO: 35 - RNA, 41 nt, Artificial Sequence; identical to SEQ ID NO: 1
  Synthetic
  misc_feature (35)..(36): May be separated by a photocleavable moiety. In some instances, the photocleavable moitey is a 2-nitrobenzyl bond.

SEQ ID NO: 36 - RNA, 41 nt, Artificial Sequence; identical to SEQ ID NO: 1
  Synthetic
  misc_feature (36)..(37): May be separated by a photocleavable moiety. In some instances, the photocleavable moitey is a 2-nitrobenzyl bond.

SEQ ID NO: 37 - RNA, 41 nt, Artificial Sequence; identical to SEQ ID NO: 1
  Synthetic
  misc_feature (37)..(38): May be separated by a photocleavable moiety. In some instances, the photocleavable moitey is a 2-nitrobenzyl bond.

SEQ ID NO: 38 - RNA, 41 nt, Artificial Sequence; identical to SEQ ID NO: 1
  Synthetic
  misc_feature (38)..(39): May be separated by a photocleavable moiety. In some instances, the photocleavable moitey is a 2-nitrobenzyl bond.

SEQ ID NO: 39 - RNA, 41 nt, Artificial Sequence; identical to SEQ ID NO: 1
  Synthetic
  misc_feature (39)..(40): May be separated by a photocleavable moiety. In some instances, the photocleavable moitey is a 2-nitrobenzyl bond.

SEQ ID NO: 40 - RNA, 41 nt, Artificial Sequence; identical to SEQ ID NO: 1
  Synthetic
  misc_feature (40)..(41): May be separated by a photocleavable moiety. In some instances, the photocleavable moitey is a 2-nitrobenzyl bond.

SEQ ID NO: 41 - RNA, 106 nt, Artificial Sequence
  Synthetic
  misc_feature (2)..(2): n is a phosphorothioate
  misc_feature (4)..(4): n is a phosphorothioate
  misc_feature (6)..(6): n is a phosphorothioate
  misc_feature (8)..(9): May be separated by a photocleavable moiety. In some instances, the photocleavable moitey is a 2-nitrobenzyl bond.
  misc_feature (101)..(101): n is a phosphorothioate
  misc_feature (103)..(103): n is a phosphorothioate
  misc_feature (105)..(105): n is a phosphorothioate
cnanunagcugaugguaccccccguuuuagagcuagaaauagcaaguuaaaauaaggcuaguccguuaucaacuugaaaaaguggcaccgagucggugcunununu

SEQ ID NO: 42 - RNA, 106 nt, Artificial Sequence
  Synthetic
  misc_feature (2)..(2): n is a phosphorothioate
  misc_feature (4)..(4): n is a phosphorothioate
  misc_feature (6)..(6): n is a phosphorothioate
  misc_feature (8)..(9): May be separated by a photocleavable moiety. In some instances, the photocleavable moitey is a 2-nitrobenzyl bond.
  misc_feature (101)..(101): n is a phosphorothioate
  misc_feature (103)..(103): n is a phosphorothioate
  misc_feature (105)..(105): n is a phosphorothioate
gnancnccccuccaccccgccucguuuuagagcuagaaauagcaaguuaaaauaaggcuaguccguuaucaacuugaaaaaguggcaccgagucggugcunununu

SEQ ID NO: 43 - RNA, 106 nt, Artificial Sequence
  Synthetic
  misc_feature (2)..(2): n is a phosphorothioate
  misc_feature (4)..(4): n is a phosphorothioate
  misc_feature (6)..(6): n is a phosphorothioate
  misc_feature (8)..(9): May be separated by a photocleavable moiety. In some instances, the photocleavable moitey is a 2-nitrobenzyl bond.
  misc_feature (101)..(101): n is a phosphorothioate
  misc_feature (103)..(103): n is a phosphorothioate
  misc_feature (105)..(105): n is a phosphorothioate
gngnungggggcagcugacaaccguuuuagagcuagaaauagcaaguuaaaauaaggcuaguccguuaucaacuugaaaaaguggcaccgagucggugcunununu

SEQ ID NO: 44 - RNA, 20 nt, Artificial Sequence
  Synthetic
uaauuucuacucuuguagau

SEQ ID NO: 45 - RNA, 37 nt, Artificial Sequence
  Synthetic
  misc_feature (21)..(37): n is a, c, g, or u
uaauuucuacucuuguagaunnnnnnnnnnnnnnnnn

SEQ ID NO: 46 - RNA, 38 nt, Artificial Sequence
  Synthetic
  misc_feature (21)..(38): n is a, c, g, or u
uaauuucuacucuuguagaunnnnnnnnnnnnnnnnnn

SEQ ID NO: 47 - RNA, 39 nt, Artificial Sequence
  Synthetic
  misc_feature (21)..(39): n is a, c, g, or u
uaauuucuacucuuguagaunnnnnnnnnnnnnnnnnnn

SEQ ID NO: 48 - RNA, 40 nt, Artificial Sequence
  Synthetic
  misc_feature (21)..(40): n is a, c, g, or u
uaauuucuacucuuguagaunnnnnnnnnnnnnnnnnnnn

SEQ ID NO: 49 - RNA, 41 nt, Artificial Sequence
  Synthetic
  misc_feature (21)..(41): n is a, c, g, or u
uaauuucuacucuuguagaunnnnnnnnnnnnnnnnnnnnn

SEQ ID NO: 50 - RNA, 42 nt, Artificial Sequence
  Synthetic
  misc_feature (21)..(42): n is a, c, g, or u
uaauuucuacucuuguagaunnnnnnnnnnnnnnnnnnnnnn

SEQ ID NO: 51 - RNA, 43 nt, Artificial Sequence
  Synthetic
  misc_feature (21)..(43): n is a, c, g, or u
uaauuucuacucuuguagaunnnnnnnnnnnnnnnnnnnnnnn

SEQ ID NO: 52 - RNA, 44 nt, Artificial Sequence
  Synthetic
  misc_feature (21)..(44): n is a, c, g, or u
uaauuucuacucuuguagaunnnnnnnnnnnnnnnnnnnnnnnn

SEQ ID NO: 53 - RNA, 45 nt, Artificial Sequence
  Synthetic
  misc_feature (21)..(45): n is a, c, g, or u
uaauuucuacucuuguagaunnnnnnnnnnnnnnnnnnnnnnnnn

SEQ ID NO: 54 - skipped

SEQ ID NO: 55 - RNA, 21 nt, Artificial Sequence
  Synthetic
  misc_feature (21)..(21): n is a, c, g, or u
  repeat_unit (21)..(21): N may be repeated between 17 and 15 times
uaauuucuacucuuguagaun

SEQ ID NO: 56 - RNA, 41 nt, Artificial Sequence; identical to SEQ ID NO: 49
  Synthetic
  misc_feature (1)..(2): May be separated by a photocleavable moiety
  misc_feature (21)..(41): n is a, c, g, or u

SEQ ID NO: 57 - RNA, 41 nt, Artificial Sequence; identical to SEQ ID NO: 49
  Synthetic
  misc_feature (2)..(3): May be separated by a photocleavable moiety
  misc_feature (21)..(41): n is a, c, g, or u

SEQ ID NO: 58 - RNA, 41 nt, Artificial Sequence; identical to SEQ ID NO: 49
  Synthetic
  misc_feature (4)..(5): May be separated by a photocleavable moiety
  misc_feature (21)..(41): n is a, c, g, or u

SEQ ID NO: 59 - RNA, 41 nt, Artificial Sequence; identical to SEQ ID NO: 49
  Synthetic
  misc_feature (5)..(6): May be separated by a photocleavable moiety
  misc_feature (21)..(41): n is a, c, g, or u

SEQ ID NO: 60 - RNA, 41 nt, Artificial Sequence; identical to SEQ ID NO: 49
  Synthetic
  misc_feature (6)..(7): May be separated by a photocleavable moiety
  misc_feature (21)..(41): n is a, c, g, or u

SEQ ID NO: 61 - RNA, 41 nt, Artificial Sequence; identical to SEQ ID NO: 49
  Synthetic
  misc_feature (7)..(8): May be separated by a photocleavable moiety
  misc_feature (21)..(41): n is a, c, g, or u

SEQ ID NO: 62 - RNA, 41 nt, Artificial Sequence; identical to SEQ ID NO: 49
  Synthetic
  misc_feature (8)..(9): May be separated by a photocleavable moiety
  misc_feature (21)..(41): n is a, c, g, or u

SEQ ID NO: 63 - RNA, 41 nt, Artificial Sequence; identical to SEQ ID NO: 49
  Synthetic
  misc_feature (9)..(10): May be separated by a photocleavable moiety
  misc_feature (21)..(41): n is a, c, g, or u

SEQ ID NO: 64 - RNA, 41 nt, Artificial Sequence; identical to SEQ ID NO: 49
  Synthetic
  misc_feature (10)..(11): May be separated by a photocleavable moiety
  misc_feature (21)..(41): n is a, c, g, or u

SEQ ID NO: 65 - RNA, 41 nt, Artificial Sequence; identical to SEQ ID NO: 49
  Synthetic
  misc_feature (11)..(12): May be separated by a photocleavable moiety
  misc_feature (21)..(41): n is a, c, g, or u

SEQ ID NO: 66 - RNA, 41 nt, Artificial Sequence; identical to SEQ ID NO: 49
  Synthetic
  misc_feature (12)..(13): May be separated by a photocleavable moiety
  misc_feature (21)..(41): n is a, c, g, or u

SEQ ID NO: 67 - RNA, 41 nt, Artificial Sequence; identical to SEQ ID NO: 49
  Synthetic
  misc_feature (13)..(14): May be separated by a photocleavable moiety
  misc_feature (21)..(41): n is a, c, g, or u

SEQ ID NO: 68 - RNA, 41 nt, Artificial Sequence; identical to SEQ ID NO: 49
  Synthetic
  misc_feature (14)..(15): May be separated by a photocleavable moiety
  misc_feature (21)..(41): n is a, c, g, or u

SEQ ID NO: 69 - RNA, 41 nt, Artificial Sequence; identical to SEQ ID NO: 49
  Synthetic
  misc_feature (15)..(16): May be separated by a photocleavable moiety
  misc_feature (21)..(41): n is a, c, g, or u

SEQ ID NO: 70 - RNA, 41 nt, Artificial Sequence; identical to SEQ ID NO: 49
  Synthetic
  misc_feature (16)..(17): May be separated by a photocleavable moiety
  misc_feature (21)..(41): n is a, c, g, or u

SEQ ID NO: 71 - RNA, 41 nt, Artificial Sequence; identical to SEQ ID NO: 49
  Synthetic
  misc_feature (17)..(18): May be separated by a photocleavable moiety
  misc_feature (21)..(41): n is a, c, g, or u

SEQ ID NO: 72 - RNA, 41 nt, Artificial Sequence; identical to SEQ ID NO: 49
  Synthetic
  misc_feature (18)..(19): May be separated by a photocleavable moiety
  misc_feature (21)..(41): n is a, c, g, or u

SEQ ID NO: 73 - RNA, 41 nt, Artificial Sequence; identical to SEQ ID NO: 49
  Synthetic
  misc_feature (19)..(20): May be separated by a photocleavable moiety
  misc_feature (21)..(41): n is a, c, g, or u

SEQ ID NO: 74 - RNA, 41 nt, Artificial Sequence; identical to SEQ ID NO: 49
  Synthetic
  misc_feature (20)..(21): May be separated by a photocleavable moiety
  misc_feature (21)..(41): n is a, c, g, or u

SEQ ID NO: 75 - RNA, 41 nt, Artificial Sequence; identical to SEQ ID NO: 49
  Synthetic
  misc_feature (21)..(22): May be separated by a photocleavable moiety
  misc_feature (21)..(41): n is a, c, g, or u

SEQ ID NO: 76 - RNA, 41 nt, Artificial Sequence; identical to SEQ ID NO: 49
  Synthetic
  misc_feature (21)..(41): n is a, c, g, or u
  misc_feature (22)..(23): May be separated by a photocleavable moiety

SEQ ID NO: 77 - RNA, 41 nt, Artificial Sequence; identical to SEQ ID NO: 49
  Synthetic
  misc_feature (21)..(41): n is a, c, g, or u
  misc_feature (23)..(24): May be separated by a photocleavable moiety

SEQ ID NO: 78 - RNA, 41 nt, Artificial Sequence; identical to SEQ ID NO: 49
  Synthetic
  misc_feature (21)..(41): n is a, c, g, or u
  misc_feature (24)..(25): May be separated by a photocleavable moiety

SEQ ID NO: 79 - RNA, 41 nt, Artificial Sequence; identical to SEQ ID NO: 49
  Synthetic
  misc_feature (21)..(41): n is a, c, g, or u
  misc_feature (25)..(26): May be separated by a photocleavable moiety

SEQ ID NO: 80 - RNA, 41 nt, Artificial Sequence; identical to SEQ ID NO: 49
  Synthetic
  misc_feature (21)..(41): n is a, c, g, or u
  misc_feature (26)..(27): May be separated by a photocleavable moiety

SEQ ID NO: 81 - RNA, 41 nt, Artificial Sequence; identical to SEQ ID NO: 49
  Synthetic
  misc_feature (21)..(41): n is a, c, g, or u
  misc_feature (27)..(28): May be separated by a photocleavable moiety

SEQ ID NO: 82 - RNA, 41 nt, Artificial Sequence; identical to SEQ ID NO: 49
  Synthetic
  misc_feature (21)..(41): n is a, c, g, or u
  misc_feature (28)..(29): May be separated by a photocleavable moiety

SEQ ID NO: 83 - RNA, 41 nt, Artificial Sequence; identical to SEQ ID NO: 49
  Synthetic
  misc_feature (21)..(41): n is a, c, g, or u
  misc_feature (29)..(30): May be separated by a photocleavable moiety

SEQ ID NO: 84 - RNA, 41 nt, Artificial Sequence; identical to SEQ ID NO: 49
  Synthetic
  misc_feature (21)..(41): n is a, c, g, or u
  misc_feature (30)..(31): May be separated by a photocleavable moiety

SEQ ID NO: 85 - RNA, 41 nt, Artificial Sequence; identical to SEQ ID NO: 49
  Synthetic
  misc_feature (21)..(41): n is a, c, g, or u
  misc_feature (31)..(32): May be separated by a photocleavable moiety

SEQ ID NO: 86 - RNA, 41 nt, Artificial Sequence; identical to SEQ ID NO: 49
  Synthetic
  misc_feature (21)..(41): n is a, c, g, or u
  misc_feature (32)..(33): May be separated by a photocleavable moiety

SEQ ID NO: 87 - RNA, 41 nt, Artificial Sequence; identical to SEQ ID NO: 49
  Synthetic
  misc_feature (21)..(41): n is a, c, g, or u
  misc_feature (33)..(34): May be separated by a photocleavable moiety

SEQ ID NO: 88 - RNA, 41 nt, Artificial Sequence; identical to SEQ ID NO: 49
  Synthetic
  misc_feature (21)..(41): n is a, c, g, or u
  misc_feature (34)..(35): May be separated by a photocleavable moiety

SEQ ID NO: 89 - RNA, 41 nt, Artificial Sequence; identical to SEQ ID NO: 49
  Synthetic
  misc_feature (21)..(41): n is a, c, g, or u
  misc_feature (35)..(36): May be separated by a photocleavable moiety

SEQ ID NO: 90 - RNA, 41 nt, Artificial Sequence; identical to SEQ ID NO: 49
  Synthetic
  misc_feature (21)..(41): n is a, c, g, or u
  misc_feature (36)..(37): May be separated by a photocleavable moiety

SEQ ID NO: 91 - RNA, 41 nt, Artificial Sequence; identical to SEQ ID NO: 49
  Synthetic
  misc_feature (21)..(41): n is a, c, g, or u
  misc_feature (37)..(38): May be separated by a photocleavable moiety

SEQ ID NO: 92 - RNA, 41 nt, Artificial Sequence; identical to SEQ ID NO: 49
  Synthetic
  misc_feature (21)..(41): n is a, c, g, or u
  misc_feature (38)..(39): May be separated by a photocleavable moiety

SEQ ID NO: 93 - RNA, 41 nt, Artificial Sequence; identical to SEQ ID NO: 49
  Synthetic
  misc_feature (21)..(41): n is a, c, g, or u
  misc_feature (39)..(40): May be separated by a photocleavable moiety

SEQ ID NO: 94 - RNA, 41 nt, Artificial Sequence; identical to SEQ ID NO: 49
  Synthetic
  misc_feature (21)..(39): n is a, c, g, or u
  misc_feature (40)..(41): May be separated by a photocleavable moiety

SEQ ID NO: 95 - RNA, 40 nt, Artificial Sequence
  Synthetic
  misc_feature (1)..(3): May be separated by a photocleavable moiety.
  misc_feature (2)..(2): May be absent.
  misc_feature (20)..(40): n is a, c, g, or u
uauuucuacucuuguagaunnnnnnnnnnnnnnnnnnnnn

SEQ ID NO: 96 - RNA, 40 nt, Artificial Sequence; identical to SEQ ID NO: 95
  Synthetic
  misc_feature (2)..(4): May be separated by a photocleavable moiety.
  misc_feature (3)..(3): May be absent.
  misc_feature (20)..(40): n is a, c, g, or u

SEQ ID NO: 97 - RNA, 40 nt, Artificial Sequence; identical to SEQ ID NO: 95
  Synthetic
  misc_feature (3)..(5): May be separated by a photocleavable moiety.
  misc_feature (4)..(4): May be absent.
  misc_feature (20)..(40): n is a, c, g, or u

SEQ ID NO: 98 - RNA, 40 nt, Artificial Sequence; identical to SEQ ID NO: 95
  Synthetic
  misc_feature (4)..(6): May be separated by a photocleavable moiety.
  misc_feature (5)..(5): May be absent.
  misc_feature (20)..(40): n is a, c, g, or u

SEQ ID NO: 99 - RNA, 40 nt, Artificial Sequence; identical to SEQ ID NO: 95
  Synthetic
  misc_feature (5)..(7): May be separated by a photocleavable moiety.
  misc_feature (6)..(6): May be absent.
  misc_feature (20)..(40): n is a, c, g, or u

SEQ ID NO: 100 - RNA, 40 nt, Artificial Sequence; identical to SEQ ID NO: 95
  Synthetic
  misc_feature (6)..(8): May be separated by a photocleavable moiety.
  misc_feature (7)..(7): May be absent.
  misc_feature (20)..(40): n is a, c, g, or u

SEQ ID NO: 101 - RNA, 40 nt, Artificial Sequence; identical to SEQ ID NO: 95
  Synthetic
  misc_feature (7)..(9): May be separated by a photocleavable moiety.
  misc_feature (8)..(8): May be absent.
  misc_feature (20)..(40): n is a, c, g, or u

SEQ ID NO: 102 - RNA, 40 nt, Artificial Sequence; identical to SEQ ID NO: 95
  Synthetic
  misc_feature (8)..(10): May be separated by a photocleavable moiety.
  misc_feature (9)..(9): May be absent.
  misc_feature (20)..(40): n is a, c, g, or u

SEQ ID NO: 103 - RNA, 40 nt, Artificial Sequence; identical to SEQ ID NO: 95
  Synthetic
  misc_feature (9)..(11): May be separated by a photocleavable moiety.
  misc_feature (10)..(10): May be absent.
  misc_feature (20)..(40): n is a, c, g, or u

SEQ ID NO: 104 - RNA, 40 nt, Artificial Sequence; identical to SEQ ID NO: 95
  Synthetic
  misc_feature (10)..(12): May be separated by a photocleavable moiety.
  misc_feature (11)..(11): May be absent.
  misc_feature (20)..(40): n is a, c, g, or u

SEQ ID NO: 105 - RNA, 40 nt, Artificial Sequence; identical to SEQ ID NO: 95
  Synthetic
  misc_feature (11)..(13): May be separated by a photocleavable moiety.
  misc_feature (12)..(12): May be absent.
  misc_feature (20)..(40): n is a, c, g, or u

SEQ ID NO: 106 - RNA, 40 nt, Artificial Sequence; identical to SEQ ID NO: 95
  Synthetic
  misc_feature (12)..(14): May be separated by a photocleavable moiety.
  misc_feature (13)..(13): May be absent.
  misc_feature (20)..(40): n is a, c, g, or u

SEQ ID NO: 107 - RNA, 40 nt, Artificial Sequence; identical to SEQ ID NO: 95
  Synthetic
  misc_feature (13)..(15): May be separated by a photocleavable moiety.
  misc_feature (14)..(14): May be absent.
  misc_feature (20)..(40): n is a, c, g, or u

SEQ ID NO: 108 - RNA, 40 nt, Artificial Sequence; identical to SEQ ID NO: 95
  Synthetic
  misc_feature (14)..(16): May be separated by a photocleavable moiety.
  misc_feature (15)..(15): May be absent.
  misc_feature (20)..(40): n is a, c, g, or u

SEQ ID NO: 109 - RNA, 40 nt, Artificial Sequence; identical to SEQ ID NO: 95
  Synthetic
  misc_feature (15)..(17): May be separated by a photocleavable moiety.
  misc_feature (16)..(16): May be absent.
  misc_feature (20)..(40): n is a, c, g, or u

SEQ ID NO: 110 - RNA, 40 nt, Artificial Sequence; identical to SEQ ID NO: 95
  Synthetic
  misc_feature (16)..(18): May be separated by a photocleavable moiety.
  misc_feature (17)..(17): May be absent.
  misc_feature (20)..(40): n is a, c, g, or u

SEQ ID NO: 111 - RNA, 40 nt, Artificial Sequence; identical to SEQ ID NO: 95
  Synthetic
  misc_feature (17)..(19): May be separated by a photocleavable moiety.
  misc_feature (18)..(18): May be absent.
  misc_feature (20)..(40): n is a, c, g, or u

SEQ ID NO: 112 - RNA, 40 nt, Artificial Sequence; identical to SEQ ID NO: 95
  Synthetic
  misc_feature (18)..(20): May be separated by a photocleavable moiety.
  misc_feature (19)..(19): May be absent.
  misc_feature (20)..(40): n is a, c, g, or u

SEQ ID NO: 113 - RNA, 40 nt, Artificial Sequence; identical to SEQ ID NO: 95
  Synthetic
  misc_feature (19)..(21): May be separated by a photocleavable moiety.
  misc_feature (20)..(20): May be absent.
  misc_feature (20)..(40): n is a, c, g, or u

SEQ ID NO: 114 - RNA, 40 nt, Artificial Sequence; identical to SEQ ID NO: 95
  Synthetic
  misc_feature (20)..(22): May be separated by a photocleavable moiety.
  misc_feature (20)..(40): n is a, c, g, or u
  misc_feature (21)..(21): May be absent.

SEQ ID NO: 115 - RNA, 40 nt, Artificial Sequence; identical to SEQ ID NO: 95
  Synthetic
  misc_feature (20)..(40): n is a, c, g, or u
  misc_feature (21)..(23): May be separated by a photocleavable moiety.
  misc_feature (22)..(22): May be absent.

SEQ ID NO: 116 - RNA, 40 nt, Artificial Sequence; identical to SEQ ID NO: 95
  Synthetic
  misc_feature (20)..(40): n is a, c, g, or u
  misc_feature (22)..(24): May be separated by a photocleavable moiety.
  misc_feature (23)..(23): May be absent.

SEQ ID NO: 117 - RNA, 40 nt, Artificial Sequence; identical to SEQ ID NO: 95
  Synthetic
  misc_feature (20)..(40): n is a, c, g, or u
  misc_feature (23)..(25): May be separated by a photocleavable moiety.
  misc_feature (24)..(24): May be absent.

SEQ ID NO: 118 - RNA, 40 nt, Artificial Sequence; identical to SEQ ID NO: 95
  Synthetic
  misc_feature (20)..(40): n is a, c, g, or u
  misc_feature (24)..(26): May be separated by a photocleavable moiety.
  misc_feature (25)..(25): May be absent.

SEQ ID NO: 119 - RNA, 40 nt, Artificial Sequence; identical to SEQ ID NO: 95
  Synthetic
  misc_feature (20)..(40): n is a, c, g, or u
  misc_feature (25)..(27): May be separated by a photocleavable moiety.
  misc_feature (26)..(26): May be absent.

SEQ ID NO: 120 - RNA, 40 nt, Artificial Sequence; identical to SEQ ID NO: 95
  Synthetic
  misc_feature (20)..(40): n is a, c, g, or u
  misc_feature (26)..(28): May be separated by a photocleavable moiety.
  misc_feature (27)..(27): May be absent.

SEQ ID NO: 121 - RNA, 40 nt, Artificial Sequence; identical to SEQ ID NO: 95
  Synthetic
  misc_feature (20)..(40): n is a, c, g, or u
  misc_feature (27)..(29): May be separated by a photocleavable moiety.
  misc_feature (28)..(28): May be absent.

SEQ ID NO: 122 - RNA, 40 nt, Artificial Sequence; identical to SEQ ID NO: 95
  Synthetic
  misc_feature (20)..(40): n is a, c, g, or u
  misc_feature (28)..(30): May be separated by a photocleavable moiety.
  misc_feature (29)..(29): May be absent.

SEQ ID NO: 123 - RNA, 40 nt, Artificial Sequence; identical to SEQ ID NO: 95
  Synthetic
  misc_feature (20)..(40): n is a, c, g, or u
  misc_feature (29)..(31): May be separated by a photocleavable moiety.
  misc_feature (30)..(30): May be absent.

SEQ ID NO: 124 - RNA, 40 nt, Artificial Sequence; identical to SEQ ID NO: 95
  Synthetic
  misc_feature (20)..(40): n is a, c, g, or u
  misc_feature (30)..(32): May be separated by a photocleavable moiety.
  misc_feature (31)..(31): May be absent.

SEQ ID NO: 125 - RNA, 40 nt, Artificial Sequence; identical to SEQ ID NO: 95
  Synthetic
  misc_feature (20)..(40): n is a, c, g, or u
  misc_feature (31)..(33): May be separated by a photocleavable moiety.
  misc_feature (32)..(32): May be absent.

SEQ ID NO: 126 - RNA, 40 nt, Artificial Sequence; identical to SEQ ID NO: 95
  Synthetic
  misc_feature (20)..(40): n is a, c, g, or u
  misc_feature (32)..(34): May be separated by a photocleavable moiety.
  misc_feature (33)..(33): May be absent.

SEQ ID NO: 127 - RNA, 40 nt, Artificial Sequence; identical to SEQ ID NO: 95
  Synthetic
  misc_feature (20)..(40): n is a, c, g, or u
  misc_feature (33)..(35): May be separated by a photocleavable moiety.
  misc_feature (34)..(34): May be absent.

SEQ ID NO: 128 - RNA, 40 nt, Artificial Sequence; identical to SEQ ID NO: 95
  Synthetic
  misc_feature (20)..(40): n is a, c, g, or u
  misc_feature (34)..(36): May be separated by a photocleavable moiety.
  misc_feature (35)..(35): May be absent.

SEQ ID NO: 129 - RNA, 40 nt, Artificial Sequence; identical to SEQ ID NO: 95
  Synthetic
  misc_feature (20)..(40): n is a, c, g, or u
  misc_feature (35)..(37): May be separated by a photocleavable moiety.
  misc_feature (36)..(36): May be absent.

SEQ ID NO: 130 - RNA, 40 nt, Artificial Sequence; identical to SEQ ID NO: 95
  Synthetic
  misc_feature (20)..(40): n is a, c, g, or u
  misc_feature (36)..(38): May be separated by a photocleavable moiety.
  misc_feature (37)..(37): May be absent.

SEQ ID NO: 131 - RNA, 40 nt, Artificial Sequence; identical to SEQ ID NO: 95
  Synthetic
  misc_feature (20)..(40): n is a, c, g, or u
  misc_feature (37)..(39): May be separated by a photocleavable moiety.
  misc_feature (38)..(38): May be absent.

SEQ ID NO: 132 - RNA, 40 nt, Artificial Sequence; identical to SEQ ID NO: 95
  Synthetic
  misc_feature (20)..(40): n is a, c, g, or u
  misc_feature (38)..(40): May be separated by a photocleavable moiety.
  misc_feature (39)..(39): May be absent.

SEQ ID NO: 133 - RNA, 40 nt, Artificial Sequence; identical to SEQ ID NO: 95
  Synthetic
  misc_feature (20)..(40): n is a, c, g, or u
  misc_feature (39)..(40): May be separated by a photocleavable moiety.
  misc_feature (40)..(40): May be absent.

SEQ ID NO: 134 - RNA, 40 nt, Artificial Sequence
  Synthetic
  misc_feature (1)..(3): May be separated by a photocleavable moiety. In some instances, the photocleavable moitey is a 2-nitrobenzyl bond.
  misc_feature (2)..(2): May be absent.
uauuucuacucuuguagauagcggggacucccgagaccag

SEQ ID NO: 135 - RNA, 40 nt, Artificial Sequence; identical to SEQ ID NO: 134
  Synthetic
  misc_feature (2)..(4): May be separated by a photocleavable moiety. In some instances, the photocleavable moitey is a 2-nitrobenzyl bond.
  misc_feature (3)..(3): May be absent.

SEQ ID NO: 136 - RNA, 40 nt, Artificial Sequence; identical to SEQ ID NO: 134
  Synthetic
  misc_feature (3)..(5): May be separated by a photocleavable moiety. In some instances, the photocleavable moitey is a 2-nitrobenzyl bond.
  misc_feature (4)..(4): May be absent.

SEQ ID NO: 137 - RNA, 40 nt, Artificial Sequence; identical to SEQ ID NO: 134
  Synthetic
  misc_feature (4)..(6): May be separated by a photocleavable moiety. In some instances, the photocleavable moitey is a 2-nitrobenzyl bond.
  misc_feature (5)..(5): May be absent.

SEQ ID NO: 138 - RNA, 40 nt, Artificial Sequence; identical to SEQ ID NO: 134
  Synthetic
  misc_feature (5)..(7): May be separated by a photocleavable moiety. In some instances, the photocleavable moitey is a 2-nitrobenzyl bond.
  misc_feature (6)..(6): May be absent.

SEQ ID NO: 139 - RNA, 40 nt, Artificial Sequence; identical to SEQ ID NO: 134
  Synthetic
  misc_feature (6)..(8): May be separated by a photocleavable moiety. In some instances, the photocleavable moitey is a 2-nitrobenzyl bond.
  misc_feature (7)..(7): May be absent.

SEQ ID NO: 140 - RNA, 40 nt, Artificial Sequence; identical to SEQ ID NO: 134
  Synthetic
  misc_feature (7)..(9): May be separated by a photocleavable moiety. In some instances, the photocleavable moitey is a 2-nitrobenzyl bond.
  misc_feature (8)..(8): May be absent.

SEQ ID NO: 141 - RNA, 40 nt, Artificial Sequence; identical to SEQ ID NO: 134
  Synthetic
  misc_feature (8)..(10): May be separated by a photocleavable moiety. In some instances, the photocleavable moitey is a 2-nitrobenzyl bond.
  misc_feature (9)..(9): May be absent.

SEQ ID NO: 142 - RNA, 40 nt, Artificial Sequence; identical to SEQ ID NO: 134
  Synthetic
  misc_feature (9)..(11): May be separated by a photocleavable moiety. In some instances, the photocleavable moitey is a 2-nitrobenzyl bond.
  misc_feature (10)..(10): May be absent.

SEQ ID NO: 143 - RNA, 40 nt, Artificial Sequence; identical to SEQ ID NO: 134
  Synthetic
  misc_feature (10)..(12): May be separated by a photocleavable moiety. In some instances, the photocleavable moitey is a 2-nitrobenzyl bond.
  misc_feature (11)..(11): May be absent.

SEQ ID NO: 144 - RNA, 40 nt, Artificial Sequence; identical to SEQ ID NO: 134
  Synthetic
  misc_feature (11)..(13): May be separated by a photocleavable moiety. In some instances, the photocleavable moitey is a 2-nitrobenzyl bond.
  misc_feature (12)..(12): May be absent.

SEQ ID NO: 145 - RNA, 40 nt, Artificial Sequence; identical to SEQ ID NO: 134
  Synthetic
  misc_feature (12)..(14): May be separated by a photocleavable moiety. In some instances, the photocleavable moitey is a 2-nitrobenzyl bond.
  misc_feature (13)..(13): May be absent.

SEQ ID NO: 146 - RNA, 40 nt, Artificial Sequence; identical to SEQ ID NO: 134
  Synthetic
  misc_feature (13)..(15): May be separated by a photocleavable moiety. In some instances, the photocleavable moitey is a 2-nitrobenzyl bond.
  misc_feature (14)..(14): May be absent.

SEQ ID NO: 147 - RNA, 40 nt, Artificial Sequence; identical to SEQ ID NO: 134
  Synthetic
  misc_feature (14)..(16): May be separated by a photocleavable moiety. In some instances, the photocleavable moitey is a 2-nitrobenzyl bond.
  misc_feature (15)..(15): May be absent.

SEQ ID NO: 148 - RNA, 40 nt, Artificial Sequence; identical to SEQ ID NO: 134
  Synthetic
  misc_feature (15)..(17): May be separated by a photocleavable moiety. In some instances, the photocleavable moitey is a 2-nitrobenzyl bond.
  misc_feature (16)..(16): May be absent.

SEQ ID NO: 149 - RNA, 40 nt, Artificial Sequence; identical to SEQ ID NO: 134
  Synthetic
  misc_feature (16)..(18): May be separated by a photocleavable moiety. In some instances, the photocleavable moitey is a 2-nitrobenzyl bond.
  misc_feature (17)..(17): May be absent.

SEQ ID NO: 150 - RNA, 40 nt, Artificial Sequence; identical to SEQ ID NO: 134
  Synthetic
  misc_feature (17)..(19): May be separated by a photocleavable moiety. In some instances, the photocleavable moitey is a 2-nitrobenzyl bond.
  misc_feature (18)..(18): May be absent.

SEQ ID NO: 151 - RNA, 40 nt, Artificial Sequence; identical to SEQ ID NO: 134
  Synthetic
  misc_feature (18)..(20): May be separated by a photocleavable moiety. In some instances, the photocleavable moitey is a 2-nitrobenzyl bond.
  misc_feature (19)..(19): May be absent.

SEQ ID NO: 152 - RNA, 40 nt, Artificial Sequence; identical to SEQ ID NO: 134
  Synthetic
  misc_feature (19)..(21): May be separated by a photocleavable moiety. In some instances, the photocleavable moitey is a 2-nitrobenzyl bond.
  misc_feature (20)..(20): May be absent.

SEQ ID NO: 153 - RNA, 40 nt, Artificial Sequence; identical to SEQ ID NO: 134
  Synthetic
  misc_feature (20)..(22): May be separated by a photocleavable moiety. In some instances, the photocleavable moitey is a 2-nitrobenzyl bond.
  misc_feature (21)..(21): May be absent.

SEQ ID NO: 154 - RNA, 40 nt, Artificial Sequence; identical to SEQ ID NO: 134
  Synthetic
  misc_feature (21)..(23): May be separated by a photocleavable moiety. In some instances, the photocleavable moitey is a 2-nitrobenzyl bond.
  misc_feature (22)..(22): May be absent.

SEQ ID NO: 155 - RNA, 40 nt, Artificial Sequence; identical to SEQ ID NO: 134
  Synthetic
  misc_feature (22)..(24): May be separated by a photocleavable moiety. In some instances, the photocleavable moitey is a 2-nitrobenzyl bond.
  misc_feature (23)..(23): May be absent.

SEQ ID NO: 156 - RNA, 40 nt, Artificial Sequence; identical to SEQ ID NO: 134
  Synthetic
  misc_feature (23)..(25): May be separated by a photocleavable moiety. In some instances, the photocleavable moitey is a 2-nitrobenzyl bond.
  misc_feature (24)..(24): May be absent.

SEQ ID NO: 157 - RNA, 40 nt, Artificial Sequence; identical to SEQ ID NO: 134
  Synthetic
  misc_feature (24)..(26): May be separated by a photocleavable moiety. In some instances, the photocleavable moitey is a 2-nitrobenzyl bond.
  misc_feature (25)..(25): May be absent.

SEQ ID NO: 158 - RNA, 40 nt, Artificial Sequence; identical to SEQ ID NO: 134
  Synthetic
  misc_feature (25)..(27): May be separated by a photocleavable moiety. In some instances, the photocleavable moitey is a 2-nitrobenzyl bond.
  misc_feature (26)..(26): May be absent.

SEQ ID NO: 159 - RNA, 40 nt, Artificial Sequence; identical to SEQ ID NO: 134
  Synthetic
  misc_feature (26)..(28): May be separated by a photocleavable moiety. In some instances, the photocleavable moitey is a 2-nitrobenzyl bond.
  misc_feature (27)..(27): May be absent.

SEQ ID NO: 160 - RNA, 40 nt, Artificial Sequence; identical to SEQ ID NO: 134
  Synthetic
  misc_feature (27)..(29): May be separated by a photocleavable moiety. In some instances, the photocleavable moitey is a 2-nitrobenzyl bond.
  misc_feature (28)..(28): May be absent.

SEQ ID NO: 161 - RNA, 40 nt, Artificial Sequence; identical to SEQ ID NO: 134
  Synthetic
  misc_feature (28)..(30): May be separated by a photocleavable moiety. In some instances, the photocleavable moitey is a 2-nitrobenzyl bond.
  misc_feature (29)..(29): May be absent.

SEQ ID NO: 162 - RNA, 40 nt, Artificial Sequence; identical to SEQ ID NO: 134
  Synthetic
  misc_feature (29)..(31): May be separated by a photocleavable moiety. In some instances, the photocleavable moitey is a 2-nitrobenzyl bond.
  misc_feature (30)..(30): May be absent.

SEQ ID NO: 163 - RNA, 40 nt, Artificial Sequence; identical to SEQ ID NO: 134
  Synthetic
  misc_feature (30)..(32): May be separated by a photocleavable moiety. In some instances, the photocleavable moitey is a 2-nitrobenzyl bond.
  misc_feature (31)..(31): May be absent.

SEQ ID NO: 164 - RNA, 40 nt, Artificial Sequence; identical to SEQ ID NO: 134
  Synthetic
  misc_feature (31)..(33): May be separated by a photocleavable moiety. In some instances, the photocleavable moitey is a 2-nitrobenzyl bond.
  misc_feature (32)..(32): May be absent.

SEQ ID NO: 165 - RNA, 40 nt, Artificial Sequence; identical to SEQ ID NO: 134
  Synthetic
  misc_feature (32)..(34): May be separated by a photocleavable moiety. In some instances, the photocleavable moitey is a 2-nitrobenzyl bond.
  misc_feature (33)..(33): May be absent.

SEQ ID NO: 166 - RNA, 40 nt, Artificial Sequence; identical to SEQ ID NO: 134
  Synthetic
  misc_feature (33)..(35): May be separated by a photocleavable moiety. In some instances, the photocleavable moitey is a 2-nitrobenzyl bond.
  misc_feature (34)..(34): May be absent.

SEQ ID NO: 167 - RNA, 40 nt, Artificial Sequence; identical to SEQ ID NO: 134
  Synthetic
  misc_feature (34)..(36): May be separated by a photocleavable moiety. In some instances, the photocleavable moitey is a 2-nitrobenzyl bond.
  misc_feature (35)..(35): May be absent.

SEQ ID NO: 168 - RNA, 40 nt, Artificial Sequence; identical to SEQ ID NO: 134
  Synthetic
  misc_feature (35)..(37): May be separated by a photocleavable moiety. In some instances, the photocleavable moitey is a 2-nitrobenzyl bond.
  misc_feature (36)..(36): May be absent.

SEQ ID NO: 169 - RNA, 40 nt, Artificial Sequence; identical to SEQ ID NO: 134
  Synthetic
  misc_feature (36)..(38): May be separated by a photocleavable moiety. In some instances, the photocleavable moitey is a 2-nitrobenzyl bond.
  misc_feature (37)..(37): May be absent.

SEQ ID NO: 170 - RNA, 40 nt, Artificial Sequence; identical to SEQ ID NO: 134
  Synthetic
  misc_feature (37)..(39): May be separated by a photocleavable moiety. In some instances, the photocleavable moitey is a 2-nitrobenzyl bond.
  misc_feature (38)..(38): May be absent.

SEQ ID NO: 171 - RNA, 40 nt, Artificial Sequence; identical to SEQ ID NO: 134
  Synthetic
  misc_feature (38)..(40): May be separated by a photocleavable moiety. In some instances, the photocleavable moitey is a 2-nitrobenzyl bond.
  misc_feature (39)..(39): May be absent.

SEQ ID NO: 172 - RNA, 40 nt, Artificial Sequence; identical to SEQ ID NO: 134
  Synthetic
  misc_feature (39)..(40): May be separated by a photocleavable moiety. In some instances, the photocleavable moitey is a 2-nitrobenzyl bond.
  misc_feature (40)..(40): May be absent.

SEQ ID NO: 173 - RNA, 20 nt, Artificial Sequence
  Synthetic
cauagcugaugguacccccc

SEQ ID NO: 174 - RNA, 20 nt, Artificial Sequence
  Synthetic
  misc_feature (5)..(7): May be separated by a photocleavable moiety. In some instances, the photocleavable moitey is a 2-nitrobenzyl bond.
  misc_feature (6)..(6): May be absent.
cauagcugaugguacccccc

SEQ ID NO: 175 - RNA, 20 nt, Artificial Sequence
  Synthetic
gacccccuccaccccgccuc

SEQ ID NO: 176 - RNA, 20 nt, Artificial Sequence
  Synthetic
  misc_feature (5)..(7): May be separated by a photocleavable moiety. In some instances, the photocleavable moitey is a 2-nitrobenzyl bond.
  misc_feature (6)..(6): May be absent.
gacccccuccaccccgccuc

SEQ ID NO: 177 - RNA, 20 nt, Artificial Sequence
  Synthetic
ggugggggcagcugacaacc

SEQ ID NO: 178 - RNA, 20 nt, Artificial Sequence
  Synthetic
  misc_feature (5)..(7): May be separated by a photocleavable moiety. In some instances, the photocleavable moitey is a 2-nitrobenzyl bond.
  misc_feature (6)..(6): May be absent.
ggugggggcagcugacaacc

SEQ ID NO: 179 - RNA, 20 nt, Artificial Sequence
  Synthetic
uuuaucacaggcuccaggaa

SEQ ID NO: 180 - RNA, 20 nt, Artificial Sequence
  Synthetic
  misc_feature (5)..(7): May be separated by a photocleavable moiety. In some instances, the photocleavable moitey is a 2-nitrobenzyl bond.
  misc_feature (6)..(6): May be absent.
uuuaucacaggcuccaggaa

SEQ ID NO: 181 - RNA, 99 nt, Artificial Sequence
  Synthetic
  misc_feature (5)..(7): May be separated by a photocleavable moiety.
  misc_feature (6)..(6): May be absent.
cauagugaugguaccccccguuuuagagcuagaaauagcaaguuaaaauaaggcuaguccguuaucaacuugaaaaaguggcaccgagucggugcuuuu

SEQ ID NO: 182 - RNA, 99 nt, Artificial Sequence
  Synthetic
  misc_feature (5)..(7): May be separated by a photocleavable moiety.
  misc_feature (6)..(6): May be absent.
gaccccuccaccccgccucguuuuagagcuagaaauagcaaguuaaaauaaggcuaguccguuaucaacuugaaaaaguggcaccgagucggugcuuuu

SEQ ID NO: 183 - RNA, 99 nt, Artificial Sequence
  Synthetic
  misc_feature (5)..(7): May be separated by a photocleavable moiety.
  misc_feature (6)..(6): May be absent.
gguggggcagcugacaaccguuuuagagcuagaaauagcaaguuaaaauaaggcuaguccguuaucaacuugaaaaaguggcaccgagucggugcuuuu

SEQ ID NO: 184 - RNA, 99 nt, Artificial Sequence
  Synthetic
  misc_feature (5)..(7): May be separated by a photocleavable moiety.
  misc_feature (6)..(6): May be absent.
uuuauacaggcuccaggaaguuuuagagcuagaaauagcaaguuaaaauaaggcuaguccguuaucaacuugaaaaaguggcaccgagucggugcuuuu

SEQ ID NO: 185 - RNA, 40 nt, Artificial Sequence
  Synthetic
  misc_feature (11)..(13): May be separated by a photocleavable moiety.
  misc_feature (12)..(12): May be absent.
uaauuucuacuuuguagauagcggggacucccgagaccag

SEQ ID NO: 186 - RNA, 40 nt, Artificial Sequence
  Synthetic
  misc_feature (12)..(14): May be separated by a photocleavable moiety.
  misc_feature (13)..(13): May be absent.
uaauuucuacucuguagauagcggggacucccgagaccag

SEQ ID NO: 187 - RNA, 40 nt, Artificial Sequence; identical to SEQ ID NO: 186
  Synthetic
  misc_feature (13)..(15): May be separated by a photocleavable moiety.
  misc_feature (14)..(14): May be absent.

SEQ ID NO: 188 - RNA, 40 nt, Artificial Sequence
  Synthetic
  misc_feature (33)..(35): May be separated by a photocleavable moiety.
  misc_feature (34)..(34): May be absent.
uaauuucuacucuuguagauagcggggacucccagaccag

SEQ ID NO: 189 - RNA, 40 nt, Artificial Sequence
  Synthetic
  misc_feature (35)..(37): May be separated by a photocleavable moiety.
  misc_feature (36)..(36): May be absent.
uaauuucuacucuuguagauagcggggacucccgaaccag

SEQ ID NO: 190 - RNA, 40 nt, Artificial Sequence
  Synthetic
  misc_feature (36)..(38): May be separated by a photocleavable moiety.
  misc_feature (37)..(37): May be absent.
uaauuucuacucuuguagauagcggggacucccgagccag

SEQ ID NO: 191 - RNA, 80 nt, Artificial Sequence
  Synthetic
guuuuagagcuagaaauagcaaguuaaaauaaggcuaguccguuaucaacuugaaaaaguggcaccgagucggugcuuuu

SEQ ID NO: 192 - protein, 4 aa, Artificial Sequence
  Synthetic
NTTN

SEQ ID NO: 193 - RNA, 41 nt, Artificial Sequence; identical to SEQ ID NO: 49
  Synthetic
  misc_feature (3)..(4): May be separated by a photocleavable moiety
  misc_feature (21)..(41): n is a, c, g, or u